SEQUENCE LISTING (56 sequences)

SEQ ID NO: 1 - protein, 1270 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHSYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 2 - protein, 1328 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDQPDLAPGLTTIGASPTQTVTLVTQPVVTKETAISKLEMPSSLMLEVPTHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 3 - protein, 1340 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDTM

SEQ ID NO: 4 - protein, 1197 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 5 - protein, 98 aa, synthetic construct
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 6 - protein, 95 aa, synthetic construct
MSGTSGAFEFKLPDIGEGIHEGEIVKWFVKPGDEVNEDDVLCEVQNDKAVVEIPSPVKGKVLEILVPEGTVATVGQTLITLDAPGYENMTTGSGT

SEQ ID NO: 7 - protein, 379 aa, synthetic construct
MGSSKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGTDYDIPTT

SEQ ID NO: 8 - protein, 220 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 9 - protein, 6 aa, synthetic construct
HHHHHH

SEQ ID NO: 10 - protein, 29 aa, synthetic construct
MWSHPQFEKGGGSGGGSGGSAWSHPQFEK

SEQ ID NO: 11 - protein, 238 aa, synthetic construct
MSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTIQFQDDGKYKTRGEVKFEGDTLVNRIELKGKDFKEDGNILGHKLEYSFNSHNVYIRPDKANNGLEANFKTRHNIEGGGVQLADHYQTNVPLGDGPVLIPINHYLSTQTKISKDRNEARDHMVLLESFSACCHTHGMDELYR

SEQ ID NO: 12 - protein, 7 aa, Tobacco etch virus
ENLYFQS

SEQ ID NO: 13 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 14 - protein, 252 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVE

SEQ ID NO: 15 - protein, 105 aa, Homo sapiens
DVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQ

SEQ ID NO: 16 - protein, 107 aa, Homo sapiens
TNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLP

SEQ ID NO: 17 - protein, 75 aa, Homo sapiens
MLPRPPKVTKEEHFQLHHQMHYSQQITVSLAQGYERTSSPKPRFKSYAYTQAAYVTTSDPTRSPFPSQHLEAPED

SEQ ID NO: 18 - protein, 50 aa, Homo sapiens
AVTTTQPSLTQTTVMETVTTVTTREQILVKHAQEELPPPPPQKKRQITVD

SEQ ID NO: 19 - protein, 47 aa, Homo sapiens
QPDLAPGLTTIGASPTQTVTLVTQPVVTKETAISKLEMPSSLMLEVP

SEQ ID NO: 20 - protein, 72 aa, Homo sapiens
AHRDFGPASQHFLSTSVQGPWERAISPNKVPYYINHETQTTCWDHPKMTELYQSLADLNNVRFSAYRTAMKL

SEQ ID NO: 21 - protein, 110 aa, Homo sapiens
EVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHR

SEQ ID NO: 22 - protein, 109 aa, Homo sapiens
VLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQD

SEQ ID NO: 23 - protein, 110 aa, Homo sapiens
LLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQ

SEQ ID NO: 24 - protein, 111 aa, Homo sapiens
SEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEY

SEQ ID NO: 25 - protein, 106 aa, Homo sapiens
QNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQT

SEQ ID NO: 26 - protein, 108 aa, Homo sapiens
TPLPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEE

SEQ ID NO: 27 - protein, 108 aa, Homo sapiens
MNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKG

SEQ ID NO: 28 - protein, 108 aa, Homo sapiens
LEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEE

SEQ ID NO: 29 - protein, 103 aa, Homo sapiens
WACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQ

SEQ ID NO: 30 - protein, 93 aa, Homo sapiens
IQSAQETEKSLHLIQESLTFIDKQLAAYIADKVDAAQMPQEAQKIQSDLTSHEISLEEMKKHNQGKEAAQRVLSQIDVAQKKLQDVSMKFRLF

SEQ ID NO: 31 - protein, 105 aa, Homo sapiens
PANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEK

SEQ ID NO: 32 - protein, 107 aa, Homo sapiens
LKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLE

SEQ ID NO: 33 - protein, 101 aa, Homo sapiens
QKHMETFDQNVDHITKWIIQADTLLDESEKKKPQQKEDVLKRLKAELNDIRPKVDSTRDQAANLMANRGDHCRKLVEPQISELNHRFAAISHRIKTGKASI

SEQ ID NO: 34 - protein, 93 aa, Homo sapiens
ELEQFNSDIQKLLEPLEAEIQQGVNLKEEDFNKDMNEDNEGTVKELLQRGDNLQQRITDERKREEIKIKQQLLQTKHNALKDLRSQRRKKALE

SEQ ID NO: 35 - protein, 98 aa, Homo sapiens
SHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQ

SEQ ID NO: 36 - protein, 108 aa, Homo sapiens
SYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDR

SEQ ID NO: 37 - protein, 106 aa, Homo sapiens
VEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEE

SEQ ID NO: 38 - protein, 109 aa, Homo sapiens
KNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEA

SEQ ID NO: 39 - protein, 107 aa, Homo sapiens
IKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPD

SEQ ID NO: 40 - protein, 107 aa, Homo sapiens
ALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNE

SEQ ID NO: 41 - protein, 108 aa, Homo sapiens
LKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEE

SEQ ID NO: 42 - protein, 115 aa, Homo sapiens
HRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEA

SEQ ID NO: 43 - protein, 128 aa, Homo sapiens
SDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDW

SEQ ID NO: 44 - protein, 108 aa, Homo sapiens
LERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHE

SEQ ID NO: 45 - protein, 300 aa, Homo sapiens
TSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHY

SEQ ID NO: 46 - protein, 331 aa, Homo sapiens
PMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 47 - protein, 16 aa, synthetic construct
RGVPHIVMVDAYKRYK

SEQ ID NO: 48 - protein, 14 aa, synthetic construct
VPTIVMVDAYKRYK

SEQ ID NO: 49 - protein, 13 aa, synthetic construct
AHIVMVDAYKPTK

SEQ ID NO: 50 - protein, 113 aa, synthetic construct
VDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 51 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 52 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 53 - protein, 1289 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHSYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTMHHHHHHAHIVMVDAYKPTK

SEQ ID NO: 54 - protein, 1289 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHSYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTMAHIVMVDAYKPTKHHHHHH

SEQ ID NO: 55 - protein, 15 aa, synthetic construct
GLNDIFEAQKIEWHE

SEQ ID NO: 56 - protein, 1276 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHSYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTMHHHHHH

(19) United States
(12) Patent Application Publication
(10) Pub. No.: US 2026/0265318 A1
(43) Pub. Date: Sep. 10, 2026

(54) METHOD OF PRODUCING SOLUBLE MICRODYSTROPHIN PROTEIN AND USES OF THE SAME

(71) Applicant: Ultragenyx Pharmaceutical Inc., Novato, CA (US)

(72) Inventors: Jaclyn Cadaoas, Richmond, CA (US); James Lynden Bodnar, Rohnert Park, CA (US); Mark Fox, Greenbrae, CA (US)

(21) Appl. No.: 19/141,400

(22) PCT Filed: Dec. 22, 2023
(86) PCT No.: PCT/US2023/085752

§ 371 Date: Jun. 20, 2025

Related U.S. Application Data
(60) Provisional application No. 63/477,110, filed on Dec. 23, 2022.

(51) Int. Cl. C07K 14/47; C07K 1/14; C07K 1/22; G01N 33/68

(52) U.S. Cl. CPC C07K 14/47; C07K 1/145; C07K 1/22; G01N 33/6854; G01N 33/6893; G01N 2333/47

(57) ABSTRACT

The present disclosure provides novel methods of producing soluble human microdystrophin proteins and uses of the same including for in vitro detection assays.

### CROSS-REFERENCE TO RELATED APPLICATIONS

This application claims the benefit of and priority to U.S. Provisional Patent Application No. 63/477,110, filed Dec. 23, 2022, the entire content of which is hereby incorporated by reference in its entirety for all purposes.

### SEQUENCE LISTING

The instant application contains a Sequence Listing which has been submitted electronically in XML format and is hereby incorporated by reference in its entirety. Said XML copy, created on Dec. 18, 2023, is named ULTA-028WO_SL.xml and is 66,917 bytes in size.

### TECHNICAL FIELD OF THE INVENTION

The present disclosure relates generally to methods of producing soluble recombinant human microdystrophin proteins and uses of the same in, for example, in vitro and/or diagnostic assays.

### BACKGROUND

Muscular dystrophies are a group of monogenic inherited muscle disorders characterized by progressive muscle wasting and weakness. The first gene associated with muscular dystrophy, dystrophin, was cloned by Kunkel et al. in 1987. See Koenig et al., 1987, Cell 50 (3): 509-17 and Kunkel, 2005, Am. J. Hum. Genet. 76:205-14. Mutations in dystrophin are responsible for various forms of muscular dystrophy, including Duchenne muscular dystrophy (DMD), Becker muscular dystrophy, and X-linked dilated cardiomyopathy. DMD is a genetic muscle-wasting disorder that affects approximately 1 in 3500 males. DMD patients generally carry at least one mutation in the dystrophin gene that causes aberrant expression or loss of expression of the dystrophin protein. Patients with DMD experience progressive wasting of skeletal muscles and cardiac dysfunction, which leads to loss of ambulation and premature death, primarily resulting from cardiac or respiratory failure. Unfortunately, currently available treatments are generally only able to slow the progression of DMD.

Wild-type human dystrophin is the largest known human gene, spanning nearly 2.5 million base pairs on the X chromosome and having a coding sequence of about 11.5 kb encoding a protein 3,685 amino acids in length and approximately 426 kDa in size. Because of its size, the dystrophin gene has been difficult to incorporate into suitable gene therapy vectors for clinical testing. Accordingly, researchers have developed various engineered forms of wild-type human dystrophin, in which portions of the wild-type protein are retained, but a large number of the characterized domains of the protein are deleted. These engineered forms are referred to generically as recombinant microdystrophins (or, simply, microdystrophins) because the truncations and/or deletions introduced relative to the wild-type protein render them significantly smaller in size than the wild-type protein.

Even these microdystrophins have proven unwieldy to express and purify in vitro. Efforts to date have not been successful in reproducibly expressing and purifying soluble recombinant human microdystrophin proteins. Thus, there is a need in the art for improved microdystrophins and methods for producing them.

### SUMMARY

The present disclosure provides reliable methods for producing soluble, non-denatured recombinant human microdystrophin (rhMD) proteins, including truncated and engineered versions of human microdystrophins. The methods may involve, for example, expressing and/or purifying the soluble, non-denatured rhMD protein. Also provided are methods of using said rhMD proteins in in vitro, diagnostic, and/or analytical assays.

These methods and characteristics of the provided microdystrophins enable the characterization and use of these microdystrophins in vitro, including in potency assays, immunogenicity assays, and other clinical and diagnostic tool development necessary for evaluation and therapeutic advancement of clinical and pre-clinical engineered human microdystrophin proteins.

In some embodiments, the methods described herein comprise (a) transfecting a host cell culture with a nucleic acid encoding the rhMD protein; (b) expressing the rhMD protein in the host cell culture; (c) generating a cell lysate by incubating the host cell culture with a lysis buffer; (d) centrifuging the cell lysate to generate a cell membrane fraction, said cell membrane fraction comprising plasma membranes of the cell culture; and (e) contacting the cell membrane fraction with a membrane extraction buffer comprising an amount of an anionic detergent to release the rhMD protein from the cell membrane fraction.

In some embodiments, the present disclosure provides a method of producing a recombinant human microdystrophin (rhMD) protein in solution. In some embodiments, the method comprises (a) transfecting a host cell culture with a nucleic acid encoding the rhMD protein; (b) expressing the rhMD protein in the host cell culture; (c) generating a cell lysate by incubating the host cell culture with a lysis buffer; (d) centrifuging the cell lysate to generate a cell membrane fraction, said cell membrane fraction comprising plasma membranes of the cell culture; (e) contacting the cell membrane fraction with a membrane extraction buffer comprising an amount of an anionic detergent to release the rhMD protein from the cell membrane fraction; and purifying the released rhMD protein.

In some embodiments, the methods described herein comprise (a) transfecting a host cell culture with an amount of a nucleic acid encoding the rhMD protein; (b) maintaining the host cell culture for an amount of time for expression of the rhMD protein, wherein the expressed rhMD protein is associated with the cell membrane of the host cells; (c) contacting the host cell culture with a lysis buffer for an amount of time to generate a cell lysate; (d) centrifuging the cell lysate to generate a cell membrane fraction, said cell membrane fraction comprising plasma membranes of the cell culture; and (e) contacting the cell membrane fraction with a membrane extraction buffer comprising an amount of an anionic detergent to release the rhMD protein from the cell membrane fraction.

The methods may comprise (a) contacting the host cell culture with a lysis buffer to generate a cell lysate; (b) centrifuging the cell lysate to generate a cell membrane fraction, said cell membrane fraction comprising plasma membranes of the cell culture; (c) contacting the cell membrane fraction with a membrane extraction buffer comprising an amount of an anionic detergent to release the rhMD protein from the cell membrane fraction; and (d) purifying the released rhMD protein.

Examples of rhMD proteins may comprise an N-terminal actin binding domain (NTD; used interchangeably herein with “ABD1” (actin-binding domain 1)); one to five Spectrin-Like Repeat (R) domains selected from R1 domain, R2 domain, R3 domain, R16 domain, R17 domain, R22 domain, R23 domain, and R24 domain; and a beta-dystroglycan binding domain. The rhMD proteins may comprise one or more Hinge or Hinge-like domains.

For instance, the disclosure provides methods for producing rhMD protein, wherein the rhMD protein comprises five R domains selected from R1 domain, R16 domain, R17 domain, R23 domain, and R24 domain, and a beta-dystroglycan binding domain.

The rhMD protein may further comprise at least a portion of a hinge domain selected from H1 domain, H2 domain, H3 domain, H4 domain and a Hinge like domain.

The rhMD protein may comprise at least a portion of 2 to 3 hinge domains selected from H1 domain, H2 domain, H3 domain, H4 domain and a Hinge like domain.

The rhMD protein may comprise a H1 domain and a H4 domain.

The rhMD protein may comprise an NTD coupled to a H1 domain coupled to a R1 domain coupled to a R16 domain coupled to a R17 domain coupled to a R23 domain coupled to a R24 domain coupled to a H4 domain coupled to a beta-dystroglycan binding domain.

The rhMD protein may comprise an amino acid sequence according to SEQ ID NO: 1 or having at least 90% sequence identity to the amino acid sequence set forth in SEQ ID NO: 1.

The rhMD protein may comprise a sequence according to any one of SEQ ID NO: 2, SEQ ID NO: 3, and SEQ ID NO: 4 or having at least 90% sequence identity to any one of the amino acid sequences set forth in SEQ ID NO: 2, SEQ ID NO: 3, and SEQ ID NO: 4.

In some embodiments, the rhMD protein further comprises a solubility tag selected from any one of: (a) small ubiquitin-like modifier tag (SUMO tag); (b) a Lipoyl domain tag (Lipoyl tag); and (c) a combination thereof, wherein the solubility tag is located at the rhMD protein's N-terminus, at the rhMD protein's C-terminus, or both.

For example, the rhMD protein may comprise at the rhMD protein's N-terminus any one of: (a) a SUMO tag; (b) a Lipoyl tag; and (c) a combination thereof.

Additionally or alternatively, the rhMD protein may comprise at the rhMD protein's C-terminus any one of: (a) a SUMO tag; (b) a Lipoyl tag; and (c) a combination thereof.

In some embodiments, the rhMD protein may comprise a detection tag at the rhMD protein's N-terminus, the rhMD protein's C-terminus, or both the rhMD protein's N- and C-termini, wherein said detection tag is any one of: (a) a maltose binding protein tag (MBP tag); (b) a glutathione S-transferase tag (GST tag); (c) at least one histidine tag (HIS tag); (d) a green fluorescence tag (GFP tag); (e) at least one Strep tag sequence; and (f) a combination thereof.

The solubility tag, the detection tag or both may be linked to the rhMD protein, e.g., by a Tobacco etch virus (TEV) protease cleavage site linker.

In some embodiments, prior to contacting the host cell culture with the lysis buffer, the host cell culture is maintained for an amount of time such that the percentage of live cells (viability) of the host cell culture is any one of: (a) at least 50%; (b) at least 70%; (c) at least 90%; and (d) at least 95%.

The viability of the host cell culture may be determined, e.g., by a microscopy-based assay, colorimetry-based assay, flow-cytometry-based assay, or a combination thereof.

In some embodiments, prior to contacting the host cell culture with the lysis buffer, the host cell culture is maintained for an amount of time such that the percentage of transfected cells in the host cell culture is any one of: (a) at least 50%; (b) at least 70%; (c) at least 90%; and (d) at least 95%.

The percentage of transfected cells may be determined, e.g., by a fluorescence microscopy-based assay, immunostaining-based assay, flow-cytometry-based assay, or a combination thereof.

In some embodiments, the step of expressing the rMD protein in the host cell culture comprises maintaining the host cell culture for an amount of time adequate to express the rhMD protein. In some embodiments, the expressed rhMD protein is associated with the cell membrane of the host cells.

In some embodiments, the step of maintaining comprises incubating the host cell culture for at least 24 hours; at least 48 hours; at least 72 hours; or at least 96 hours.

In some embodiments, the lysis buffer comprises a detergent selected from the group consisting of a non-ionic detergent, a cationic detergent, an anionic detergent, a zwitterionic detergent, and a combination thereof.

The detergent may be, e.g., any one of Triton X-100, Triton X-114, Triton-200, sodium dodecyl sulfate, NP-40, Tween 20, Tween 80, and CHAPS.

In some embodiments, centrifuging of the cell lysate is done at 1100×g to 12000×g for about 5 min to about 1 hour.

In some embodiments, the membrane extraction buffer comprises about 0.2% w/v to about 1.0% w/v of the anionic detergent, such as about 0.3% w/v to about 0.5% w/v of the anionic detergent, or about 0.3% w/v of the anionic detergent.

The anionic detergent may be, e.g., any one of sodium dodecyl sulfate (SDS), sodium deoxycholate (SD), sulfonic acid salt, alcohol sulfate, alkylbenzene sulfonate, phosphoric acid ester, and carboxylic acid salt.

The anionic detergent may be, e.g., any one of ammonium lauryl sulfate, sodium laureth sulfate, sodium lauryl sarcosinate (sarkosyl), potassium lauryl sarcosinate sodium myreth sulfate, sodium pareth sulfate, sodium stearate, sodium lauryl sulfate, a olefin sulfonate, and ammonium laureth sulfate.

In some embodiments, the anionic detergent is sodium lauryl sarcosinate (sarkosyl).

In some embodiments, contacting the cell membrane fraction with the membrane extraction buffer is carried out for about 16 minutes to about 24 minutes.

The methods may further comprise purifying the released rhMD protein using an affinity chromatography resin. For example, the affinity chromatography resin may be, e.g., any one of a reduced-glutathione chromatography resin, a Ni-NTA chromatography resin, and an amylose chromatography resin.

Also provided are uses of the rhMD proteins produced by the described methods, or fragments thereof, in analytical method(s) for detecting a microdystrophin-specific antibody in a sample.

For example, the antibody may be an anti-drug-antibody (ADA) raised in a subject administered a therapeutic microdystrophin gene or protein.

The analytical method may be, e.g., any one of a colorimetric method, enzyme immunoassay (EIA), radioimmunoassay (RIA), fluoroimmunoassay (FIA), chemiluminescent immunoassay (CLIA), and counting immunoassay (CIA).

The analytical method may comprise (a) immobilizing the rhMD protein or a fragment thereof to a solid surface; (b) contacting the immobilized rhMD protein with the sample for an amount of time sufficient for binding of the immobilized rhMD protein to the microdystrophin-specific antibody, if present in the sample, to form a bipartite complex of the rhMD protein and the microdystrophin-specific antibody; (c) contacting the bipartite complex with a detection reagent that binds specifically to the microdystrophin-specific antibody and not the rhMD protein, for an amount of time to form a tripartite complex of the detection reagent, the rhMD protein, and the dystrophin-specific antibody, wherein the detection reagent comprises a detectable moiety that generates a detectable signal; and (d) detecting the signal generated in step (c), wherein presence of the detectable signal indicates presence of microdystrophin-specific antibody in the sample, and absence of the detectable signal indicates absence of microdystrophin-specific antibody in the sample.

The rhMD protein or fragment thereof may be immobilized to the solid surface, e.g., by any one of: a) an anti-microdystrophin antibody bound to the solid surface; and b) covalent crosslinking to the solid surface.

The solid surface may be, e.g., any one of polyethylene, polyacrylamide, polystyrene, agarose, glass, and silicone rubber.

The analytical method may further comprise i) contacting the immobilized rhMD protein of step (a) with a coating/blocking buffer before proceeding with step (b); ii) contacting the bipartite complex of step (b) with a wash buffer before proceeding with step (c); iii) contacting the tripartite complex of step (c) with a wash buffer before proceeding with step (d); iv) contacting the tripartite complex of step (c) with a detection solution before proceeding with step (d); or v) a combination thereof.

The detectable moiety may be, e.g., any one of an enzyme, a fluorophore, and a radioisotope.

The detectable signal may be, e.g., any one of a colorimetric signal, a fluorescent signal, and radiation.

Where the detectable moiety is an enzyme, step (c) may further comprise contacting the tripartite complex with a substrate for generating the detectable signal.

The enzyme may be, e.g., any one of a horse radish peroxidase, an alkaline phosphatase, a galactosidase, an acetylcholinesterase, and a catalase.

The fluorophore may be, e.g., any one of fluorescein isothiocyanate (FITC), phycoerythrin (PE) or a conjugate thereof, an alexa fluor dye, a rhodamine dye, a Cy-based dye, a G-dye, and Texas red.

The radioisotope may be, e.g., any one of ⁹⁰Y, ¹¹¹In, ¹⁷⁷Lu, ^{99m}Tc, ¹²³I, ¹²⁵I, and ¹³¹I.

For example, the detection reagent may be an antibody or a fragment thereof such as any one of an anti-Dystrophin antibody, clone 2C6 (MANDYS106), Dystrophin B (Dys B), Dystrophin antibody, DY4/6D3 clone (DYS1-CE), Dystrophin antibody, DY8/6C5 clone (DYS2-CE), and Dystrophin antibody, DY10/12B2 clone (DYS3-CE) antibody.

Also provided are uses of rhMD protein produced by the methods described herein, or fragments thereof, in an analytical method of quantitatively measuring an amount of microdystrophin present in a sample solution.

The method of quantitatively measuring an amount of microdystrophin may be, e.g., any one of a colorimetric method, enzyme immunoassay (EIA), radioimmunoassay (RIA), fluoroimmunoassay (FIA), chemiluminescent immunoassay (CLIA), and counting immunoassay (CIA).

The analytical method may comprise: (a) contacting the sample solution with a microdystrophin capture reagent and a microdystrophin detection reagent, and detecting a sample signal from the microdystrophin detection reagent in the sample solution; (b) measuring the sample signal; (c) contacting one or more reference solutions, each comprising a known amount of the rhMD protein, with a microdystrophin capture reagent and a microdystrophin detection reagent, and detecting one or more comparator signals from the microdystrophin detection reagent in each of the one or more solutions; (d) measuring the one or more comparator signals; and (e) comparing the sample signal with the one or more comparator signals to quantitatively determine the concentration of microdystrophin in the sample solution.

The one or more reference solutions may be, e.g., dilutions of a standard solution and may comprise from about 0.01 ng/ml to about 50 ng/ml of rhMD protein, wherein the standard solution comprises a known concentration of the rhMD protein produced according to the methods described herein, or a fragment thereof.

The microdystrophin capture reagent may have binding specificity for a microdystrophin in the sample solution and for the rhMD protein or a fragment thereof in the standard solution.

The microdystrophin capture reagent may be, e.g., an antibody or a fragment thereof.

The microdystrophin capture reagent may be, e.g., immobilized to a solid surface.

The concentration of the microdystrophin capture reagent may be, e.g., from about 0.5 mg/ml to about 5 mg/ml.

The microdystrophin capture reagent may be immobilized, e.g., by protein A to a solid surface.

The microdystrophin detection reagent may bind specifically to a microdystrophin in the sample solution and the rhMD protein or a fragment thereof in the standard solution but not to the microdystrophin capture reagent.

The microdystrophin detection reagent may be, e.g., an antibody or a fragment thereof.

The microdystrophin detection reagent may be present, e.g., at a concentration of from about 0.5 ng/ml to about 2 mg/ml during step (a).

The microdystrophin capture reagent may be, e.g., any one of MANDYS106, Dys B, DYS1-CE, DYS2-CE, and DYS3-CE antibody.

In some embodiments, the microdystrophin detection reagent is any one of Dys B, MANDYS106, DYS1-CE, DYS2-CE, and DYS3-CE antibody, and wherein the microdystrophin capture reagent is different from the microdystrophin detection reagent.

In some embodiments, detecting a signal from the microdystrophin detection reagent comprises contacting the microdystrophin detection reagent with a secondary detection reagent that specifically binds the microdystrophin detection reagent and not the microdystrophin capture reagent.

The secondary detection reagent may be, e.g., an antibody or a fragment thereof.

The secondary detection reagent may comprise, e.g., a reporter moiety.

The reporter moiety may be, e.g., any one of a horse radish peroxidase, an alkaline phosphatase, a galactosidase, an acetylcholinesterase, and a catalase.

In some embodiments, the analytical (e.g., quantitative measurement) method may further comprise contacting the sample solution and the one or more reference solutions comprising known amounts of the rhMD protein with a washing solution before measuring the sample signal.

The sample utilized in any of the analytical methods described herein may be, e.g., from a subject suffering from or suspected of having a dystrophinopathy. In some embodiments, the subject has been administered or is to be administered a therapeutic microdystrophin gene or protein.

Also provided herein are kits for detecting microdystrophin specific antibody in a sample, comprising: (a) a recombinant human microdystrophin (rhMD) protein produced by the methods herein or a fragment thereof; and (b) a detection reagent that specifically binds to the microdystrophin specific antibody but not to the rhMD protein or fragment thereof, wherein the detection reagent comprises a detectable moiety.

The kit may comprise: a solid surface; a coating/blocking solution; a washing solution; a detection solution; or a combination thereof.

In some embodiments, the rhMD protein or fragment thereof is immobilized to the solid surface.

The kit may further comprise an anti-microdystrophin antibody or a crosslinker for immobilizing the rhMD protein or fragment thereof to the solid surface.

The solid surface may be coated, e.g., with protein A agarose.

The solid surface may be, e.g., any one of polyethylene, polyacrylamide, polystyrene, agarose, glass, and silicone rubber.

The detectable moiety may be, e.g., any one of an enzyme, a fluorophore, and a radioisotope.

The detectable moiety may generate a detectable signal, e.g., any one of a colorimetric signal, a fluorescent signal, and radiation.

The detectable moiety may be, e.g., an enzyme, and the kit may further comprise a substrate that generates a detectable signal upon reacting with the enzyme.

The enzyme may be, e.g., any one of a horse radish peroxidase, an alkaline phosphatase, a galactosidase, an acetylcholinesterase, and a catalase.

The fluorophore may be, e.g., any one of fluorescein isothiocyanate (FITC), phycoerythrin (PE) or a conjugate thereof, an alexa fluor dye, a rhodamine dye, a Cy-based dye, a G-dye, and Texas red.

The radioisotope may be, e.g., any one of ⁹⁰Y, ¹¹¹In, ¹⁷⁷Lu, ^{99m}Tc, ¹²³I, ¹²⁵I and ¹³¹I.

The detection reagent may be, e.g., an antibody or a fragment thereof, such as any one of MANDYS106, Dys B, DYS1-CE, DYS2-CE, and DYS3-CE antibody.

Also provided are kits for quantitatively measuring an amount of microdystrophin present in a sample solution comprising: (a) a microdystrophin capture reagent; (b) a microdystrophin detection reagent; and (c) a standard solution comprising a recombinant human microdystrophin (rhMD) protein produced by the methods described herein, or a fragment thereof, or a lyophilized reagent for preparing said standard solution.

In some embodiments, the microdystrophin detection reagent binds specifically to microdystrophin in the sample and the rhMD protein or a fragment thereof in the standard solution.

The microdystrophin detection reagent may be, e.g., an antibody or a fragment thereof.

The kits may further comprise: a solid surface; a coating solution; a washing solution; a detection solution; or a combination thereof.

The microdystrophin capture reagent may bind specifically to microdystrophin in the sample and the rhMD protein or a fragment thereof in the standard solution.

In some embodiments, the microdystrophin capture reagent is an antibody or a fragment thereof.

The microdystrophin capture reagent may be, e.g., immobilized to the solid surface.

The microdystrophin capture reagent may be, e.g., immobilized by a protein A to the solid surface.

The concentration of the microdystrophin capture reagent may be, e.g., from about 0.5 mg/ml to about 5 mg/ml.

The microdystrophin detection reagent may bind specifically to microdystrophin in the sample and the rhMD protein or a fragment thereof in the standard solution but not to the microdystrophin capture reagent.

The microdystrophin detection reagent may be, e.g., an antibody or a fragment thereof.

The microdystrophin detection reagent may be, e.g., used at a dilution of about 0.5 mg/ml to about 2 mg/ml.

The microdystrophin capture reagent may be, e.g., any one of a Dys B, MANDYS106, Dys B, DYS1-CE, DYS2-CE, and DYS3-CE antibody.

In some embodiments, the microdystrophin detection reagent may be, e.g., any one of a Dys B, MANDYS106, DYS1-CE, DYS2-CE, and DYS3-CE antibody, and the microdystrophin detection reagent is different from the microdystrophin capture reagent.

The kits may further comprise a secondary detection reagent that specifically binds the microdystrophin detection reagent and not the microdystrophin capture reagent.

The secondary detection reagent may be, e.g., an antibody or a fragment thereof.

In some embodiments, the secondary detection reagent comprises a reporter moiety.

The reporter moiety may be, e.g. any one of a horse radish peroxidase, an alkaline phosphatase, a galactosidase, an acetylcholinesterase, and a catalase.

Also provided are recombinant human microdystrophin (rhMD) proteins produced according to the methods described herein, wherein the rhMD protein comprises an N terminal actin binding domain (NTD); at least a portion of 2 or 3 Hinge (H) domains selected from H1 domain, H2 domain, H3 domain, H4 domain, and a Hinge-like domain; one to five Spectrin-Like Repeat (R) domains selected from R1 domain, R2 domain, R3 domain, R16 domain, R17 domain, R22 domain, R23 domain, and R24 domain; and a beta-dystroglycan binding domain in solution.

The rhMD protein may consist essentially of an NTD coupled to a H1 domain coupled to a R1 domain coupled to a R16 domain coupled to a R17 domain coupled to a R23 domain coupled to a R24 domain coupled to a H4 domain coupled to a beta-dystroglycan binding domain.

These and other aspects and features are described in the following sections of the present disclosure.

### BRIEF DESCRIPTION OF THE DRAWINGS

The invention can be more completely understood with reference to the following drawings.

FIGS. 1A-1D show illustrative diagrams depicting structural and functional domains of a full-length human dystrophin protein and an exemplary recombinant human microdystrophin (rhMD) protein. FIGS. 1A-1B depict spatial arrangement of the N-terminal actin binding domain (ABD1), R domains (R), Hinge domains (H), Cysteine rich domain (CR) and carboxy-terminal coiled repeat domain (CT), as indicated within the full-length dystrophin and rhMD proteins, respectively. FIG. 1C depicts the functionalities retained by the rhMD with respect to the various domains. FIG. 1D depicts the retained domains of rhMD protein which allow it to anchor to the cell membrane.

FIGS. 2A-2B show illustrative diagrams depicting alternative configurations of rhMD proteins having solubility, detection, and/or affinity tags. FIG. 2A shows various configurations of solubility, detection, and affinity tags that can be added to rhMD proteins.

FIG. 2B is a diagram showing the Tobacco Etch Virus (TEV) linker, solubility tags (SUMO and Lipoyl) and affinity/detection tags (TwinStrep), maltose binding protein tag (MBP), glutathione-S-transferase tag (GST) and histidine tag (HIS)) linked to the rhMD protein of the disclosure, as indicated.

FIGS. 3A-3E depict the transfection optimization of a plasmid DNA construct encoding the rhMD of the present disclosure. FIG. 3A is a diagram depicting the rhMD protein linked to a SUMO-TEV tag at the N-terminus and the TEV-GFP-TwinStrep tag at the C-terminus, as indicated. The different domains of the rhMD protein are as indicated. FIGS. 3B-3C are graphs showing the percentage viability and transfection efficiency, respectively, of Expi293 cells transfected with increasing doses of the plasmid construct encoding the rhMD protein of FIG. 3A with respect to increasing time points of incubation post-transfection. FIG. 3D depicts 40× Mag fluorescence microscopy images showing expression of the rhMD protein in Expi293 cells transfected with plasmid construct encoding the rhMD protein (upper left panel), a GFP encoding plasmid transfected positive control (upper right panel) and untransfected (negative control; lower panel), at 48 hours post-transfection, as indicated. FIG. 3E depicts an SDS-PAGE image showing the presence of the rhMD protein in lysate derived from Expi293 cells transfected with plasmid construct encoding the rhMD protein of FIG. 3A.

FIGS. 4A-4D show insolubility of the rhMD produced from cells transfected with rhMD encoding plasmid construct. FIG. 4A is a diagram depicting the rhMD protein linked to a Lipoyl-TEV tag at the N-terminus and the TEV-MBP tag at the C-terminus, as indicated. The different domains of the rhMD protein are as indicated. FIG. 4B is an SDS PAGE gel image depicting the relative levels of rhMD protein in the whole cell extract (whole cell lysate) and soluble fraction (supernatant obtained by lysing the cells by sonication and then centrifuging the whole cell extract) prepared from Expi293 cells transfected with rhMD protein encoding plasmid construct (encoding the rhMD protein 1 depicted in FIG. 4A) for 48 hours post-transfection. FIG. 4C is a MBPTrap affinity chromatography step elution profile of the rhMD protein purified from the soluble fraction obtained prepared from Expi293 cells transfected with rhMD protein encoding plasmid construct (FIG. 4A) for 48 hours post-transfection. FIG. 4D is an SDS PAGE gel image depicting yield of the rhMD protein (depicted in FIG. 4A) in pooled eluents of the MBPTrap affinity chromatography run of FIG. 4C.

FIGS. 5A-5B show that alkaline extraction is not efficient in increasing the solubility of membrane-bound protein fraction from cells expressing the rhMD of the present disclosure. FIG. 5A is an SDS-PAGE gel image depicting the level of rhMD protein (depicted in FIG. 4A) in whole cell lysates and soluble fraction (supernatant obtained by centrifuging the whole cell extract) prepared from Expi293 cells transfected with rhMD encoding plasmid. FIG. 5B is a SDS PAGE gel image depicting the level of rhMD protein (depicted in FIG. 4A) obtained from rhMD expressing Expi293 cells, in whole cell lysate (CL); cell lysate post-sonication of expressing cells (L); supernatant prepared by centrifugation at 3000 rpm of the post-sonication cell lysate (S1); resuspended cell pellets in alkaline extraction buffer, at pH 11 (PA); resuspended cell pellets in alkaline extraction buffer at pH 11, post 1 hr incubation (PB); S1 supernatant post 1 hr incubation (SB) (supernatant prepared by centrifugation at 3000 rpm of the resuspended cell pellets); post 1 hr incubation in alkaline extraction buffer (S2); and resuspended cell pellet post centrifugation at 3000 rpm (P2) as indicated (pH 11).

FIGS. 6A-6B show the level of rhMD protein (depicted in FIG. 4A) obtained from rhMD expressing Expi293 cells by extraction using cationic, anionic, or zwitterionic detergents. FIG. 6A is an SDS PAGE gel image showing the level of rhMD protein in the resuspended pellet (P1) obtained from a post-sonification lysate (L) mixed with either 25 mM Tris with 100 mM NaCl, 1% Triton X-100, 1% sarkosyl, or 1% CHAPS detergent, and in the supernatant (S2) and cell pellet (P2) obtained by centrifugation of the resuspended pellet, P1, at 3000 rpm for 10 minutes. FIG. 6B is a SDS PAGE gel image showing the level of rhMD protein in the resuspended pellet (P1) obtained from a post-sonification lysate (L) mixed with either 0.2%, 0.3%, 0.4% or 0.5% sarkosyl and in the supernatant (S2) and cell pellet (P2) obtained by centrifuging the resuspended pellet, P1, at 3000 rpm for 10 minutes.

FIG. 7A-7B are illustrations showing the strategy for determining effect of anionic detergent-based extraction on solubility of different rhMD proteins. FIG. 7A is an illustration showing the structural domains of four different human microdystrophin proteins that were evaluated for effect of anionic detergent-based extraction. The name of each microdystrophin protein and the specific structural domains deleted in each microdystrophin protein are as indicated. Each microdystrophin protein is linked to a N-terminus Lipoyl-TEV-tag and a C-terminus HIS-TEV-tag. FIG. 7B is a flow chart depicting the details and steps of anionic detergent based solubilization protocol of the Lipoyl-TEV- and HIS-TEV-tagged microdystrophin proteins. The specific cell lysate preparation, solubilization/membrane extraction and affinity purification steps are as indicated. Buffers used were as follows: Lysis buffer (25 mM Tris, 500 mM NaCl, pH 8+protease inhibitors), solubilization buffer (25 mM Tris, 500 mM NaCl, 0.5% sarkosyl pH 8, Buffer A1 (EQ): 25 mM Tris, 500 mM NaCl and 0.5% sarkosyl pH 8), Buffer A3 (25 mM Tris, 500 mM NaCl, 0.05% sarkosyl, pH 8) and Buffer B1 (25 mM Tris, 500 mM NaCl, 0.05% sarkosyl, 500 mM imidazole pH 8).

FIGS. 8A-8I show effect of anionic detergent based solubilization and extraction of four rhMD proteins linked to a detection tag. FIGS. 8A, 8C, 8E, and 8G depict Immobilized Metal Affinity Chromatography (IMAC) affinity chromatography step elution profiles of the rhMD proteins 1, 2, 3, and 4, respectively, of FIG. 7A, linked with a C-terminal HIS tag. The rhMD proteins 1, 2, 3, and 4 were expressed in Expi293 cells transfected with the respective rhMD protein-encoding plasmid construct, harvested at 48 hours post-transfection, solubilized and purified according to the protocol of FIG. 7B, and purified from the soluble fractions so generated, using a HisTrap Excel affinity chromatography column. The specific rhMD protein yield and total pooled fractions collected are as indicated. FIGS. 8B, 8D, 8F, and 8H are SDS PAGE gel images depicting the yield of rhMD proteins 1, 2, 3, and 4 linked with only a C-terminal HIS tag, from the extraction of eluent fractions of the respective IMAC affinity chromatography runs. Lanes corresponding to the whole cell lysate (WC), lysate post sonication (CL), supernatant (S1) and pellet (P1) produced by centrifuging CL at 3000 rpm, supernatant (S2) and insoluble pellet (P2) following solubilization of P1, and each eluent fraction from the respective IMAC affinity chromatography runs, are as indicated. FIG. 8I is an SDS-PAGE gel comparing the levels of each of the four micro-dystrophin proteins, present in lysate post-sonication (CL), supernatant (S1) and pellet (P1) produced by centrifuging CL at 3000 rpm, supernatant (S2) and insoluble pellet (P2) following solubilization of P1, and pooled eluent fraction from the eluent fractions of the respective IMAC affinity chromatography runs, as indicated.

FIGS. 9A-9I show effect of solubility tag linkage and anionic detergent based solubilization and extraction of four rhMD proteins. FIGS. 9A, 9C, 9E, and 9G depict IMAC affinity chromatography step elution profiles of the rhMD proteins 1, 2, 3, and 4 of FIG. 7A, linked with an N-terminal TEV-lipolyl tag and a C-terminal TEV-HIS tag, expressed in Expi293 cells transfected with the respective rhMD protein-encoding plasmid construct, harvested at 48 hours post-transfection, using the solubilization and purification protocol of FIG. 7B, and purified from a HisTrap Excel affinity chromatography column. The specific rhMD protein yield and total pooled fractions collected are as indicated. FIGS. 9B, 9D, 9F, and 9H are SDS-PAGE images depicting yield of the rhMD proteins linked with an N-terminal Lipoyl-TEV tag and a C-terminal TEV-HIS tag, from sarkosyl extraction of eluent fractions of the respective IMAC affinity chromatography runs. Lanes corresponding to the whole cell lysate (WC), lysate post sonication (CL), supernatant (S1) and pellet (P1) produced by centrifugating CL at 3000 rpm, supernatant (S2) and insoluble pellet (P2) following solubilization of P1, and each eluent fraction from the respective IMAC affinity chromatography runs, are as indicated. FIG. 9I is a SDS-PAGE image comparing the levels of each of the four rhMD proteins linked with either an N-terminal Lipoyl-TEV tag and a C-terminal TEV-HIS tag or linked with only a C-terminal HIS tag (FIG. 9I), present in lysate post sonication (CL), supernatant (S1) and pellet (P1) produced by centrifugating CL at 3000 rpm, supernatant (S2) and insoluble pellet (P2) following solubilization of P1, and pooled eluent fraction from the eluent fractions of the respective IMAC affinity chromatography runs, as indicated.

FIGS. 10A-10C show the stability of the purified rhMD protein based on detection by a dystrophin specific antibody. FIG. 10A is a diagram depicting the enzyme linked immunosorbent assay (ELISA) for detection of the purified rhMD protein. The immobilized capture antibody (MANDYS106), detection antibody (DysB ANTIBODY) and secondary antibody (SULFO-TAG LABELED ANTI-MOUSE IgG1) are as indicated. FIG. 10B is a representative chart showing a dose dependent detection of the expressed and purified rhMD protein specifically by uDys detection antibody (DysB) and not any β-Dys detection antibodies (MANDAG2 and ProTech), as indicated. Error bars indicate standard deviation. FIG. 10C is a chart showing linear relationship between the amount of purified rhMD protein and detection signal generated by the ELISA method using DysB, MANDAG2 and Protech detection antibodies of FIG. 10B.

FIG. 11 shows the amino acid sequence of rhMD protein 1 of SEQ ID NO: 1 with a C-terminal HIS tag (HHHHHH, SEQ ID NO: 9)) (SEQ ID NO: 56) in 1-letter code as evaluated by peptide mapping. Matched peptides following an enzymatic digestion with a mixture of trypsin and chymotrypsin are bolded. Non-bolded residues indicate positions that could not be confirmed by peptide mapping. Long dashed underlines represent individual digest fragments from trypsin proteolysis. Alternating long and short dash underlines represent individual digest fragments from elastase proteolysis. Dotted underlines represent individual digest fragments from chymotrypsin proteolysis. Solid underlines represent individual digest fragments from AspN proteolysis.

FIG. 12A shows the rod-like structure of rhMD protein 1 of SEQ ID NO: 1 having a very large predicted hydrodynamic radius. FIG. 12B shows SEC HPLC results, demonstrating hypothesized monomeric and dimeric forms of rhMD protein 1 formulated in 0.05% sarkosyl.

FIG. 13 shows SEC HPLC results, demonstrating hypothesized monomeric form of rhMD protein 1 formulated in 0.5% sarkosyl.

FIG. 14A through FIG. 14G show SEC HPLC results for rhMD protein 1 formulated in reduced amounts of sarkosyl from 0.5% down to 0.025% sarkosyl.

### DETAILED DESCRIPTION

All publications, patents, and patent applications, including any drawings and appendices therein, are incorporated by reference in their entirety for all purposes to the same extent as if each individual publication, patent or patent application, drawing, or appendix was specifically and individually indicated to be incorporated by reference in its entirety for all purposes.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the present specification, where an element or component is said to be included in and/or selected from a list of recited elements or components, it should be understood that the element or component can be any one of the recited elements or components, or the element or component can be selected from a group consisting of two or more of the recited elements or components.

Throughout the present specification, numerical ranges are provided for certain quantities. It is to be understood that these ranges comprise endpoints and all subranges therein, including each integer in and between the endpoints of a disclosed range. Thus, the range “from 50 to 80” includes all possible ranges therein (e.g., 51-79, 52-78, 53-77, 54-76, 55-75, 60-70, etc.) as well as each individual integer from 50 to 80 (e.g., 50, 51, 52, 53, 54, etc.). Where ranges are provided in the form or fractions, percentages, decimals, and the like, such ranges likewise include the endpoints and all possible subranges therein and each individual fraction, percentage, decimal, etc. in and between the endpoints of disclosed range. For example, the range “from 0.1 to 1.0” includes all possible ranges therein (e.g., 0.2 to 0.9, etc.) and each individual 1/10^{th} decimal from 0.1 to 1.0 (e.g., 0.1, 0.2, 0.3, 0.4, etc.). Furthermore, all values within a given range may be an endpoint for the range encompassed thereby (e.g., the range 50-80 includes the ranges with endpoints such as 55-80, 50-75, etc.).

It should be understood that the order of steps or order for performing certain actions is immaterial so long as the presently described methods remain operable. Moreover, two or more steps or actions may be conducted simultaneously.

The use of any and all examples, or exemplary language herein, for example, “such as” or “including” is intended merely to illustrate better the present invention and does not pose a limitation on the scope of the invention unless claimed. No language in the specification should be construed as indicating any non-claimed element as essential to the practice of the present invention.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within the present disclosure, embodiments have been described and depicted in a way that enables a clear and concise disclosure to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

### Definitions

The following terms used in the present specification and claims are believed to be well understood by one of ordinary skill in the art given their use and context herein. The following definitions are set forth to facilitate explanation of the presently disclosed subject matter.

The term “a” or “an” refers to one or more of that entity; for example, “a microdystrophin protein” refers to one or more microdystrophin proteins or at least one microdystrophin protein unless context dictates otherwise. As such, the terms “a” (or “an”), “one or more” and “at least one” are used interchangeably herein unless context dictates otherwise. In addition, reference to, e.g., “an agent” by the indefinite article “a” or “an” does not exclude the possibility that more than one of agents is present, unless the context clearly requires that there is one and only one agent present.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The term “affinity tag” refers to peptide domains that can be added to recombinantly expressed proteins (i.e., “fusion proteins”) to aid in the separation, isolation, and/or purification of the recombinant protein. Typically, affinity tags comprise an epitope or interaction domain that can be specifically recognized by a known antibody or that interacts with high affinity to a known reagent or material. A chromatography resin or other solid substrate can then be prepared with the antibody or reagent/material immobilized thereon, creating a substrate for the separating, isolating, and/or purifying of the recombinant protein comprising the affinity tag. Examples of affinity tags suitable for use in the recombinant proteins and methods disclosed herein include but are not limited to poly-histidine (e.g., 6×His, SEQ ID NO: 9) tags, S-tag, glutathione-S-transferase (GST), streptavidin and variants thereof, protein A, FLAG peptides, and maltose-binding protein (MBP) tags.

The term “cleavage site” refers to peptide domains or chemical linkers between domains of recombinantly expressed proteins that can be specifically cleaved by defined conditions, chemical, or enzymatic cleavage. Such cleavage sites enable removal of, e.g., affinity tags or solubility tags or other domains fused to the recombinant proteins as expressed. Examples of protein cleavage sites suitable for use in the recombinant proteins and methods disclosed herein include but are not limited to peptide cleavage sites, disulfide cleavage sites, peptide sites recognized by intracellular proteases (lysosomal cleavage sites), and Tobacco etch virus cleavage site.

The term “detection tag” refers to peptide domains that can be added to recombinantly expressed proteins (i.e., fusion proteins) to aid in the detection of the fusion protein, e.g., expressed in a cell or tissue or a present in sample. Detection tags include fluorescence-based and immunoaffinity based tags which can be detected in a tissue or a sample using, e.g., light or fluorescence microscopy and/or immunoaffinity-based cross-linking and detection. In some instances, detection tags can be used as affinity tags, and vice versa.

The term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

The term “lysis buffer” refers to a solution suitable for use in lysing cells or disrupting cell membranes to facilitate separation of cytosolic and membrane-based fractions of the cells. For example, a lysis buffer may be used to disrupt (lyse, break, tear, or destabilize) cellular membranes of cells present in a cell culture, enabling separation of a cytosolic fraction (supernatant) and membrane fraction (pellet) by centrifugation of the lysed cells. As described further herein, lysis buffers may comprise a detergent, e.g., a cationic detergent, an anionic detergent, or a zwitterionic detergent.

The term “membrane extraction buffer” refers to a solution suitable for use in extracting proteins from membrane fractions of lysed cells. For example, a membrane extraction buffer may be used to disrupt (i.e., dissolve or resuspend) a membrane fraction pellet resulting from the separation of cytosolic fraction (supernatant) and membrane fraction (pellet) by centrifugation of the lysed cells. As described further herein, membrane extraction buffers may comprise a detergent, e.g. an anionic detergent.

The term “non-denatured” as it relates to proteins refers to the 3-dimensional structure and conformation of a protein as it exists in a cellular environment, cellular milieux, and/or extracellular environment/milieux. That is, non-denatured refers to a protein as it exists in its physiological state, including secondary, tertiary, and quaternary folding structure and binding/interaction partners. Often, denaturation steps are implemented to detect or isolate expressed proteins. Such denaturation steps may include cleavage of disulfide bonds, covalent cross-linking, stripping of glycosylation and other post-translational modifications, and/or disruption of non-covalent interactions such as intra-protein interactions between protein domains and/or interactions between proteins (such homomultimeric and/or heteromultimeric interactions). Thus, non-denatured refers to a protein that retains these structural, conformational, and intra- and inter-protein interaction characteristics. Non-denatured proteins may retain some or all of the structural, conformational, and intra- and inter-protein interaction characteristics of the expressed protein as it exists in a cellular environment, cellular milieux, and/or extracellular environment/milieux.

The term “recombinant human microdystrophin” (rhMD) protein includes engineered, truncated, and otherwise modified variants of a wild type or native dystrophin protein. rhMD proteins (referred to at times throughout the specification simply as rhMDs) are non-naturally occurring recombinant polypeptides and are generally designed to reduce the molecular size/weight of a dystrophin polypeptide while retaining certain advantageous or important biological functions of wild type or native dystrophin. Generally speaking, the wild type or native dystrophin protein refers to the human polypeptide sequence of dystrophin, consisting of 3685 amino acid residues (SEQ ID NO: 13) (canonical human dystrophin protein sequence, UniProt ID no. P11532, encoded by NCBI RefSeq no. NM_004006.3).

The term “soluble” or “solubilized” or “released” and the like, as used in the context of expressed proteins, refers to the status of the protein in a liquid buffer to form a solution with that buffer. If the protein is soluble, it is in solution or it can be solubilized into solution or released from interactions with other proteins/components to be in solution in that buffer. Cells can be separated into cytosolic and membrane fractions. Proteins expressed in cells (intracellular proteins) may be associated with the cytosol or may be associated with a membrane. Proteins that are membrane-endogenous, such as transmembrane proteins and other proteins that are fully or partially embedded in membranes or otherwise strongly interact with membranes, may be difficult to solubilize for purification. The process of extracting such proteins from membranes is referred to as solubilization. A protein so extracted may be referred to as “released” from the membrane.

The term “solubility tag” refers to a domain that can be added to recombinantly expressed proteins (i.e., fusion proteins) and that serve to increase the solubility of the recombinant protein. Without wishing to be bound by theory, solubility tags may function to destabilize protein interactions by promoting proper folding, increasing translation initiation of the recombinant protein, and/or providing polypeptide chains rich in positively charged amino acids that increase electrostatic repulsion during translation reducing/avoiding aggregation. Examples of solubility tags suitable for use in the recombinant proteins and methods described herein include the small ubiquitin-like modifier tag (SUMO tag) and the Lipoyl domain tag (Lipoyl tag), among others such as maltose-binding protein tag (MBP tag), glutathionine-S-transferase tag (GST tag), thioredoxin tag (Trx tag), N-utilization substance tag (NusA tag), and green fluorescence protein (GFP). In some instances, solubility tags may double as affinity tags.

### INTRODUCTION

The present disclosure provides a reliable method for producing soluble, non-denatured recombinant human microdystrophin (rhMD) protein. Various rhMDs are currently in pre-clinical and clinical stage testing for safety and efficacy in treating dystrophinopathies. (See, for example, ClinicalTrials.gov Identifiers: NCT03368742, NCT03362502, NCT05429372, NCT03769116, NCT04626674, NCT05096221, NCT04281485, and NCT05096221). These therapeutic proteins are typically delivered to muscle tissue using viral gene therapy vectors comprising a vector genome encoding the recombinant human microdystrophin protein. For example, multiple candidate AAV gene therapies are being evaluated for the ability to deliver functional recombinant human microdystrophin protein to muscle tissue of patients suffering from DMD.

A critical aspect of demonstrating efficacy of such therapies is showing that administration of the gene therapy vectors to a subject results in meaningful expression of the rhMD protein in muscle tissue of the subject. The obverse is also true. It is important to show what level of expression is required in muscle tissue to establish efficacy in a subject. Further, like the wild-type dystrophin protein that DMD patients lack, the rhMD should be localized to the cell membrane and should interact with proper functional binding partners of dystrophin, i.e., actin, nNos, syntrophin, and beta-dystroglycan. Further, it will be important to demonstrate the levels of such proteins present in muscle tissue of subjects administered rhMD-based therapeutics, including gene therapies. Accordingly, quantitative methods of measuring rhMD protein present in subject samples are needed.

Previous attempts to develop such methods have been hampered by the difficulty of reliably expressing and purifying rhMD protein in vitro because a source of such rhMD protein is needed to calibrate quantitative assays. The presently disclosed methods thus enable quantitative measurement of rhMD protein present in samples, e.g., from patient muscle tissue, by providing a source of recombinant protein that can be used as a comparator and to generate a calibration curve in quantitative assays.

The presently disclosed methods also enable further in vitro characterization of the therapeutic rhMD proteins to better understand their function, their intracellular trafficking, and their binding/interaction partners.

Another critical aspect of evaluating efficacy and safety of rhMD-based therapeutics, including viral vector-mediated rhMDs, is the ability to detect antibodies in patients that react with the rhMD proteins. Immune responses in some subjects administered therapeutic drugs, including proteins and therapeutic transgenes, for instance, are known to give rise to anti-drug antibodies (ADAs). This immune response can curtail the efficacy of a therapy over time in a subject, and can also reduce the efficacy of the same therapy upon re-administration. Accordingly, methods for detecting ADAs in subjects administered rhMD-based therapeutics as well as in subjects to be administered such therapeutics are needed. However, the lack of reliable methods for producing soluble, non-denatured rhMD protein has made it difficult to develop these methods. Thus, the presently disclosed methods also enable assays for detecting anti-microdystrophin antibodies in samples from subjects.

The presently disclosed methods achieve these and other ends by providing rhMD protein examples and assays for producing and purifying the same that result in reliable expression of recombinant human microdystrophin proteins that are soluble and non-denatured.

Recombinant Human Microdystrophin (rhMD) Proteins

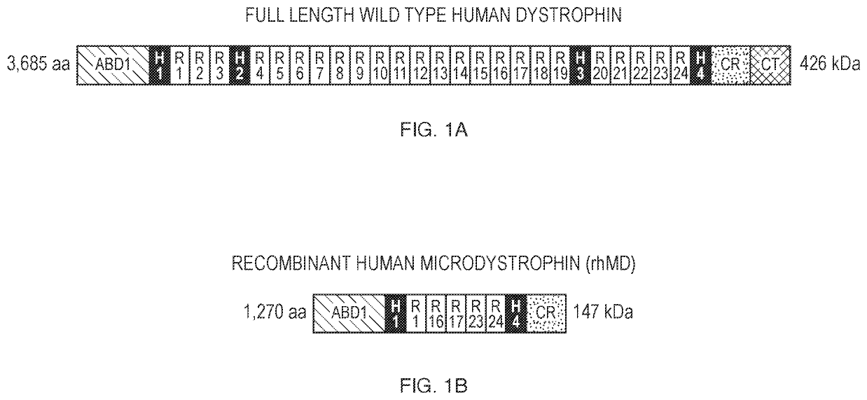

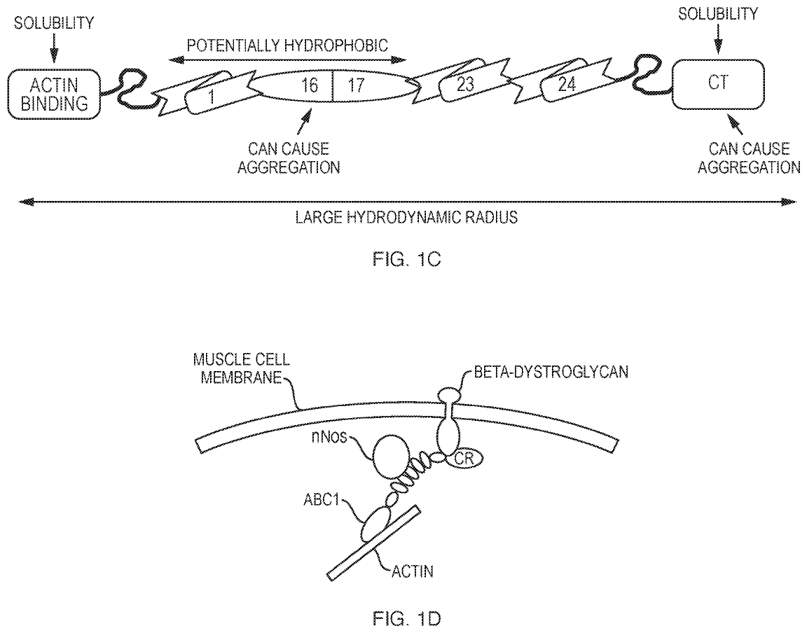

Wild type human dystrophin protein is 3685 amino acids in length and approximately 426 kDa in size (SEQ ID NO: 13, UniProt ID no. P11532, encoded by NCBI RefSeq no. NM_004006.3). The protein is organized into 31 distinct protein domains as shown in FIG. 1A, including: an N-terminal actin-binding domain 1 (ABD1) comprising 2 calponin homology (CH) domains; the central rod domain comprising a first hinge domain (H1), three spectrin-like repeat (R) domains (R1, R2, R3), a second hinge domain (H2), R domains 4-19 (R4, R5, R6, R7, R8, R9, R10, R11, R12, R13, R14, R15, R16, R17, R18, R19), a third hinge domain (H3), R domains 20-24 (R20, R21, R22, R23, R24), and a fourth hinge domain (H4); and a C-terminal cysteine-rich (CR) domain comprising two coiled-coil repeats (collectively the C-terminal domain or CTD).

Spectrin-like repeat domains R1-R19 are hypothesized to function as a collective anionic phospholipid interaction super-domain, with R16 and R17 providing an interaction site for neuronal nitric oxide synthase (nNos) protein.

The cysteine-rich (CR) domain and the C-terminal domain (CTD) are thought to provide binding sites for syntrophin and dystrobrevin, as well as other proteins that may play a role in the dystrophin-associated protein complex.

In the rhMD proteins described herein, the ABD1 domain may comprise or consist of or consist essentially of the amino acid sequence set forth in SEQ ID NO: 14. In some embodiments, the ABD1 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 14.

In the rhMD proteins described herein, the CH domain 1 may comprise or consist of or consist essentially of the amino acid sequence set forth in SEQ ID NO: 15. In some embodiments, the CH domain 1 shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 15.

In the rhMD proteins described herein, the CH domain 2 may comprise or consist of or consist essentially of the amino acid sequence set forth in SEQ ID NO: 16. In some embodiments, the CH domain 2 shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 16.

In the rhMD proteins described herein, the H1 domain may comprise or consist of or consist essentially of the amino acid sequence set forth in SEQ ID NO: 17. In some embodiments, the H1 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 17.

In the rhMD proteins described herein, the H2 domain may comprise or consist of or consist essentially of the amino acid sequence set forth in SEQ ID NO: 18. In some embodiments, the H2 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 18.

In the rhMD proteins described herein, the H3 domain may comprise or consist of or consist essentially of the amino acid sequence set forth in SEQ ID NO: 19. In some embodiments, the H3 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 19.

In the rhMD proteins described herein, the H4 domain may comprise or consist of or consist essentially of the amino acid sequence set forth in SEQ ID NO: 20. In some embodiments, the H4 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 20.

In the rhMD proteins described herein, the R1 domain may comprise or consist of or consist essentially of the amino acid sequence set forth in SEQ ID NO: 21. In some embodiments, the R1 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 21.

In the rhMD proteins described herein, the R2 domain may comprise or consist of or consist essentially of the amino acid sequence set forth in SEQ ID NO: 22. In some embodiments, the R2 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 22.

In the rhMD proteins described herein, the R3 domain may comprise or consist of or consist essentially of the amino acid sequence set forth in SEQ ID NO: 23. In some embodiments, the R3 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 23.

In the rhMD proteins described herein, the R4 domain may comprise or consist of or consist essentially of the amino acid sequence set forth in SEQ ID NO: 24. In some embodiments, the R4 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 24.

In the rhMD proteins described herein, the R5 domain may comprise or consist of or consist essentially of the amino acid sequence set forth in SEQ ID NO: 25. In some embodiments, the R5 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 25.

In the rhMD proteins described herein, the R6 domain may comprise or consist of or consist essentially of the amino acid sequence set forth in SEQ ID NO: 26. In some embodiments, the R6 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 26.

In the rhMD proteins described herein, the R7 domain may comprise or consist of or consist essentially of the amino acid sequence set forth in SEQ ID NO: 27. In some embodiments, the R7 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 27.

In the rhMD proteins described herein, the R8 domain may comprise or consist of or consist essentially of the amino acid sequence set forth in SEQ ID NO: 28. In some embodiments, the R8 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 28.

In the rhMD proteins described herein, the R9 domain may comprise or consist of or consist essentially of the amino acid sequence set forth in SEQ ID NO: 29. In some embodiments, the R9 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 29.

In the rhMD proteins described herein, the R10 domain may comprise or consist of or consist essentially of the amino acid sequence set forth in SEQ ID NO: 30. In some embodiments, the R10 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 30.

In the rhMD proteins described herein, the R11 domain may comprise or consist of or consist essentially of the amino acid sequence set forth in SEQ ID NO: 31. In some embodiments, the R11 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 31.

In the rhMD proteins described herein, the R12 domain may comprise or consist of or consist essentially of the amino acid sequence set forth in SEQ ID NO: 32. In some embodiments, the R12 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 32.

In the rhMD proteins described herein, the R13 domain may comprise or consist of or consist essentially of the amino acid sequence set forth in SEQ ID NO: 33. In some embodiments, the R13 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 33.

In the rhMD proteins described herein, the R14 domain may comprise or consist of or consist essentially of the amino acid sequence set forth in SEQ ID NO: 34. In some embodiments, the R14 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 34.

In the rhMD proteins described herein, the R15 domain may comprise or consist of or consist essentially of the amino acid sequence set forth in SEQ ID NO: 35. In some embodiments, the R15 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 35.

In the rhMD proteins described herein, the R16 domain may comprise or consist of or consist essentially of the amino acid sequence set forth in SEQ ID NO: 36. In some embodiments, the R16 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 36.

In the rhMD proteins described herein, the R17 domain may comprise or consist of or consist essentially of the amino acid sequence set forth in SEQ ID NO: 37. In some embodiments, the R17 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 37.

In the rhMD proteins described herein, the R18 domain may comprise or consist of or consist essentially of the amino acid sequence set forth in SEQ ID NO: 38. In some embodiments, the R18 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 38.

In the rhMD proteins described herein, the R19 domain may comprise or consist of or consist essentially of the amino acid sequence set forth in SEQ ID NO: 39. In some embodiments, the R19 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 39.

In the rhMD proteins described herein, the R20 domain may comprise or consist of or consist essentially of the amino acid sequence set forth in SEQ ID NO: 40. In some embodiments, the R20 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 40.

In the rhMD proteins described herein, the R21 domain may comprise or consist of or consist essentially of the amino acid sequence set forth in SEQ ID NO: 41. In some embodiments, the R21 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 41.

In the rhMD proteins described herein, the R22 domain may comprise or consist of or consist essentially of the amino acid sequence set forth in SEQ ID NO: 42. In some embodiments, the R22 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 42.

In the rhMD proteins described herein, the R23 domain may comprise or consist of or consist essentially of the amino acid sequence set forth in SEQ ID NO: 43. In some embodiments, the R23 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 43.

In the rhMD proteins described herein, the R24 domain may comprise or consist of or consist essentially of the amino acid sequence set forth in SEQ ID NO: 44. In some embodiments, the R24 domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 44.

In the rhMD proteins described herein, the CR domain may comprise or consist of or consist essentially of the amino acid sequence set forth in SEQ ID NO: 45. In some embodiments, the CR domain shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 45.

In the rhMD proteins described herein, the CTD may comprise or consist of or consist essentially of the amino acid sequence set forth in SEQ ID NO: 46. In some embodiments, the CTD shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 46.

In some embodiments, the rhMD protein comprises, in order from the rhMD's N-terminus to the rhMD protein's C-terminus, the following elements: an NTD; an H1 domain; an R1 domain; an R16 domain; an R17 domain; an R23 domain; an R24 domain; an H4 domain; and a beta-dystroglycan binding domain. In some embodiments, the rhMD protein comprises, in order from the rhMD's N-terminus to the rhMD protein's C-terminus, the following elements: an NTD or a fragment thereof; an H1 domain or a fragment thereof; an R1 domain or a fragment thereof; an R16 domain or a fragment thereof; an R17 domain or a fragment thereof; an R23 domain or a fragment thereof; an R24 domain or a fragment thereof; an H4 domain or a fragment thereof; and a beta-dystroglycan binding domain or a fragment thereof.

Various features of the dystrophin protein have complicated efforts to express and purify a recombinant, soluble, non-denatured form of the protein. For instance, the potentially hydrophobic central rod domain may contribute to aggregation of recombinantly expressed proteins. Particular spectrin-like repeat domains, like R16 and R17, may contribute more to hydrophobicity/aggregative characteristics than other spectrin-like repeat domains. The cysteine-rich C-terminal domain is additionally thought to contribute to aggregation. Further, the size of the protein itself and its large hydrodynamic radius have stymied efforts to express the protein in vitro. These complications have hampered development and characterization of recombinant forms of the dystrophin protein that are small enough to be translatable into rAAV-mediated or other gene therapy vectors while retaining the necessary functional and binding domains of the dystrophin protein. Even engineered truncated forms of the dystrophin protein (microdystrophins) have proven highly difficult to express in vitro in soluble, non-denatured form.

rhMD Protein 1

The rhMD protein 1 described herein is a 147 kDa truncated version of the wild type human dystrophin protein. rhMD protein 1 comprises a core structure of the N-terminal domain (NTD, which comprises ABD1 having CH domains), a central rod domain with five spectrin-like repeat domains (R1, R16, R17, R23 and R24) between two hinge domains (H1 and H4), followed by a cysteine-rich (CR) domain (FIG. 1B). In some embodiments, the rhMD protein 1 comprises a core structure of the N-terminal domain (NTD, which comprises ABD1 having CH domains) or a fragment thereof, a central rod domain with five spectrin-like repeat domains (R1 or a fragment thereof, R16 or a fragment thereof, R17 or a fragment thereof, R23 or a fragment thereof and R24 or a fragment thereof) between two hinge domains (H1 domain or a fragment thereof and H4 domain or a fragment thereof), followed by a cysteine-rich (CR) domain or a fragment thereof (FIG. 1B).

The core structure of rhMD protein 1 disclosed herein retains the structural elements that contribute to hydrophobicity, protein aggregation and solubility, and anchoring to the cell membrane (see FIGS. 1C and 1D), all of which have complicated prior attempts to express and purify a soluble, non-denatured form of the protein.

Examples of rhMD protein 1 are described, e.g., in US Patent Application Publication Nos. 2018/0148488 A1, US 2020/0095298 A1, and US 2020/0031890 A1, the contents of which are incorporated herein by reference.

In some embodiments, the rhMD protein 1 has an amino acid sequence set forth in SEQ ID NO: 1. In some embodiments, the rhMD protein 1 has an amino acid sequence that shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 1.

rhMD Protein 2

The rhMD protein 2 described herein is a truncated version of the wild type human dystrophin protein. rhMD protein 2 comprises a core structure of the N-terminal domain (NTD), a central rod domain with five spectrin-like repeat domains (R1, R2, R22, R23, and R24) between two hinge domains (H1 and H4), with hinge domain H3 between R2 and R22, followed by a CR domain (FIG. 1B).

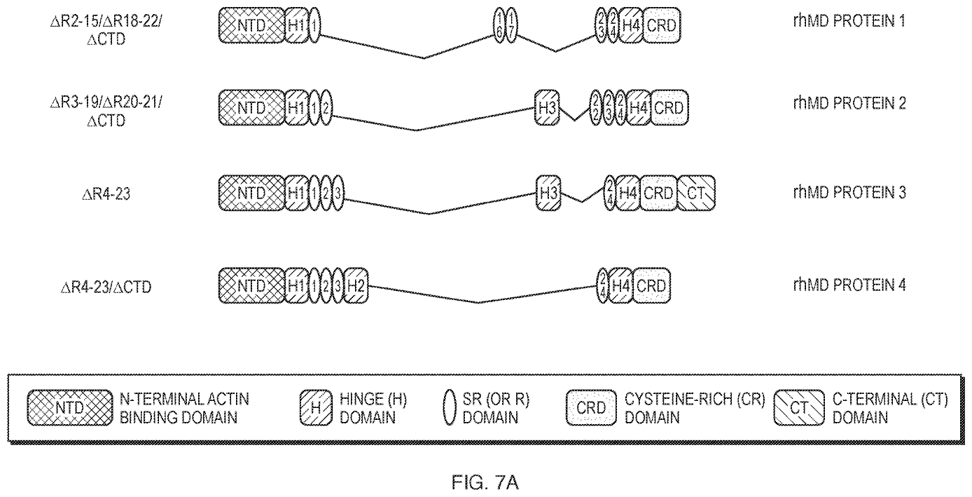

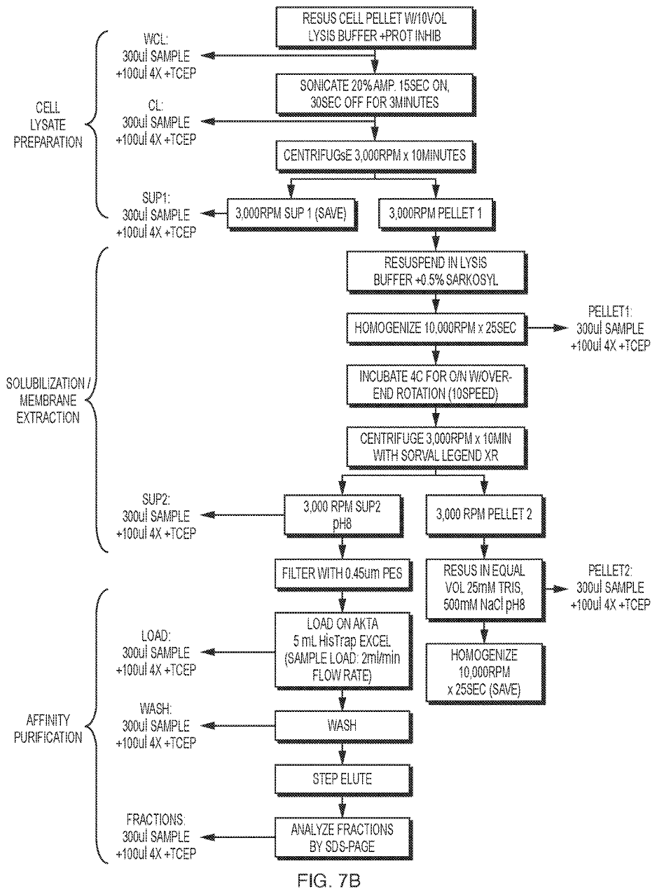

In some embodiments, the rhMD protein 2 described herein is a truncated version of the wild type human dystrophin protein. rhMD protein 2 comprises a core structure of the N-terminal domain (NTD) or a fragment thereof, a central rod domain with five spectrin-like repeat domains (R1 or a fragment thereof, R2 or a fragment thereof, R22 or a fragment thereof, R23 or a fragment thereof, and R24 or a fragment thereof) between two hinge domains (H1 domain or a fragment thereof and H4 domain or a fragment thereof), with hinge domain H3 or a fragment thereof between R2 domain or a fragment thereof and R22 domain or a fragment thereof, followed by a CR domain or a fragment thereof (FIG. 7A).

Examples of rhMD protein 2 are described, e.g., in WIPO Patent Application Publication No. WO/2020/261178 A1, the contents of which are herein incorporated by reference.

In some embodiments, the rhMD protein 2 has an amino acid sequence set forth in SEQ ID NO: 2. In some embodiments, the rhMD protein 2 has an amino acid sequence that shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 2.

rhMD Protein 3

The rhMD protein 3 described herein is a truncated version of the wild-type human dystrophin protein. rhMD protein 3 comprises a core structure of the N-terminal domain (NTD), a central rod domain with four spectrin-like repeat domains (R1, R2, R3,) between two hinge domains (H1 and H3), with R24 between hinge domain H3 and hinge domain 4, followed by a CR domain and extended C terminal domain (FIG. 1B).

In some embodiments, the rhMD protein 3 described herein is a truncated version of the wild-type human dystrophin protein. rhMD protein 3 comprises a core structure of the N-terminal domain (NTD) or a fragment thereof, a central rod domain with four spectrin-like repeat domains (R1 or a fragment thereof, R2 or a fragment thereof, R3 or a fragment thereof,) between two hinge domains (H1 domain or a fragment thereof and H3 domain or a fragment thereof), with R24 or a fragment thereof between hinge domain H3 or a fragment thereof and hinge domain 4 or a fragment thereof, followed by a CR domain or a fragment thereof and extended C terminal domain or a fragment thereof (FIG. 7A).

Examples of rhMD protein 3 are described, e.g., in WIPO Patent Application Publication Nos. WO/2021108755 A1 and WO/2022/232141 A1, the contents of which are herein incorporated by reference.

In some embodiments, the rhMD protein 3 has an amino acid sequence set forth in SEQ ID NO: 3. In some embodiments, the rhMD protein 3 has an amino acid sequence that shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 3.

rhMD Protein 4

The rhMD protein 4 described herein is a truncated version of the wild type human dystrophin protein. rhMD protein 4 comprises a core structure of the N-terminal domain (NTD), a central rod domain with four spectrin-like repeat domains (R1, R2, R3, and R24) between two hinge domains (H1 and H4), with hinge domain H2 between R3 and R24, followed by a CR domain (FIG. 1B).

In some embodiments, the rhMD protein 4 described herein is a truncated version of the wild type human dystrophin protein. rhMD protein 4 comprises a core structure of the N-terminal domain (NTD) or a fragment thereof, a central rod domain with four spectrin-like repeat domains (R1 or a fragment thereof, R2 or a fragment thereof, R3 or a fragment thereof, and R24 or a fragment thereof) between two hinge domains (H1 domain or a fragment thereof and H4 domain or a fragment thereof), with hinge domain H2 or a fragment thereof between R3 or a fragment thereof and R24 or a fragment thereof, followed by a CR domain or a fragment thereof (FIG. 7A)

Examples of rhMD protein 4 are described, e.g., in US Patent Application Publication No. 2021/0393801 A1, the contents of which are herein incorporated by reference.

In some embodiments, the rhMD protein 4 has an amino acid sequence set forth in SEQ ID NO: 4. In some embodiments, the rhMD protein 4 has an amino acid sequence that shares at least 90%, at least 91%, at least 92%, at least 93%, at least 94%, at least 95%, at least 96%, at least 97%, at least 98%, at least 99%, or 100% sequence identity with the amino acid sequence set forth in SEQ ID NO: 4.

### Solubility, Detection, and Affinity Tags

Various protein tags are known in the art. Such tags may be added to recombinant polypeptide sequences to provide additional functional traits. For instance, many tags are known which can be used to detect a particular recombinant polypeptide, i.e., in a sample. Other tags are known which can be used to isolate a particular polypeptide from a sample (i.e., “affinity” or “purification” tags). Still other tags are known which can be used to improve solubility of a particular recombinant polypeptide.

To improve solubility, detectability, and or purification capability, rhMD proteins may further comprise detection, affinity, and/or solubility tags. Examples of tags suitable for use in rhMDs include, but are not limited to: small ubiquitin-like modifier “SUMO” tag (SEQ ID NO: 5); Lipoyl domain “Lipoyl” tag (SEQ ID NO: 6); maltose binding protein “MBP” tag (SEQ ID NO: 7); glutathionine S-transferase “GST” tag (SEQ ID NO: 8); polyhistidine tag (such as 6×His tag, SEQ ID NO: 9); Twin-Strep-Tag® “TwinStrep” (SEQ ID NO: 10); and/or green fluorescence protein “GFP” tag (SEQ ID NO: 11); including variants of any of the foregoing. Persons skilled in the art will appreciate numerous other tags suitable for use in the compositions and methods described herein.

The rhMD proteins and uses thereof that are described in this application may comprise or utilize a first peptide binding tag linked to the N- or the C-terminus to generate a rhMD first binding partner protein. In some embodiments, the rhMD first peptide binding tag (or first binding partner protein comprising the same) can bind (e.g., irreversibly or reversibly) to a second peptide binding partner (or second binding partner protein comprising the same). The second peptide binding partner (or second binding partner protein comprising the same) may be labeled with a detectable moiety. The first peptide binding tag and the second peptide binding partner can be or be derived from partnered systems known in the art, including but not limited to, BioRad SpyCatcher and SpyTag products, Kerafast Biotech SpyTag/SpyCatcher Protein Coupling Reagents or Addgene SpyTag-β-Lactamase-SpyCatcher proteins.

In some embodiments, the first peptide binding tag may comprise an amino acid sequence selected from SEQ ID NO: 47, SEQ ID NO: 48, and SEQ ID NO: 49. In some embodiments, the second peptide binding partner may comprise an amino acid sequence selected from SEQ ID NO: 50, SEQ ID NO: 51, and SEQ ID NO: 52. In certain embodiments, the first peptide binding tag or a fragment thereof can be linked to the N- or to the C-terminus of a rhMD protein comprising an amino acid sequence according to SEQ ID NOs: 1-4. In certain embodiments, the rhMD protein linked to a first peptide binding tag may comprise an amino acid sequence according to SEQ ID NO: 53 (rhMD protein 1-HIS-SpyTag) or SEQ ID NO: 54 (rhMD protein 1-SpyTag-HIS).

These tags and other tags can be present at the N-terminus of the rhMD and/or at the C-terminus of the rhMD. These tags can be present in the rhMD alone or in combination with other tags or in combination with each other. Persons skilled in the art will appreciate that fusion of one or more of these tags may involve direct fusion of the amino acid sequence of the one or more tags directly together or directly to a rhMD, or alternatively may include appropriate spacers or linkers (such as additional amino acids or other chemical linkers) between the one or more tags and/or between the tags and the rhMD.

In some cases, it is desirable to be able to remove one or more tags fused to recombinant proteins. Various protein cleavage or enzyme degradable sites are known in the art. Such cleavage sites can be introduced along with tags as described herein to facilitate removal of the tags post-expression and/or post-purification. An exemplary cleavage site useful in the rhMDs described herein is the tobacco etch virus “TEV” protease cleavage site linker (SEQ ID NO: 12). Persons skilled in the art will recognize various other cleavage sites that can be substituted in the tagged rhMD proteins described herein.

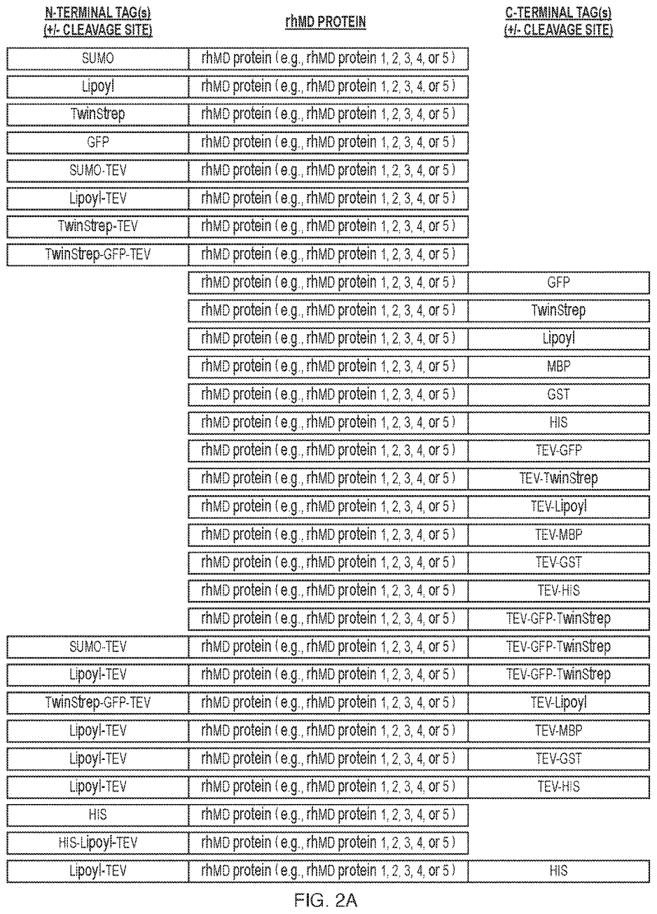

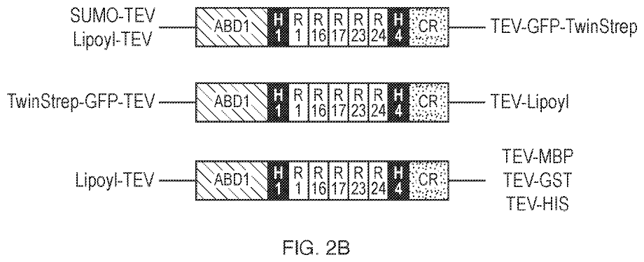

FIG. 2A shows various configurations of solubility, detection, and affinity tags that can be added to rhMD proteins according to embodiments of the methods and compositions described herein.

FIG. 2B shows configurations of certain tagged versions of rhMD protein 1 with the N-terminus of a rhMD be tagged with a SUMO tag (“SUMO”) and/or a TEV cleavage site linker (“TEV”) or a Lipoyl tag (“Lipoyl”) and/or a TEV cleavage site; or a TwinStrep tag (“TwinStrep”) and/or a GFP tag (“GFP”) and/or a TEV cleavage site linker. Similarly, the C-terminus may be tagged, e.g., with a TEV cleavage site linker and/or a GFP tag and/or a TwinStrep tag; a TEV cleavage site linker and/or a Lipoyl tag; or a TEV cleavage site linker and/or a MBP tag, a TEV cleavage site linker and/or a GST tag, or a TEV cleavage site linker and/or a HIS tag.

Methods of Producing Soluble, Non-Denatured rhMDs

Provided herein are methods of producing recombinant human microdystrophin (rhMD) protein. In some embodiments, the methods enable reliable production of rhMD protein in solution. In some embodiments, the methods enable reliable production of non-denatured rhMD protein.

The rhMD protein produced according to the methods described herein can be any recombinant microdystrophin protein. In some embodiments, the rhMD protein comprises an N-terminal domain (NTD) comprising an actin-binding domain (ABD1) or a fragment thereof.

In some embodiments, the rhMD protein comprises various spectrin-like repeat (R) domains or their respective fragments, but typically fewer than all 24 of the R domains from wild-type dystrophin protein. For instance, the rhMD protein may have from one to five R domains. In some embodiments, the rhMD protein comprises between 1 and 5 R domains selected from R1 domain or a fragment thereof, R2 domain or a fragment thereof, R3 domain or a fragment thereof, R16 domain or a fragment thereof, R17 domain or a fragment thereof, R22 domain or a fragment thereof, R23 domain or a fragment thereof, and R24 domain or a fragment thereof.

In some embodiments, the rhMD protein comprises a cysteine-rich C-terminal domain (CTD) or a fragment thereof. The CTD may comprise beta-dystroglycan binding domain or a fragment thereof and/or a syntrophin binding domain or a fragment thereof.

In some embodiments, the rhMD protein comprises an N-terminal actin binding domain (NTD) or a fragment thereof; at least one hinge domain or hinge-like domain (H) or at least a portion of a hinge domain selected from H1 domain or a fragment thereof, H2 domain or a fragment thereof, H3 domain or a fragment thereof, H4 domain or a fragment thereof and a hinge-like domain; one to five spectrin-like repeat (R) domains selected from R1 domain or a fragment thereof, R2 domain or a fragment thereof, R3 domain or a fragment thereof, R16 domain or a fragment thereof, R17 domain or a fragment thereof, R22 domain or a fragment thereof, R23 domain or a fragment thereof, and R24 domain or a fragment thereof; and a beta-dystroglycan binding domain or a fragment thereof. For instance, the rhMD protein may comprise five R domains selected from R1 domain or a fragment thereof, R16 domain or a fragment thereof, R17 domain or a fragment thereof, R23 domain or a fragment thereof, and R24 domain or a fragment thereof, and a beta-dystroglycan binding domain or a fragment thereof.

In some embodiments, the rhMD protein comprises an NTD coupled to a H1 domain coupled to a R1 domain coupled to a R16 domain coupled to a R17 domain coupled to a R23 domain coupled to a R24 domain coupled to a H4 domain coupled to a beta-dystroglycan binding domain. In some embodiments, the rhMD protein comprises an NTD or a fragment thereof coupled to a H1 domain or a fragment thereof coupled to a R1 domain or a fragment thereof coupled to a R16 domain or a fragment thereof coupled to a R17 domain or a fragment thereof coupled to a R23 domain or a fragment thereof coupled to a R24 domain or a fragment thereof coupled to a H4 domain or a fragment thereof coupled to a beta-dystroglycan binding domain or a fragment thereof.

Exemplary rhMD proteins include rhMD protein 1 having an amino acid sequence set forth in SEQ ID NO: 1, rhMD protein 2 having an amino acid sequence set forth in SEQ ID NO: 2, rhMD protein 3 having an amino acid sequence set forth in SEQ ID NO: 3, and rhMD protein 4 having an amino acid sequence set forth in SEQ ID NO: 4.

The rhMD proteins produced according to the methods described herein may further comprise one or more non-dystrophin-derived functional domains. Such non-dystrophin derived functional domains may include detection tags, purification tags, and/or solubility tags which, generally, are peptide functional domains, the amino acid sequences of which can be fused to a protein sequence, for example at the N-terminus and/or at the C-terminus.

Examples of solubility tags include the small ubiquitin-like modifier tag (SUMO tag) and the Lipoyl domain tag (Lipoyl tag). In some embodiments, the rhMD proteins described herein comprise a SUMO tag and/or a Lipoyl tag at the N-terminus, at the C-terminus, or at the N- and the C-terminus.

Examples of detection tags include maltose binding protein tag (MBP tag), glutathione S-transferase tag (GST tag), histidine tag (HIS tag), green fluorescence protein-based tags (GFP), and Streptavidin-based tags (Strep tags, such as Twin-Strep-Tag® or “TwinStrep”). In some embodiments, rhMD proteins described herein comprise at least one MBP tag, at least one GST tag, at least one HIS tag, at least one GFP, and/or at least one Strep tag (e.g., TwinStrep tag) at the N-terminus, at the C-terminus, or at the N- and C-terminus.

Non-dystrophin-derived functional domains such as solubility, detection, and affinity tags, can be fused to the rhMD proteins of the present disclosure with or without cleavable domains for removal of the fused domains from the rhMD protein after purification. An example of a suitable cleavable domain is the tobacco etch virus (TEV) protease cleavage site linker, which can be introduced between peptide domains, and which can be exploited to cleave the expressed protein at the TEV site.

Generally, rhMD proteins expressed in host cell cultures will be bound to, embedded in, or strongly associated with the cell membranes of the host cells, necessitating efficient extraction or release from the membranes. For the rhMD proteins to be useful in downstream assays or analysis, the extraction should result in non-denatured rhMD protein.

The methods provided herein for producing these rhMD proteins generally entail lysis of rhMD-protein-expressing cells, cell fractionation by centrifugation, then release of the rhMD protein from the membrane fraction. The methods may further entail subsequent purification of rhMD protein. The methods may further comprise steps to remove any detection, solubility, and/or affinity tags present on the rhMD protein. The disclosed process advantageously produces soluble and non-denatured rhMD protein.

In some embodiments, the methods comprise contacting rhMD-protein-expressing cells with a lysis buffer, preparing a cell membrane fraction from the cells, then extracting the rhMD protein from the cell membrane fraction using an anionic detergent.

In one aspect, the present disclosure provides a method of producing a non-denatured recombinant human microdystrophin (rhMD) protein in solution from a host cell culture expressing the same. The method may comprise contacting the host cell culture with a lysis buffer to generate a cell lysate, then centrifuging the cell lysate to generate at least one cell lysate fraction comprising plasma membranes of the host cells, e.g., a cell membrane fraction, and contacting the cell membrane fraction with a membrane extraction buffer comprising an anionic detergent to induce release of the rhMD protein.

Host cells expressing the rhMD proteins may be prepared, e.g., by transient transfection of a plasmid DNA encoding the rhMD protein in a cell suitable for expression of the same. Alternatively, the host cells expressing the rhMD protein may stably express a transgene encoding the rhMD protein(s). Methods of producing host cells transiently transfected with a plasmid DNA and/or stably expressing a transgene of interest will be known by persons skilled in the art.

Host cells suitable for expressing the rhMD proteins include HEK293 cells (such as Expi293F™ cells from ThermoFischer Scientific), HEK293T cells, BHK cells, CHO cells, COS cells, and/or HeLa cells. Once transfected or modified to stably express the rhMD, the host cell culture can be maintained for a duration of time and under conditions appropriate for protein expression. Culture conditions which can be optimized according to routine methodology include selection and supplementation of growth media, culture format (suspension versus adherent culture, for example), incubation time, incubation temperature, pH, dissolved oxygen levels, and the like.

The host cell culture can be maintained under conditions for expressing the rhMD protein, e.g., for at least about 6 hours, at least about 12 hours, at least about 18 hours, at least about 24 hours, at least about 30 hours, at least about 36 hours, at least about 42 hours, at least about 48 hours, at least about 54 hours, at least about 60 hours, at least about 66 hours, at least about 72 hours, at least about 78 hours, at least about 84 hours, at least about 90 hours, or at least about 96 hours.

The percentage of live cells in a host cell culture is expected to decrease over time, which may reduce the ability to recover expressed protein over time. In some embodiments, the host cell culture can be maintained for an amount of time until the percentage of live cells of the host cell culture post-transfection with the nucleic acid encoding the rhMD protein is at least about 95%, at least about 90%, at least about 80%, at least about 70%, at least about 60%, or at least about 50%. The percentage of live cells present in a cell culture can be expressed as viability of the cell culture. Viability may be evaluated, e.g., by microscopy-based assays, colorimetry-based assays, or flow-cytometry-based assays, or combinations thereof.

In transiently transfected host cell cultures, the percentage of transfected cells may decrease over time, which may reduce the ability to recover expressed protein over time. In some embodiments, the host cell culture can be maintained for an amount of time until the percentage of transfected cells in the host cell culture post-transfection with the nucleic acid encoding the rhMD protein is at least about 95%, at least about 90%, at least about 80%, at least about 70%, at least about 60%, or at least about 50%. The percentage of transfected cells may be evaluated, e.g., by microscopy-based assays, colorimetry-based assays, or flow-cytometry-based assays, or combinations thereof.

Following culturing of the host cells, the cells can be contacted with a lysis buffer to lyse the host cell culture. The lysis buffer may comprise a detergent. The detergent may be, e.g., a non-ionic detergent, a cationic detergent, an anionic detergent or a zwitterionic detergent. Numerous detergents suitable for lysing cultured host cells are known. In some embodiments, the detergent is selected from Triton X-100, Triton X-114, Triton-200, sodium dodecyl sulfate, NP-40, Tween 20, Tween 80 and CHAPS.

The cells can be contacted with the lysis buffer for an amount of time sufficient to generate a cell lysate.

The cell lysate can be centrifuged to generate at least one cell lysate fraction comprising plasma membranes of the cell culture (cell membrane fractionation). Centrifugation can be performed, e.g., at about 1000×g to about 12000×g, for example, at about 1000×g, about 1100×g, about 1200×g, about 1300×g, about 1400×g, about 1500×g, about 2000×g, about 2500×g, about 3000×g, about 3500×g, about 4000×g, about 4500×g, about 5000×g, about 5500×g, about 6000×g, about 6500×g, about 7000×g, about 7500×g, about 8000×g, about 8500×g, about 9000×g, about 9500×g, about 10000×g, about 11000×g, or about 12000×g. The centrifugation can be performed, e.g., for about 5 minutes to about 1 hour, for example about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes.

The cell membrane fraction can be contacted with a membrane extraction buffer to induce release of the rhMD protein. In some embodiments, the membrane extraction buffer comprises an anionic detergent to induce release of the rhMD protein. The membrane extraction buffer may comprise, e.g., about 0.1% w/v to about 1.0% w/v of the anionic detergent. For example, the membrane extraction buffer may comprise about 0.1% w/v, about 0.2% w/v, about 0.3% w/v, about 0.4% w/v, about 0.5% w/v, about 0.6% w/v, about 0.7% w/v, about 0.8% w/v, about 0.9% w/v, or about 1.0% w/v of the anionic detergent. Examples of anionic detergents are known and include sodium dodecyl sulfate (SDS), sodium deoxycholate (SD), sulfonic acid salt, alcohol sulfate, alkylbenzene sulfonate, phosphoric acid ester, carboxylic acid salt, ammonium lauryl sulfate, sodium laureth sulfate, sodium lauryl sarcosinate (sarkosyl), potassium lauryl sarcosinate sodium myreth sulfate, sodium pareth sulfate, sodium stearate, sodium lauryl sulfate, a olefin sulfonate, and ammonium laureth sulfate. Contacting the cell membrane fraction with the membrane extraction buffer may be carried out, e.g., for a few minutes to about half an hour. In some embodiments, the cell membrane fraction is contacted with the membrane extraction buffer for about 5 minutes, about 10 minutes, about 15 minutes, about 20 minutes, about 25 minutes, or about 30 minutes to release the rhMD protein. In some embodiments, the cell membrane fraction is contacted with the membrane extraction buffer for about 16 minutes to about 24 minutes to release the rhMD protein.

The methods of producing rhMD protein described herein may further comprise steps to purify the released rhMD protein. Exemplary methods for purifying exogenously expressed recombinant protein are known. In some embodiments, affinity chromatography may be used to purify the rhMD protein. For instance, an affinity chromatography resin may be used to capture the rhMD protein from a solution comprising the released rhMD protein. The affinity may be, for example, immunoaffinity-mediated, such as with an antibody or antigen-binding fragment specific to an antigen present in the rhMD protein. Alternatively, the chromatography resin may have an affinity to a particular affinity tag present on the rhMD protein. Examples of chromatography resins that can be used to purify rhMD proteins include reduced-glutathione chromatography resin (as in the case of GST-tagged rhMD protein), Ni-NTA chromatography resin (as in the case of a HIS-tagged rhMD protein), or an amylose chromatography resin (as in the case of MBP-tagged rhMD protein).

Certain embodiments of the methods for producing rhMD proteins described herein comprise culturing Expi293F cells transiently transfected with a plasmid DNA encoding the rhMD protein and harvesting the cells, e.g., by centrifugation to generate a cell pellet. Post-harvest of the Expi293 cells, the cell pellet may be lysed in a lysis buffer. The cell pellet may additionally or alternatively be sonicated to disrupt cell membranes. Next, lysed cells may be centrifuged to obtain a post-lysis cell pellet and supernatant. The post-lysis cell pellet may be resuspended and homogenized in a membrane extraction buffer. An example of a membrane extraction buffer is a buffer comprising 0.5% sarkosyl. The post-lysis cell pellet may be incubated, e.g., overnight at 4° C. in the membrane extraction buffer for extracting the rhMD protein. As an example, centrifugation may be used to generate a post-solubilization supernatant and an insoluble pellet. The supernatant post-solubilization may be further filtered and loaded onto a chromatography column, e.g., a Ni-NTA or HisTrap column, using step elution at pH 8. The loaded chromatography column may be washed and eluted to obtain elution fractions which can be analyzed for presence of the purified rhMD protein. Western blotting using an antibody specific to the rhMD or other suitable techniques may be used to detect the rhMD protein at any step of the process.

Uses of Soluble, Non-Denatured rhMD Proteins

The present disclosure provides methods for reliably producing rhMD proteins. The rhMD proteins produced according to the presently disclosed methods may be soluble or solubilized. The rhMD proteins produced according to the presently disclosed methods may be non-denatured. Accordingly, the rhMD proteins produced according to the presently disclosed methods can be used to characterize the rhMD proteins and/or to carry out in vitro analytical assays, for instance, where there is an interest in detecting presence of microdystrophin peptides and microdystrophin-specific antibodies in samples. Various uses of the rhMD proteins produced according to the presently disclosed methods are further described below.

In some embodiments, the rhMD proteins produced according to the present disclosure are useful in analytical methods for detecting microdystrophin-specific antibodies in a sample. For example, the rhMD proteins produced by the methods described herein can be used to detect microdystrophin-specific anti-drug-antibodies (ADAs) in a sample.

ADAs may be raised by subject immune systems after receiving a particular drug, limiting effectiveness of the drug or limiting the patient's tolerability of further doses of the drug. For viral vector-mediated gene therapies in particular, immunogenicity of the genetic material and/or the therapeutic gene product can give rise to humoral and cellular immune responses, including development of ADAs, in patients administered the gene therapy. This patient immune response can lead to loss of efficacy of the gene therapy over time and may impede the possibility of re-dosing subjects. The ability to evaluate immune responses and ADA formation in pre-clinical and clinical trials of rhMDs is critical to show and improve therapeutic efficacy as well as manage patient immune responses to the therapies. However, the difficulties faced in reliably producing soluble, non-denatured rhMD protein have thwarted development of ADA detection assays for rhMD therapeutics.

Accordingly, the rhMDs produced according to the present methods enable detection of ADAs raised in subjects administered a therapeutic microdystrophin gene or protein. The subjects may be human subjects or animals receiving rhMD-based gene therapies.

Analytical methods for detecting ADAs may be, e.g., based on colorimetric methods, enzyme immunoassays (EIA), radioimmunoassays (RIA), fluoroimmunoassays (FIA), chemiluminescent immunoassays (CLIA), or counting immunoassays (CIA).

Generally, analytical methods for detecting microdystrophin-specific antibodies in a sample comprise immobilizing a rhMD protein or a fragment thereof to a solid surface, contacting the immobilized rhMD protein with the sample to bind the immobilized rhMD protein to the microdystrophin-specific antibody (if present in the sample), forming a bipartite complex of the rhMD protein and the microdystrophin-specific antibody, then contacting the bipartite complex with a detection reagent that binds specifically to the microdystrophin-specific antibody and not the rhMD protein, and forming a tripartite complex of the detection reagent, the rhMD protein, and the dystrophin-specific antibody. In some embodiments, the detection reagent comprises a detectable moiety that generates a detectable signal, enabling detection of the signal to determine the presence of the microdystrophin-specific antibodies in the sample.

Immobilizing the rhMD protein or fragment thereof may involve coupling the rhMD protein or fragment to a solid surface. For example, the rhMD protein or fragment may be immobilized via interaction with an anti-microdystrophin antibody which is crosslinked to the solid surface. Alternatively, the rhMD protein or fragment may be covalently crosslinked to the solid surface.

Exemplary solid surfaces for immobilizing a rhMD protein or fragment include polyethylene, polyacrylamide, polystyrene, agarose, glass, or silicone rubber substrates.

Analytical methods for detecting microdystrophin-specific antibodies in a sample comprise immobilizing a rhMD protein or a fragment thereof may also comprise additional steps to reduce non-specific interactions. For example, the methods may comprise contacting the immobilized rhMD protein with a coating buffer or a blocker before proceeding with applying the detection reagent. The methods may also comprise, e.g., a step of contacting the bipartite complex with a wash buffer before proceeding with applying the detection reagent. Once the detection reagent is added, an additional wash step using a wash buffer can be performed before proceeding to detect a signal from the detection reagent.

Detection reagents useful in the present methods may comprise, e.g., a detectable moiety selected from an enzyme, a fluorophore, or a radioisotope. The detectable signal from the detectably moiety may be, e.g., a colorimetric signal, a fluorescent signal, or radiation. In some embodiments, the detectable moiety is an enzyme, and detecting the signal comprises contacting the tripartite complex with a substrate for generating the detectable signal. For example, the enzyme may be, e.g., a horse radish peroxidase, an alkaline phosphatase, a galactosidase, an acetylcholinesterase, or a catalase.

In some embodiments, the methods for detecting a rhMD-specific antibody in a sample further comprises a step of contacting the rhMD protein—wherein the rhMD protein comprises a first peptide binding tag or a fragment thereof (e.g., a SpyTag protein or a protein comprising an amino acid sequence of any one of SEQ ID NOs: 47-49)—with a second peptide binding partner or a fragment thereof (e.g., SpyCatcher protein or a protein comprising an amino acid sequence of any one of SEQ ID NOs: 50-52) labelled with Ruthenium (Ru), before contacting the bipartite complex with a detection reagent.

Examples of fluorophore-based detectable moieties include fluorescein isothiocyanate (FITC), phycoerythrin (PE) or a conjugate thereof, Alexa Fluor® dyes, rhodamine dyes, Cyanine-based dyes, orange G-dyes, sulforhodamine 101 acid chloride dye (Texas red), and the like.

Examples of radioisotope-based detectable moieties include ⁹⁰Y, ¹¹¹In, ¹⁷⁷Lu, ^{99m}Tc ¹²³I, ¹²⁵I, and ¹³¹I.

Detection reagents in the ADA-detecting methods described herein can be any detection reagent that specifically binds to a rhMD protein, such as an antibody or a fragment thereof. Examples of microdystrophin-specific antibodies include MANDYS106, Dys B, DYS1-CE, DYS2-CE, and DYS3-CE antibodies.

In another aspect, the recombinant human microdystrophin (rhMD) proteins produced by the methods described herein, or fragments thereof, can be used to prepare a standard solution for use in a method of quantitatively measuring an amount of microdystrophin present in a sample solution. Measuring an amount of a microdystrophin protein present in a sample is powerful way to evaluate potency of a candidate rhMD therapeutic. For example, clinical and preclinical subjects may be administered a gene therapy product, but the amount of microdystrophin protein expressed in cells of the subject may vary. Confirming that the therapeutic protein is expressed at levels sufficient to maintain a therapeutic effect is an important aspect of validating any candidate therapy. However, validating an analytical method for quantitatively measuring microdystrophin protein present in a sample, such as a sample from a subject administered a rhMD therapeutic, requires comparison against a standard with a known amount of rhMD protein presence. Previous inability to reliably produce soluble, non-denatured rhMD proteins have made quantitative evaluation of microdystrophin proteins present in samples collected from subject difficult. Thus, the methods provided herein can produce rhMD protein for use in methods of quantitatively measuring the amount of microdystrophin present in a sample.

In some embodiments, the method of quantitatively measuring an amount of microdystrophin present in a sample solution entails a colorimetric method, an enzyme immunoassay (EIA), a radioimmunoassay (RIA), a fluoroimmunoassay (FIA), a chemiluminescent immunoassay (CLIA), or a counting immunoassay (CIA).

The method of quantitatively measuring an amount of microdystrophin present in a sample solution may comprise, e.g., contacting the sample solution with a microdystrophin capture reagent and a microdystrophin detection reagent, and detecting a signal from the microdystrophin detection reagent in the sample solution. A signal from the microdystrophin detection reagent can be measured and then compared to measurements taken in the same manner from one or more standard solutions comprising known amounts of the rhMD protein.

For example, dilutions of the standard solution comprising the rhMD protein may be contacted with a microdystrophin capture reagent and a microdystrophin detection reagent, and a signal from the microdystrophin detection reagent in each dilution of the standard solution may be detected. In this way, a standard calibration curve can be generated using the signal from the microdystrophin detection reagent at each dilution of the standard solution. Comparing the microdystrophin detection reagent signal with the calibration curve, one can quantitatively determine the concentration of microdystrophin in the sample solution.

Dilutions of the standard solution useful to generate a calibration curve may comprise from about 0.01 ng/ml to about 50 ng/ml of rhMD protein. In some embodiments, serial dilutions of a standard solution containing a known concentration of the rhMD protein produced according to the methods described herein are generated to prepare the standard calibration curve.

In certain embodiments, the microdystrophin capture reagent binds specifically to microdystrophin in the sample solution and the rhMD protein or a fragment thereof in the standard solution. The microdystrophin capture reagent may be, e.g., an antibody or a fragment thereof that specifically binds to microdystrophin/the rhMD protein. The microdystrophin capture reagent may be immobilized, for example, to a solid surface, for use in the quantitative analytical methods described herein. For instance, the microdystrophin capture reagent may be immobilized by protein A to a solid surface. In some embodiments, the microdystrophin capture reagent is a microdystrophin-specific antibody selected from MANDYS106, Dys B, DYS1-CE, DYS2-CE, and DYS3-CE antibodies. In some embodiments, the microdystrophin capture reagent is used at a concentration between about 0.5 mg/ml to about 5 mg/ml.

In some embodiments, the microdystrophin detection reagent, on the other hand, is capable of binding specifically to microdystrophin in the sample solution and the microdystrophin protein or a fragment thereof in the standard solution but not to the microdystrophin capture reagent. That is, the microdystrophin detection reagent will not cross-react with the microdystrophin capture reagent or the solid surface to which the microdystrophin capture reagent is immobilized. The microdystrophin detection reagent can be, e.g., an antibody. In some embodiments, microdystrophin capture reagent is any one of Dys B, MANDYS106, DYS1-CE, DYS2-CE, and DYS3-CE antibody, and the microdystrophin detection reagent is an antibody that is different from the microdystrophin capture reagent. The microdystrophin detection reagent may be used at a concentration, e.g., between about 0.5 mg/ml to about 2 mg/ml.

In some embodiments, detecting a signal from the microdystrophin detection reagent may comprise contacting the detection reagent with a secondary detection reagent that specifically binds the microdystrophin detection reagent and not the microdystrophin capture reagent. The secondary detection can be, e.g., an antibody or a fragment thereof and may optionally further comprise a reporter moiety. Examples of suitable reporter moieties are known. For example, the reporter moiety may be a horse radish peroxidase, an alkaline phosphatase, a galactosidase, an acetylcholinesterase, or a catalase.

As an example, the methods described herein of quantitatively measuring microdystrophin present in a sample may comprise: contacting the sample and dilutions of a standard solution created with known amounts of rhMD protein with a microdystrophin capture reagent and a microdystrophin detection reagent in a coating or blocking buffer; detecting a signal from the microdystrophin detection reagent in the sample solution and each dilution of the standard solution (e.g., by contacting the microdystrophin detection reagent with a detection solution); and comparing the signals from the microdystrophin detection reagent in the sample solution and each dilution of the standard solution to determine the concentration of microdystrophin present in the sample.

In a particular embodiment, the rhMD protein produced according to the methods described herein can be used in an assay for determination of microdystrophin in biological samples by gel electrophoresis liquid chromatography mass spectrometry (Gel-LC/MS assay). For instance, muscle tissue or other biological sample from a subject administered an rhMD therapeutic, such as a rhMD-based gene therapy for treating DMD, may be homogenized and spiked with two or more isotopologues of the rhMD protein as calibrators. The homogenized mixture can then be separated by gel electrophoresis or other appropriate separation method. The region of the gel where microdystrophin migrates may be excised (or the rhMD may be otherwise isolated) and digested with trypsin or other proteolytic enzyme. The peptides may then be extracted and analyzed by liquid chromatography mass spectrometry (LC-MS/MS). The peptides of the two isotopologues of the rhMD protein thus act as calibrators for the unlabeled rhMD protein present in the muscle tissue or other biological sample. Examples of rhMD isotopologues include rhMD proteins prepared with nitrogen-15 (15N) and/or carbon-13 (13C) isotopes. The mass differences between rhMD produced with and without isotopically labeled nitrogen or carbon are detectable by LC-MS/MS, enabling an internal standard calibrator for quantitatively measuring the amount of rhMD protein in a sample.

The sample or sample solution may be, e.g., from one or more subjects suffering from or suspected of having a dystrophinopathy. In some embodiments, the samples or sample solutions are from subject(s) to whom a therapeutic microdystrophin gene or protein has been administered or is to be administered.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Optimization of Transfection Conditions for Expression of rhMD Proteins in a Host Cell

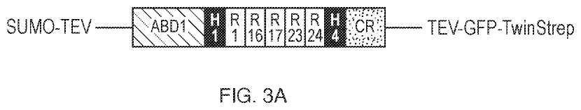

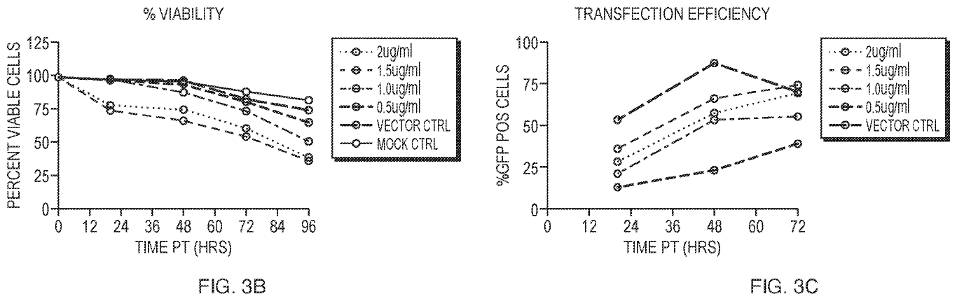

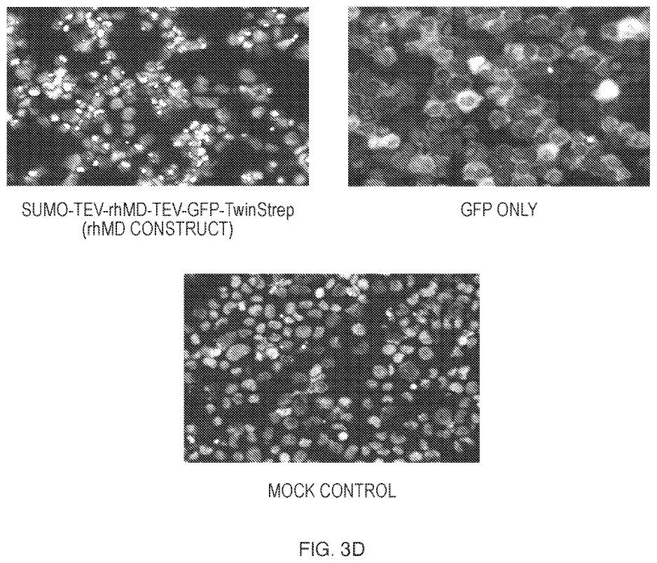

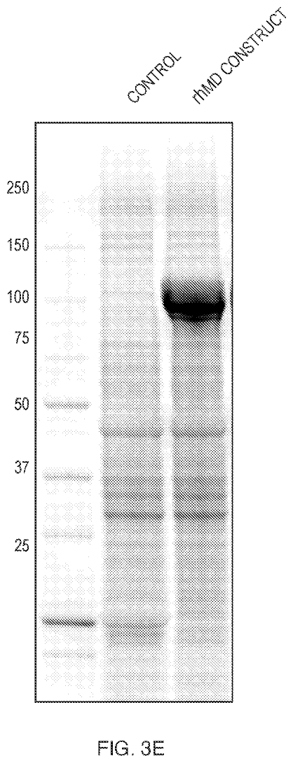

In order to standardize the conditions for optimum expression of an rhMD protein in a host cell, Expi293 cells were transfected with increasing amounts of plasmid DNA encoding rhMD protein 1 comprising a SUMO tag and TEV cleavage site linker at the N-terminus and a TEV-GFP-TwinStrep tag at the C-terminus (SUMO-TEV-rhMD-TEV-GFP-TwinStrep, FIG. 3A). Plasmid pD2529 (ATUM) was used, but other suitable expression vectors include pcDNA3.1, pcDNA3.4, pTT5, and pCI-Neo, among others. Optimal amount of DNA and time of transfection required for optimum expression of the SUMO-TEV-rhMD-TEV-GFP-TwinStrep protein were determined.

The results showed that an optimum expression of the SUMO-TEV-rhMD-TEV-GFP-TwinStrep protein was seen at about 48-72 hours post-transfection with a transfected amount of 1 to 2 μg/ml plasmid DNA. The viability of the transfected cells decreased progressively with increasing amounts of plasmid DNA and post-transfection time, with optimal viability observed for 1 to 2 μg/ml plasmid DNA, at about 48 hours post-transfection (FIGS. 3B-3C). Fluorescence microscopy and immunoblotting assays further confirmed optimum expression of the rhMD protein at 48 hours post-transfection of the plasmid DNA (FIGS. 3D-3E).

Based on these results, optimum expression of the rhMD was achieved using a transfection amount of 1 to 2 μg/ml plasmid DNA encoding the rhMD with a post-transfection time of about 48 hours. These conditions were used for subsequent studies described herein, unless otherwise noted.

### Example 2: Low Yield of rhMD Protein Following Isolation from Expressing Cell Line

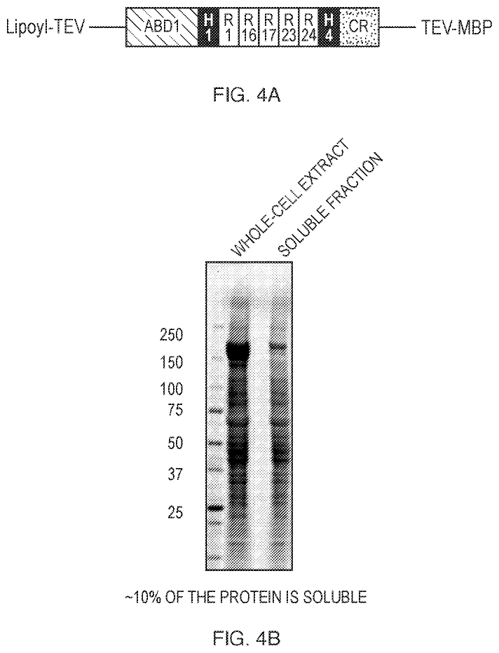

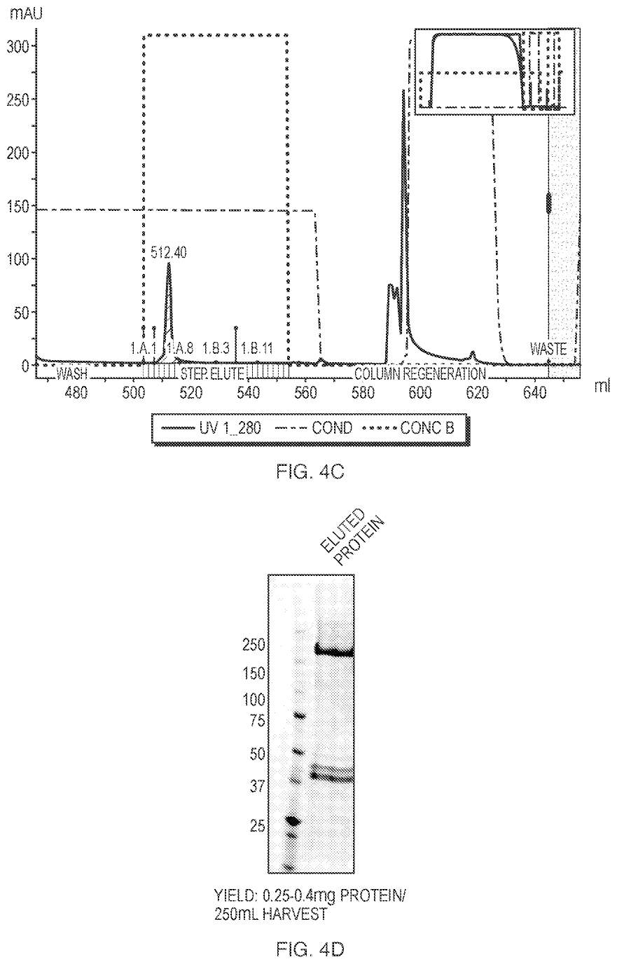

Next, protein yield was determined for rhMD protein 1 comprising a Lipoyl tag and TEV cleavage site linker at the N-terminus and a TEV-MBP tag at the C-terminus (Lipoyl-TEV-rhMD-TEV-MBP protein, FIG. 4A) expressed in the Expi293 cell line. The expressed rhMD protein was isolated by cell lysis followed by affinity purification.

The results showed that only 10% of the total Lipoyl-TEV-rhMD-TEV-MBP protein expressed in Expi293 cell line was detected in the cell lysate, indicating that a large amount of the expressed rhMD protein remained anchored to the cell membrane (see FIG. 4B). After further purification of the rhMD protein from the cell lysate using a MBPTrap purification column employing step elution method, a yield of about 0.25-0.4 mg of protein per 250 mL of cell lysate was observed (FIG. 4C-4D).

The results demonstrate the need to address the insolubility of the Lipoyl-TEV-rhMD-TEV-MBP protein by developing improved methods for extraction from the cell membrane.

### Example 3: Effect of Alkaline Extraction from Post-Lysis Cell Pellet on Solubility and Recovery of Membrane Bound Fraction of rhMD Protein

For extracting full length Dystrophin, alkaline extraction is commonly referenced as a way to separate Dystrophin from its binding partners. Sonication and alkaline extraction were tested for improving extraction of the membrane bound insoluble fraction of the Lipoyl-TEV-rhMD-TEV-MBP protein from the previous example. Expi293 cells expressing the rhMD protein as in the previous examples were lysed, and the post-lysis cell pellet was subjected to alkaline extraction with pH change (pH 11) and centrifugation at 3000 rpm. A post-extraction cell pellet and supernatant was thus prepared.

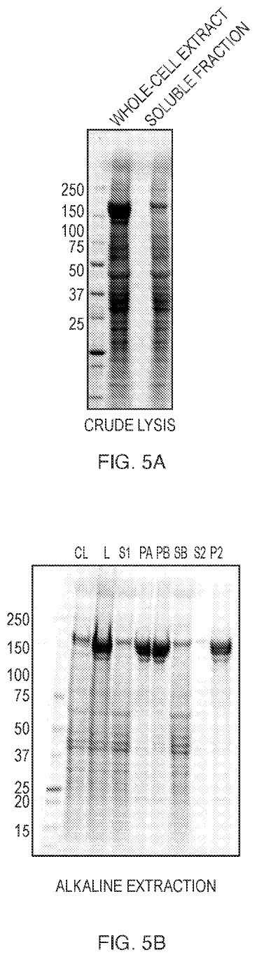

The results showed that most Lipoyl-TEV-rhMD-TEV-MBP protein remained in the cell pellet and not in the supernatant even with sonication of the post-lysis cell pellet, as well as with centrifugation of the post-lysis cell pellet after sonication with change in pH (FIG. 5B), indicating very little improvement in solubility.

Therefore, sonication and alkaline extraction from the post-lysis cell pellet was determined as not ideal for improving solubility of the rhMD protein.

### Example 4: Effect of Detergent-Extraction from Post-Lysis Cell Pellet on Solubility and Recovery of Membrane Bound Fraction of rhMD Protein

Detergent-mediated solubilization of the insoluble cell membrane bound Lipoyl-TEV-rhMD-TEV-MBP protein was next employed by incubating the post-lysis cell pellet from the Expi293 cells in non-ionic, anionic, and zwitterionic detergents.

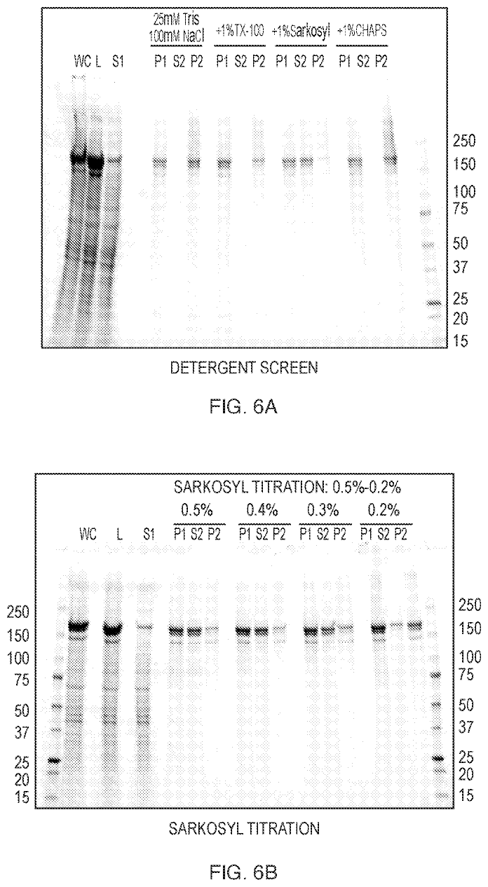

The results showed that incubation of the post-lysis cell pellet with an anionic detergent, sarkosyl, at a concentration of 1% w/v extracted a significant proportion of the insoluble Lipoyl-TEV-rhMD-TEV-MBP protein. In contrast, incubation with 1% Triton X-100 (an ionic detergent) and 1% CHAPS (a zwitterionic detergent) did not lead to any recovery of the insoluble cell membrane bound Lipoyl-TEV-rhMD-TEV-MBP protein (FIG. 6A).

Next, the effect of sarkosyl concentration was tested. The post-lysis cell pellet was incubated in sarkosyl at concentrations from 0.2% to 0.5% w/v followed by centrifugation to form a post-extraction cell pellet and supernatant. A significant proportion of the Lipoyl-TEV-rhMD-TEV-MBP protein was observed in the supernatant generated from extraction with sarkosyl concentration of 0.2-0.5% w/v, indicating improvement in solubility of the rhMD protein (FIG. 6B).

Incubation of the post-lysis cell pellet in sarkosyl at a concentration of 0.2-0.5% w/v was determined to be optimum for extraction of the insoluble cell membrane bound rhMD protein.

### Example 5: Effect of Sarkosyl Extraction on Solubility and Yield of Different rhMD Proteins

The efficacy of the disclosed method of producing rhMD protein was evaluated for improving the solubility of four different rhMD proteins: rhMD protein 1, rhMD protein 2, rhMD protein 3, and rhMD protein 4 (see FIG. 7A), each comprising a C-terminal histidine (HIS) detection tag. Plasmid DNA constructs encoding the rhMD proteins were separately transfected into Expi293 cells at 1.5 μg/ml and harvested 48 hours post-transfection. The percent viability, viable cell count, and cell pellet weight for the harvested cells for each construct are described in Table 1 below.

TABLE 1
Percentage viability, viable cell count, and cell pellet weight
for the harvested cells for rhMD protein constructs

| | | Viable Cell Count | Pellet wet |
|---|---|---|---|
| Construct | Viability (%) | (×10⁶ cells/ml) | weight (g) |
| rhMD protein 1-HIS | NA | NA | NA |
| rhMD protein 2-HIS | 90.3 | 6.64 | 13.0 |
| rhMD protein 3-HIS | 82.2 | 7.41 | 13 |
| rhMD protein 4-HIS | 93.1 | 7.28 | 16 |

Details of the experimental procedure for isolating the expressed rhMD proteins are provided in FIG. 7B. To summarize, post-harvest of the Expi293 cells expressing the rhMD protein, the cell pellet was lysed in a lysis buffer followed by sonication and centrifugation to obtain a post-lysis pellet and supernatant. The post-lysis cell pellet was further resuspended and homogenized in 0.5% sarkosyl extraction buffer and incubated overnight at 4° C. for extracting the insoluble rhMD protein. This was followed by centrifugation to generate a supernatant post-solubilization and an insoluble pellet. The supernatant post-solubilization was further filtered and loaded onto a HisTrap column using step elution at pH8. The loaded HisTrap chromatography column was washed and eluted to obtain elution fractions that were analyzed for detection of the purified rhMD protein. The presence of the four expressed rhMD proteins was detected in the respective post-lysis pellet (P1) and supernatant (S1), supernatant post-solubilization (S2) and insoluble pellet (P2), and the post-affinity purification eluants were determined by SDS-PAGE. The elution profile of the rhMD protein purified from the HisTrap chromatography column was also tracked.

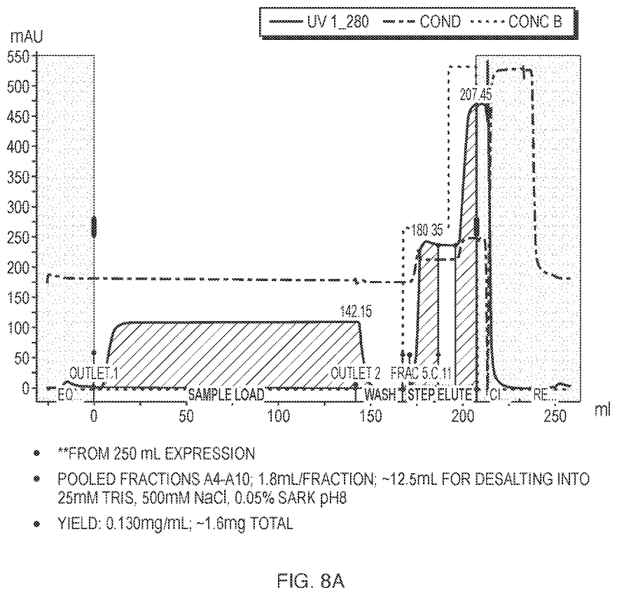

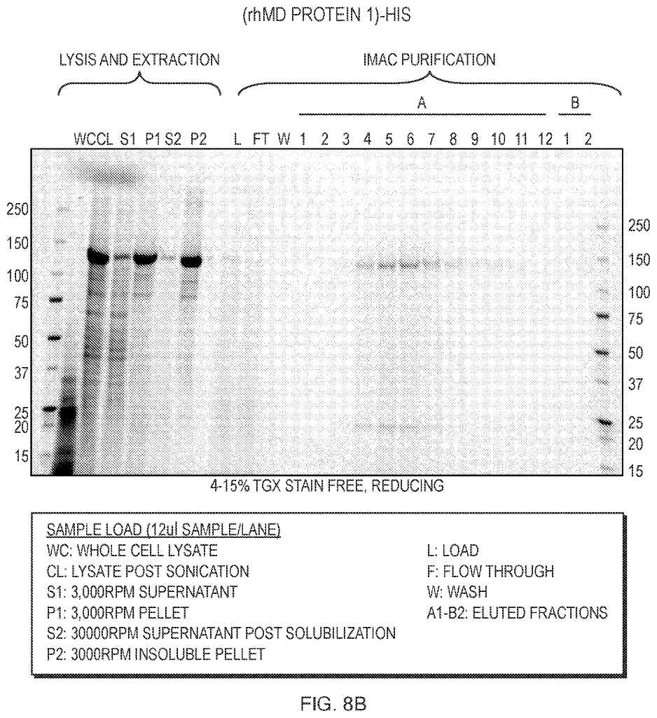

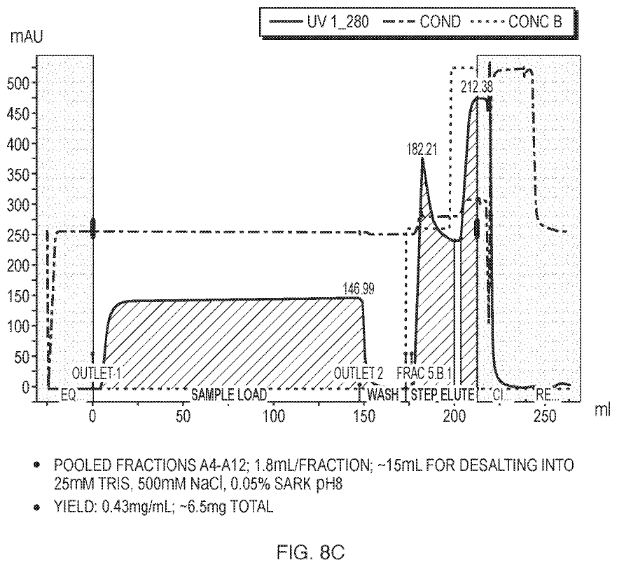

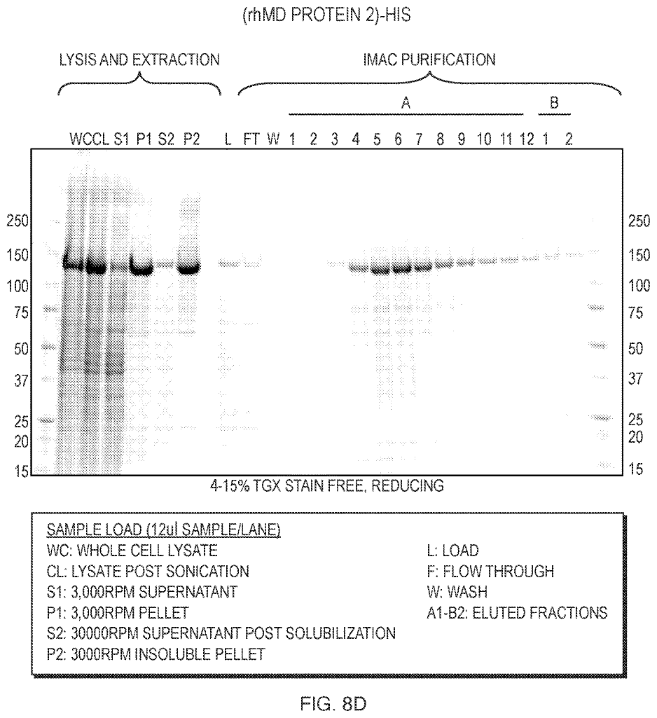

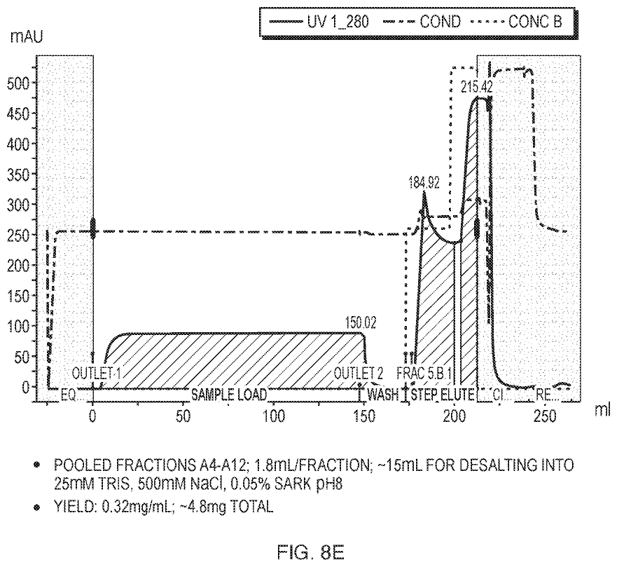

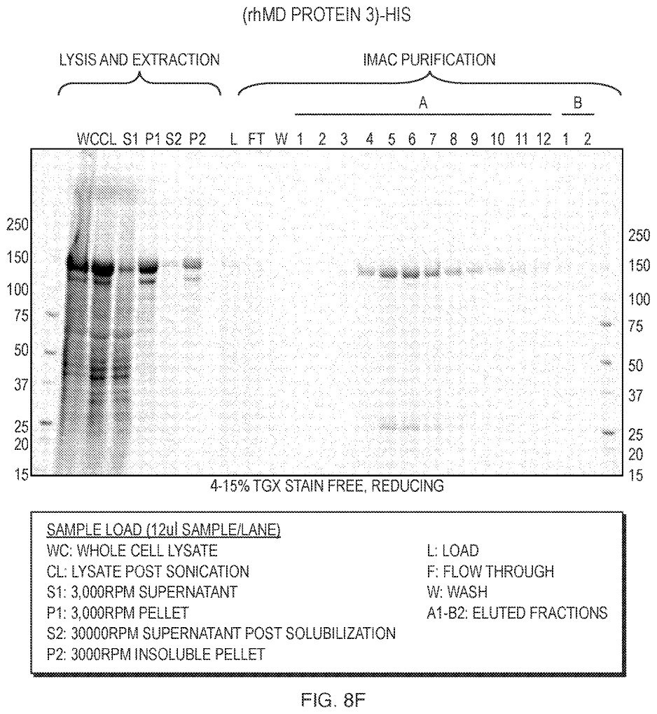

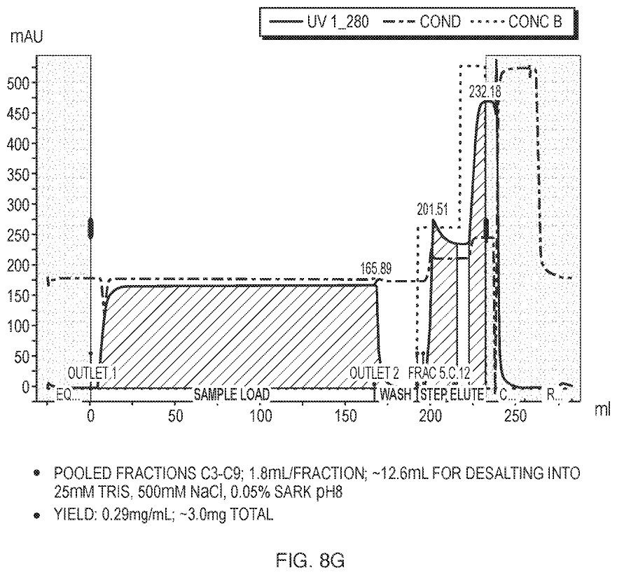

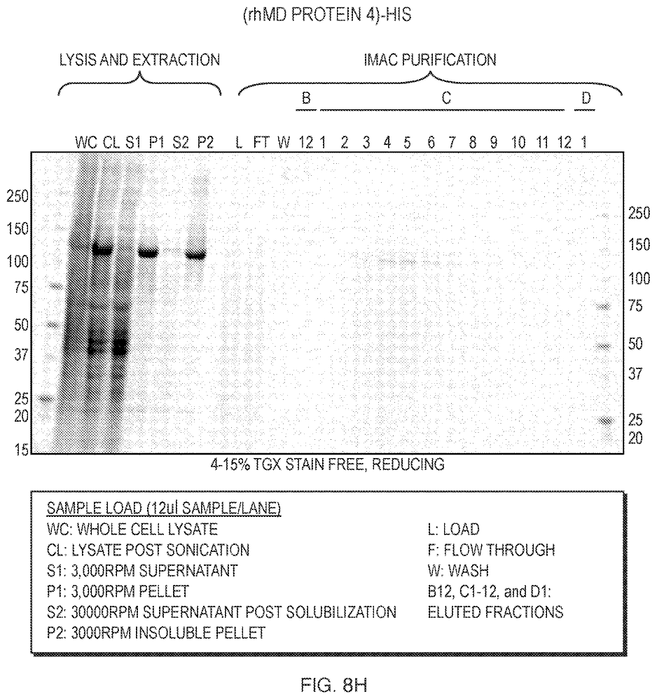

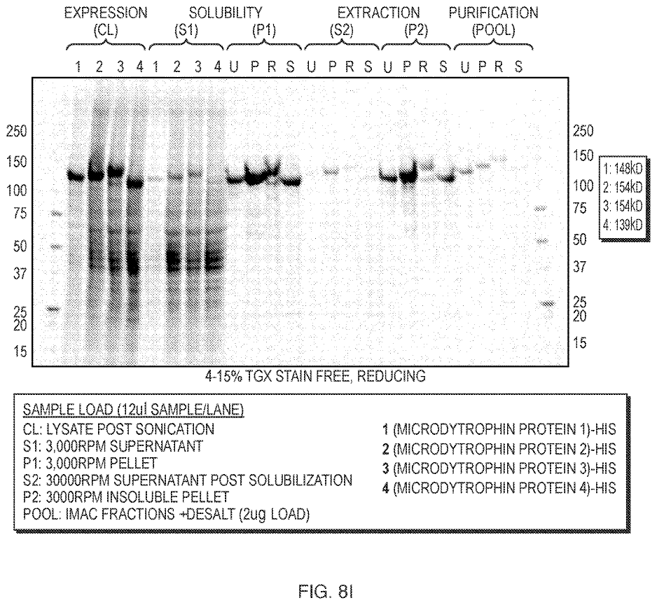

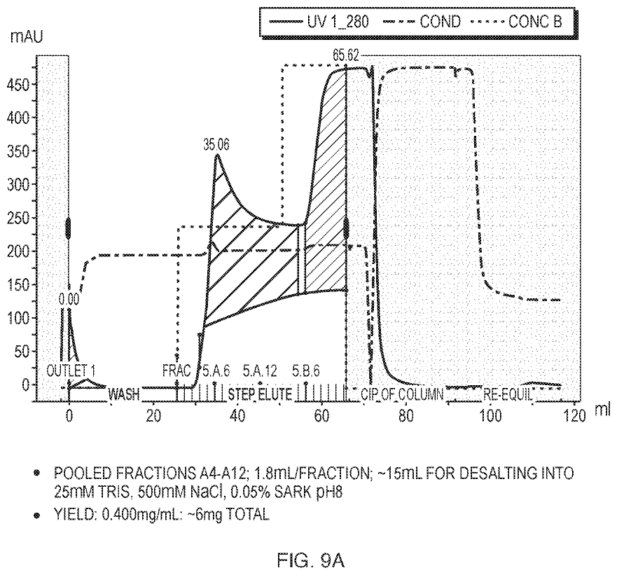

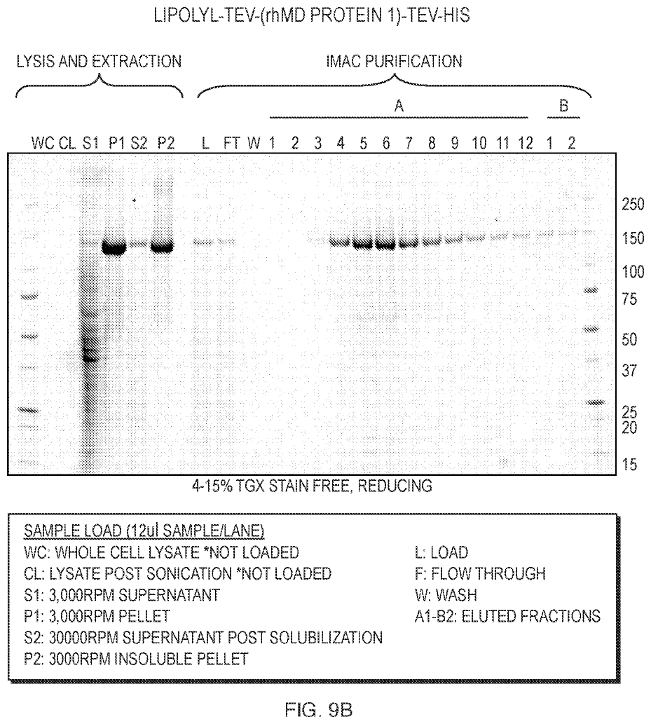

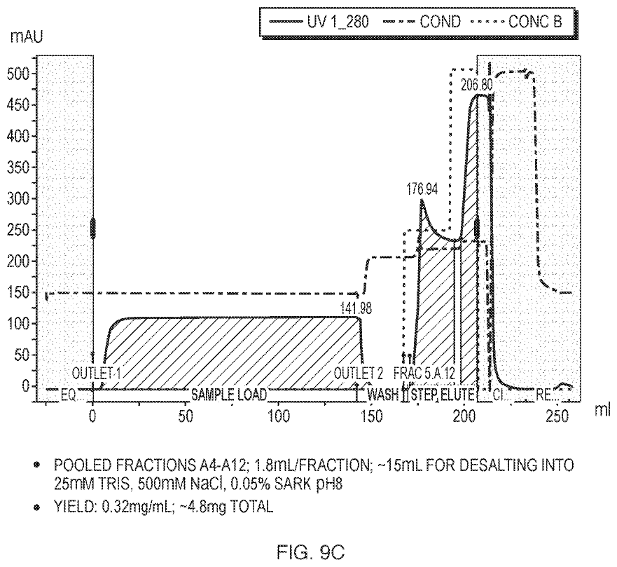

The results showed presence of significant amount of all four rhMD proteins in the supernatant obtained post-solubilization with 0.5% sarkosyl (S2) and in the eluent fractions (fractions A1-A12 and B1-B2 for FIGS. 8B, 8D, and 8F and fractions B12, C1-C12 and D1 for FIG. 8H) obtained from loading the S2 through the His Trap chromatography column (see FIGS. 8B, 8D, 8F, 8H, and 8I). The results indicated that the disclosed method of incubation of the post-lysis pellet of rhMD protein expressing cells in the 0.5% sarkosyl extraction buffer significantly improved solubility and yield of all rhMD proteins.

### Example 6: Effect of Lipolyl Solubility Tag and Sarkosyl Extraction on Solubility and Yield of Different rhMD Polypeptides

The effect of linking a solubility tag on the efficacy of the disclosed method of production of rhMD was further evaluated for improving the solubility of four different rhMD proteins linked with an N-terminal Lipoyl-TEV tag (solubility tag) and a C-terminal TEV-HIS tag (detection tag). (See FIG. 7A). Plasmid DNA constructs encoding four different rhMD proteins were separately transfected into Expi293 cells at 1.5 μg/ml and harvested 48 hours post transfection. The percentage viability, viable cell count, and cell pellet weight for the harvested cells for each construct are described in Table 2 below.

TABLE 2
Percentage viability, viable cell count, and cell pellet weight
for the harvested cells for rhMD protein constructs

| | | Viable Cell Count | Pellet wet |
|---|---|---|---|
| Construct | Viability (%) | (×10⁶ cells/ml) | weight (g) |
| Lipoyl-TEV-rhMD | 94.4 | 7.52 | 16.5 |
| protein 1-TEV-HIS | | | |
| Lipoyl-TEV-rhMD | 95 | 8.22 | 16.0 |
| protein 2-TEV-HIS | | | |
| Lipoyl-TEV-rhMD | 93 | 8.95 | 14.9 |
| protein 3-TEV-HIS | | | |
| Lipoyl-TEV-rhMD | 97 | 8.51 | 17.3 |
| protein 4-TEV-HIS | | | |

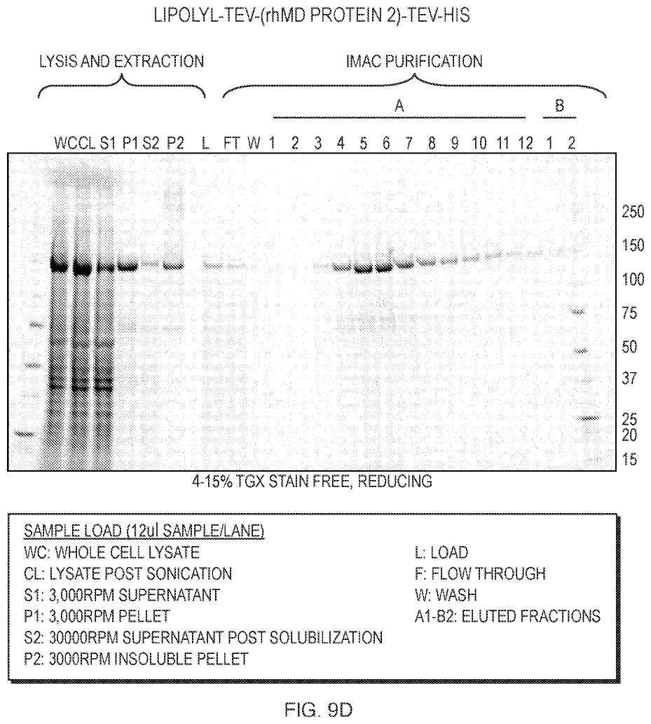

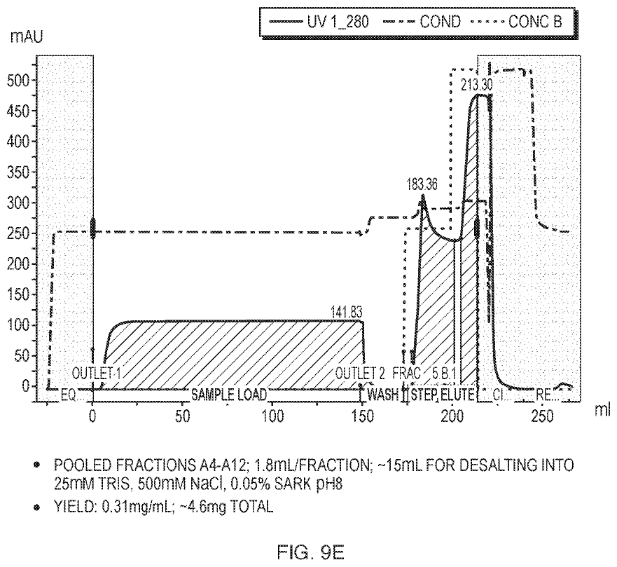

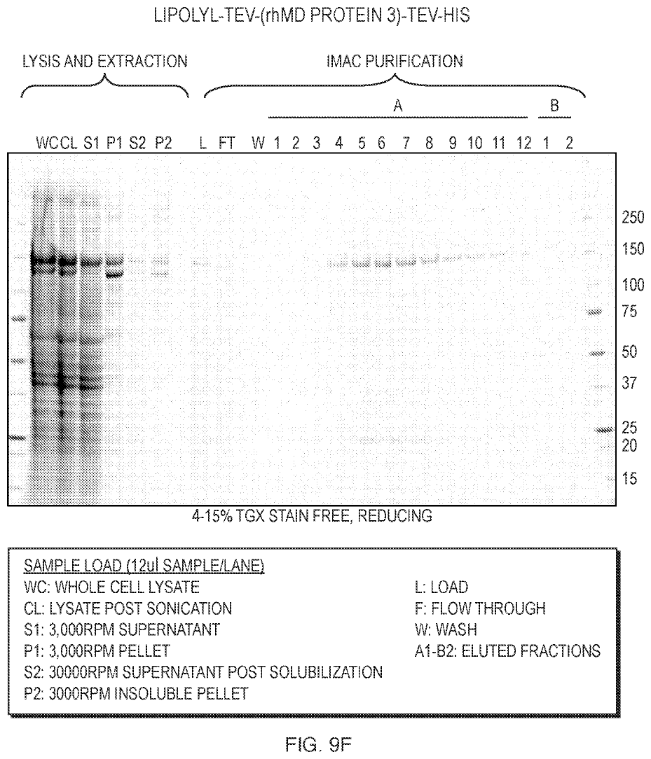

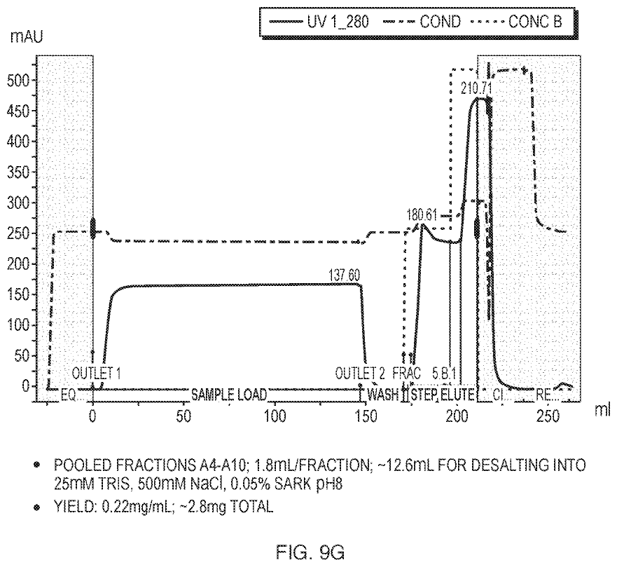

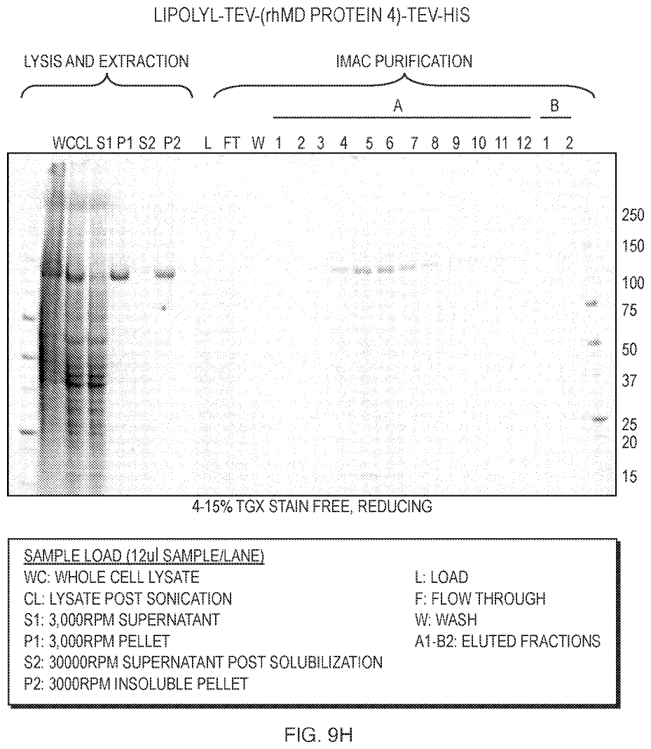

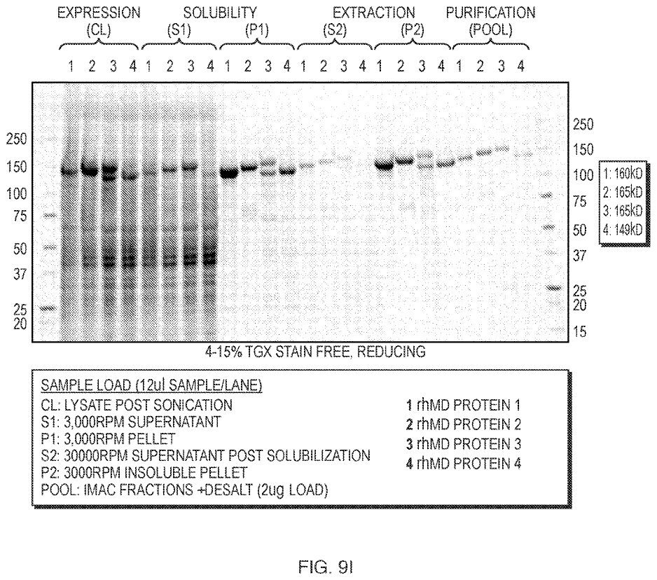

Details of the experimental procedure for isolating the expressed rhMD proteins are same as in Example 5 (also see, FIG. 7B). The results showed presence of significant amount of all four rhMD proteins in the supernatant obtained post-solubilization with 0.5% sarkosyl (S2) and in the eluent fractions (A1-A12 and B1-B2) obtained from loading the S2 through the HisTrap chromatography column (see FIGS. 9B, 9D, 9F, 9H, and 9I). The results indicated that linking the rhMD proteins to a Lipoyl solubility tag followed by extraction using the disclosed method of incubation of the post-lysis pellet of rhMD protein expressing cells in the 0.5% sarkosyl extraction buffer further improved solubility and yield of all rhMD proteins. Therefore, the method of production of this disclosure is applicable to increasing solubilization and production of different human microdystrophins.

### Example 7: Detection of Soluble rhMD by Anti-Dystrophin Antibodies

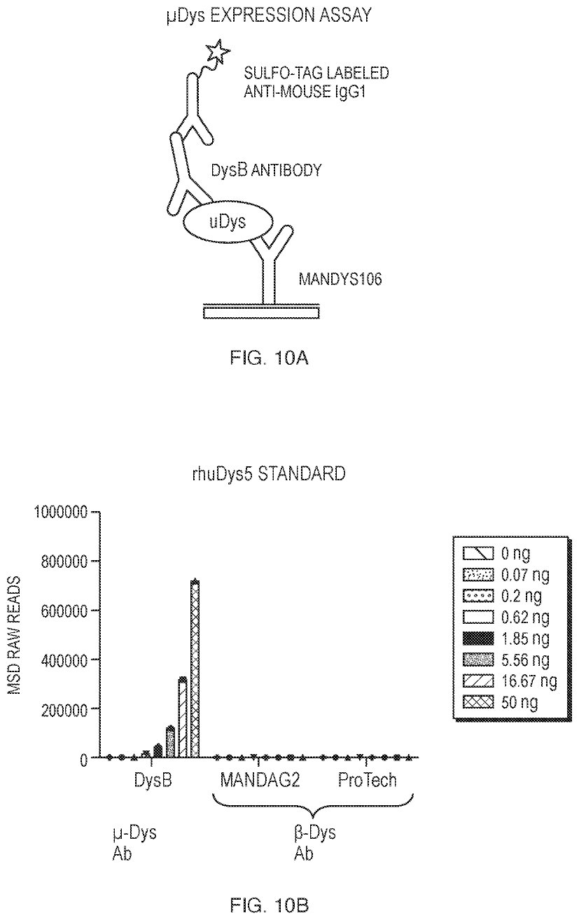

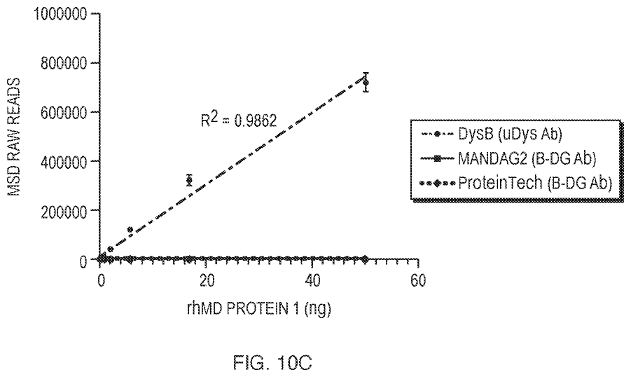

The study described herein determined the detection of rhMD in a standard solution comprising the rhMD produced by the disclosed method using commercially available Dystrophin antibodies in an enzyme linked immunosorbent assay (ELISA). (See FIG. 10A)

The results showed that rhMD protein was detected in dilutions of the rhMD standard solution using commercially available anti-microdystrophin specific antibody (DysB) with a linear relationship between the concentration of the rhMD and the ELISA detection signal. (See FIG. 10B-10C). Based on these results, the rhMD protein produced by the method of this disclosure is structurally stable and can be potentially used in in vitro analytical methods for detecting presence of microdystrophin peptides and microdystrophin-specific antibodies in samples.

### Example 8: Characterization of Soluble rhMD by Peptide Mapping

Peptide mapping by enzymatic digest and mass spectrometric analysis was carried out to characterize rHMD protein expressed and purified according to the foregoing examples. Briefly, rhMD protein 1 was expressed in Expi293 cells according to Example 1 above. The rHMD protein 1 (SEQ ID NO: 1) had a C-terminal HIS tag but no other solubility, detection, or affinity tags. The protein was purified using sarkosyl extraction according to Example 5 above.

A multi-digest approach using 6 highly specific digestion enzymes was used to achieve maximum coverage of the rhMD protein. Trypsin, AspN, Glu-C, Chymotrypsin Elastase, and Trypsin+Chymotrypsin proteases were used according to Table 3 to digest the purified rhMD protein.

TABLE 3
Multi-digest peptide mapping of rhMD protein

| | | | | Sequence |
|---|---|---|---|---|
| | | | Target | Coverage |
| Enzyme | Source | Digestion Conditions | Sequence | (%) |
| Trypsin | Promega | 2 μg protein digested for | C-terminus of R | 59 |
| | | 16 h at 37° C, in 50 mM | and K | |
| | | Ammonium Bicarbonate | | |
| | | at pH 8 with a 1:20 | | |
| | | enzyme:protein ratio | | |
| AspN | Promega | 2 μg protein digested for | N-terminus of D | 30 |
| | | 16 h at 37° C, in 50 mM | | |
| | | Tris/HCl at pH 8 with a | | |
| | | 1:20 enzyme:protein ratio | | |
| GluC | Thermo | 2 μg protein digested for | C-terminus of E | 12 |
| | Fisher | 16 h at 37° C, in 50 mM | | |
| | | Ammonium Bicarbonate | | |
| | | at pH 8 with a 1:20 | | |
| | | enzyme:protein ratio | | |
| Chymotrypsin | Promega | 2 μg protein digested for | C-terminus of | 64 |
| | | 16 h at 37° C, in 50 mM | W, F, L and Y | |
| | | Ammonium Bicarbonate | | |
| | | at pH 8 with a 1:20 | | |
| | | enzyme:protein ratio | | |
| Elastase | Thermo | 2 μg protein digested for | C-terminus of A, | 27 |
| | Fisher | 16 h at 37° C, in 50 mM | V, S, G, L and I | |
| | | Ammonium Bicarbonate | | |
| | | at pH 8 with a 1:20 | | |
| | | enzyme:protein ratio | | |
| Trypsin + | Promega | 2 μg protein digested for | C-terminus of R, | 64 |
| Chymotrypsin | | 16 h at 37° C, in 50 mM | K, W, F, L, and | |
| | | Ammonium Bicarbonate | Y | |
| | | at pH 8 with a 1:20 and | | |
| | | 1:50 enzyme:protein ratio, | | |
| | | respectively | | |

After digest, Sciex QTOF X500 was used for accurate mass measurement of unique protein fragments produced. The accurate mass measurement of the resulting peptides produced a set of m/z values that was compared against the in silico digestion of rhMD-HIS, comprising 1276 residues. In silico peptide identification was performed on LC-MS/MS data using Mascot (v 2.6.0.) and Protein Metrics (v5.2.31.). Mascot search and Protein Metrics search parameters were set to include up to two missed cleavages. Precursor mass tolerance was set to 10 ppm with fragment mass tolerance at 10 ppm.

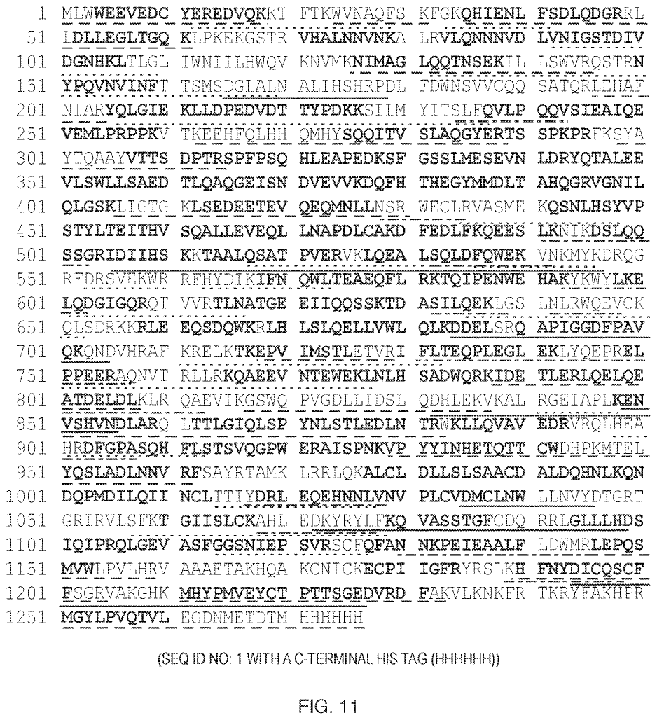

Mass analysis confirmed 90% sequence coverage of the rhMD protein. FIG. 11 shows the amino acid sequence of HIS-tagged rhMD protein 1 represented in 1-letter code with bolded type residues indicating confirmation of relative position and sequence and black residues indicating positions that could not be confirmed by peptide mapping (only 10% of the total sequence). Long dashed underlines represent individual digest fragments from trypsin proteolysis. Alternating long and short dash underlines represent individual digest fragments from elastase proteolysis. Dotted underlines represent individual digest fragments from chymotrypsin proteolysis. Solid underlines represent individual digest fragments from AspN proteolysis.

In addition, sample preparation relevant and potentially biologically relevant post-translational modifications were searched. Sample preparation-relevant post-translational modifications (i.e., those related to sample preparation) were observed as expected and included carbamidomethylation (C residues), deamidation (N and Q residues), and oxidation (M residues). Biologically relevant post-translational modifications not related to sample preparation were searched (Table 4). All modifications were considered variable, with the exception of carbamidomethylation of cysteines set as a fixed modification.

TABLE 4
Biologically relevant post-translational
modifications of rhMD protein

| Post-Translational | | Modified Positions |
|---|---|---|
| Modification | Affected Residues | Identified |
| Protein N-terminus | N-terminal residue | M1 |
| acetylation | | |
| Phosphorylation | S, T, Y | Not detected |
| O-glycosylation | S, T | Not detected |
| N-glycosylation | N | Not detected |

The MS/MS spectrum of any putative biologically relevant post-translational modification was manually interpreted. A high confidence spectrum assignment contains a high signal to noise, contiguous, series of amide backbone fragment ions inclusive of the potential modification.

These results confirm the identity and structural integrity of the rhMD protein expressed and purified by the methods described herein, demonstrating that these methods can be used with high confidence to prepare full length rhMD protein. These results further demonstrate the presence of N-terminus acetylation as a post-translational modification of rhMD protein. This PTM could not have been previously elucidated because reliable methods for preparing rhMD protein were lacking prior to the inventors' present disclosure.

### Example 9: Characterization of Soluble rhMD by SEC HPLC

Size exclusion chromatography (SEC) was performed to evaluate protein size and aggregation characteristics of rhMD-HIS. Briefly, rhMD-HIS was loaded on SEC column Agilent Bio SEC-5 (500 Å pore size, 4.6 mm diameter×300 mm length, 5 μm particle size; Agilent Bio part #PL5190-2533). High performance liquid chromatography (HPLC) conditions utilized a mobile phase buffer comprising 25 mM Tris, 500 mM NaCl pH8 with varying % sarkosyl and a 0.35 mL/min flow rate. Globular protein size standards were prepared using human IgM, thyroglobulin, and ferritin proteins. HPLC was performed at ambient room temperature using 10 μg to 30 μg of rhMD-HIS purified as described above (0.5% sarkosyl) and formulated in 25 mM Tris, 500 mM NaCl, 0.05% sarkosyl.

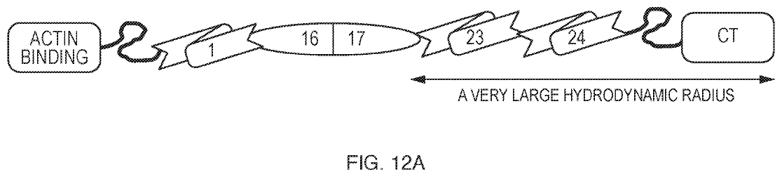

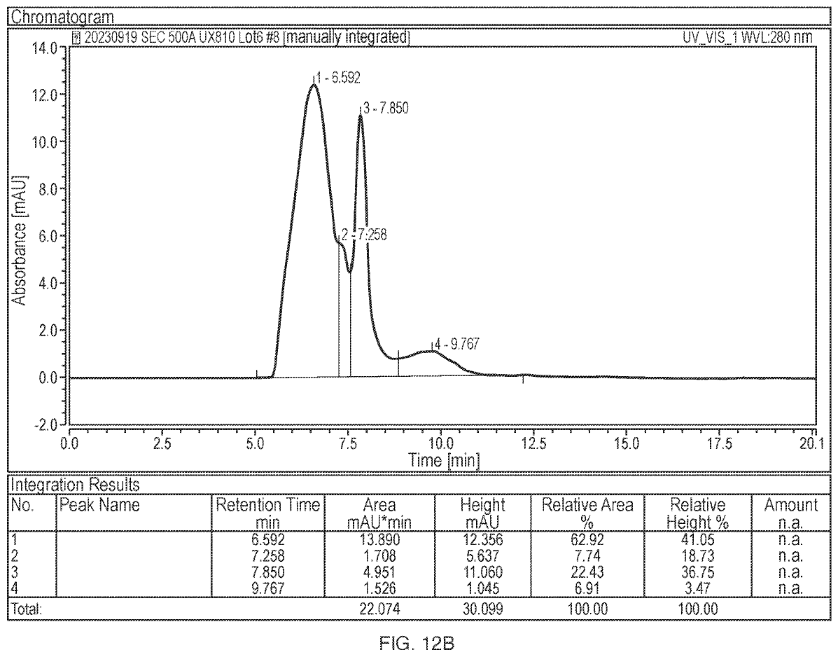

rhMD-HIS has a predicted molecular weight of 148.8 kDa, based on amino acid sequence. However, rhMD is predicted to have a rod-like structure with a very large hydrodynamic radius (FIG. 12A). This structure may lead the protein to perform in SEC analysis similar to a globular protein of significantly higher molecular weight. Indeed, using the globular protein molecular weight standards employed here, rhMD-HIS was found to elute in at least 2 peaks having predicted molecular weights corresponding to 622 kDa and 867 kDa (FIG. 12B). The 2 main peaks identified here suggest that rhMD-HIS may be present as a monomer (Peak 1) and dimer (Peak 3) as formulated.

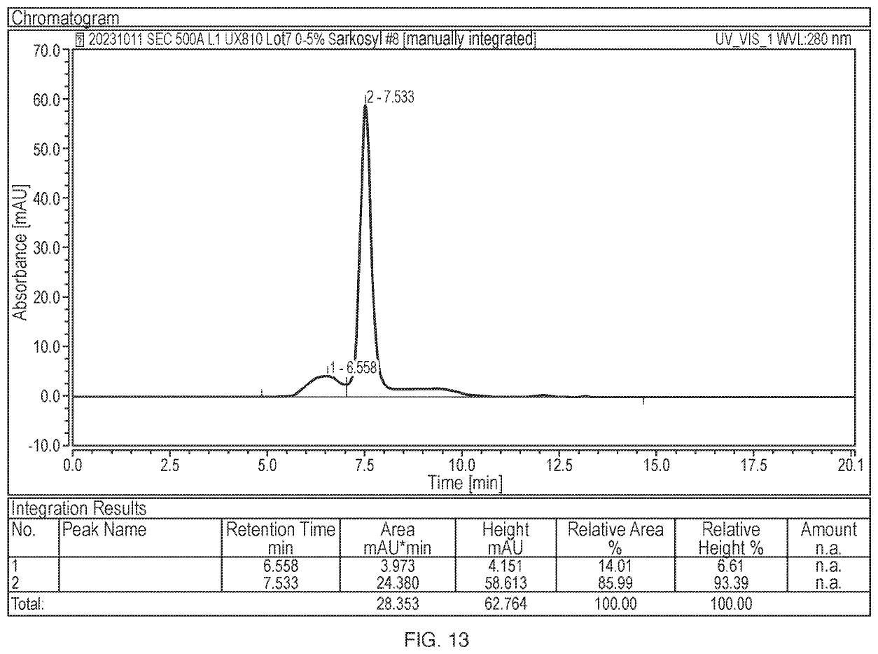

To determine if increased sarkosyl in the formulation buffer can improve stability of the rhMD monomer, the protein was next purified in 0.5% sarkosyl (as before) and formulated in the same buffer as before but with 0.5% sarkosyl (i.e., 0.5% sarkosyl maintained in purification and formulation conditions). SEC HPLC under these conditions resulted in a single main peak (FIG. 13), suggesting rhMD-HIS is present as a monomer when formulated in 0.5% sarkosyl.

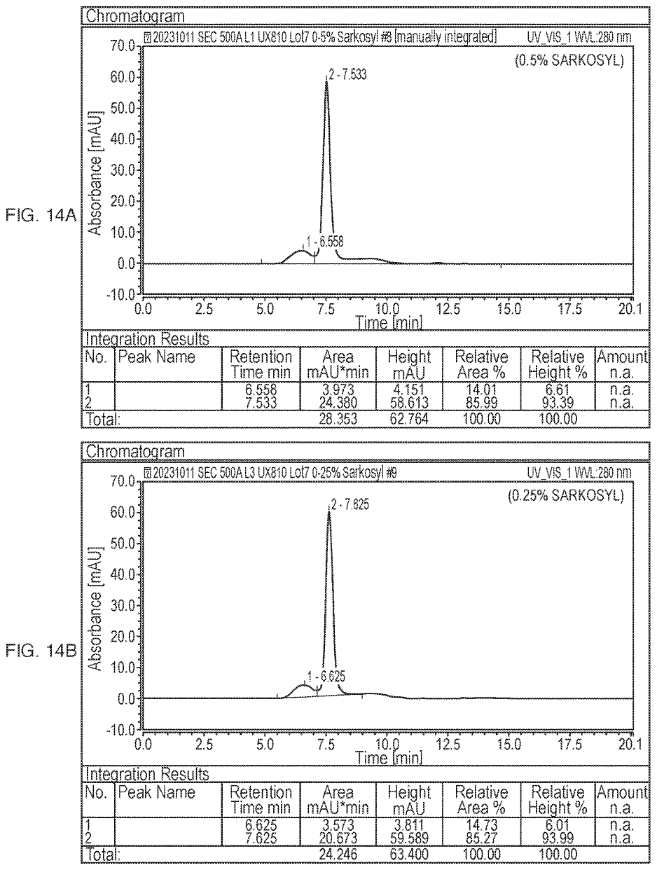

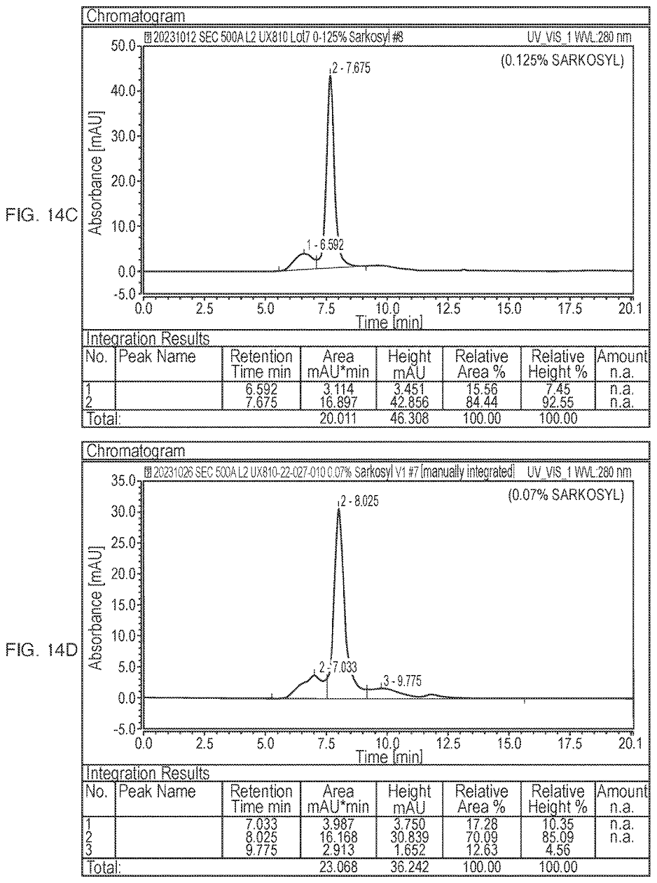

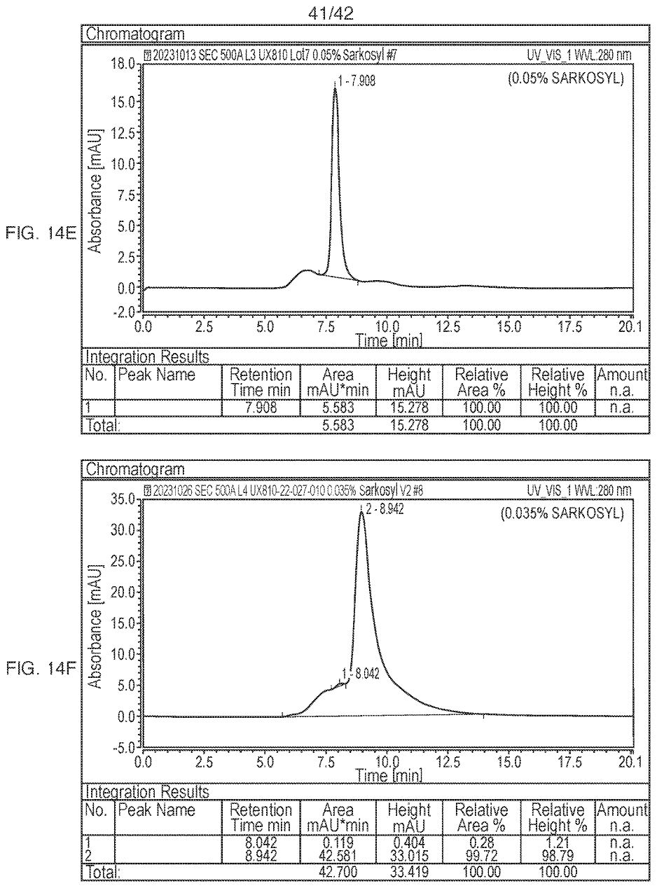

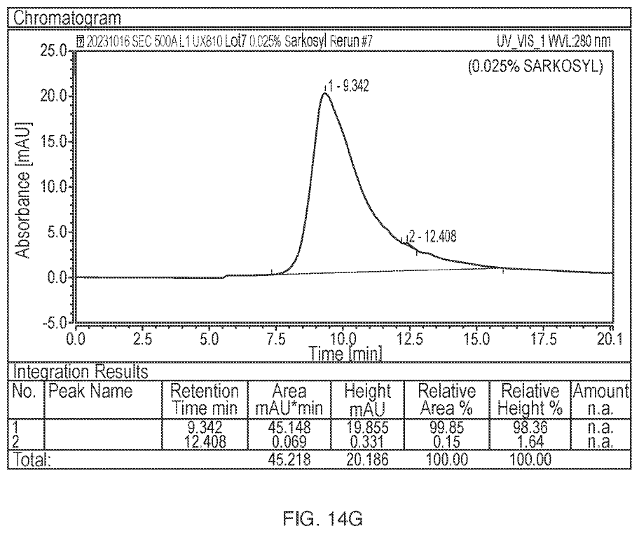

Further experiments were carried out to determine the minimal concentration of sarkosyl required for monomeric resolution by SEC HPLC. rhMD-HIS protein was purified in 0.5% sarkosyl and formulated in the same buffer as before but with 0.5%, 0.25%, 0.125%, 0.07%, 0.05%, 0.035%, or 0.025% sarkosyl. SEC HPLC resulted in a single main peak, suggesting monomeric form, at 0.5% down to 0.035% sarkosyl concentrations (FIG. 14A-F). At 0.25%, the rhMD-HIS peak broadens and skews toward higher molecular weights (FIG. 14G), suggesting a structural change or aggregation taking place in the purified protein.

These results show that sarkosyl stabilizes rhMD protein in monomeric form and the stabilizing effect is concentration-dependent. These conclusions were further supported by native gel electrophoresis experiments, in which rhMD-HIS was purified and reformulated in the same buffer as before but with differing sarkosyl concentrations and then run on a non-denaturing gel. The results revealed a single strong lower molecular weight band visible in 0.5% down to 0.125% sarkosyl (data not shown). This band tapered with decreasing detergent and was not visible at sarkosyl concentrations lower than 0.035%. Meanwhile, a second weaker higher molecular weight band appeared and increased slightly with reducing sarkosyl concentrations (data not shown). The results support the stabilizing effect of sarkosyl on purified rhMD protein, and suggest a minimum concentration of 0.035% sarkosyl is required after purification to maintain the protein in monomeric form.

### Example 10: Full-Length rhMD Protein for Bridging Anti-Drug Antibody (ADA) Assays

A bridging anti-drug antibody (ADA) assay to determine level of anti-rhMD specific antibody raised in a host administered with a therapeutic microdystrophin gene product is needed. Such a bridging ADA assay requires labelling the rhMD protein. Such a bridging ADA assay requires a set of two rhMD proteins: i) a full-length rhMD that is biotinylated and ii) a rhMD version that is labelled with a detectable label. However, microdystrophins may be highly disordered proteins prone to aggregation and falling out of solution, such that exposure to conjugation reagents for random labeling may cause the protein to aggregate or otherwise be difficult to utilize in sensitive assays. Alternatively, to avoid labeling rhMD, another labeled protein can be fused to the rhMD. Described herein is use of SpyTag/SpyCatcher technology to irreversibly conjugate a labelled recombinant SpyCatcher protein to rhMD protein. The study described herein, uses a full-length rhMD that comprises a biotinylatable tag according to SEQ ID NO: 55, that can be biotinylated at a single lysine residue in the tag, in vivo (rhMD biotinylatable protein) and a rhMD version comprising a peptide tag comprising an amino acid sequence according to any one of SEQ ID NOs: 47-49, e.g., rhMD protein 1-HIS-SpyTag (SEQ ID NO: 53) or rhMD protein 1-SpyTag-HIS (SEQ ID NO: 54), that can irreversibly bind a second peptide binding partner comprising an amino acid sequence according to any of SEQ ID NOs: 50-52, carrying a detectable label, such as Ruthenium (Ru). The second peptide binding partner can be labelled before conjugation to the rhMD protein to form a Ru-labelled rhMD second peptide binding partner-rhMD first peptide binding tag complex, and can further comprise a His-Tag for purification using a Ni-NTA column. The tagless biotinylated rhMD biotinylatable protein and the Ru-labelled rhMD protein complex can be incubated with samples containing anti-rhMD antibody to form a tri-partite complex which can then be affixed to streptavidin coated plates and whereby the level of anti-rhMD antibody in the sample can be determined by reading the Ru signal.

This approach could make the bridging assay more sensitive, removing the possibility of epitopes being blinded by the addition of a conjugated label.

### Example 11: Quantification of rhMD Protein in Mouse Tissue by Gel-LC/MS Assay

This example demonstrates an assay designed to detect small amounts of recombinant human microdystrophin (rhMD) protein, which remains a major analytical challenge in the field of developing rhMD-based gene therapeutics.

Approximately 20 mg of mouse muscle tissue from a mouse treated with a rhMD gene therapy adeno-associated virus vector was lysed with 8M urea, 2M thiourea, 50 mM Tris pH.8.0, 3% SDS, 10 mM TCEP lysis buffer. Mouse muscle proteins and a fixed amount of calibrator rhMD proteins (carbon-13 labeled rhMD protein 1 and nitrogen-15 labeled rhMD protein 1) prepared according to the methods provided herein were fractionated on an SDS-PAGE gel. For each sample, a 4 mm gel band was excised at 150 kDa. The excised band was digested with trypsin and the release of two peptides (one unique for digested rhMD protein 1, and the other shared between rhMD protein 1 and wild type dystrophin) and their corresponding ¹³C and ¹⁵N calibrators was used for quantitation.

The shared peptide common to digested dystrophin and rhMD protein 1 served as a qualifier and was found to have a quantitative range of 8 pg/mg to 125 ng/mg tissue. The peptide unique to digested rhMD protein 1 was found to have a quantitative range of 5.0-125 ng/mg tissue.

To evaluate the imprecisions of the assay, three muscle lysates displaying low, mid and high levels of dystrophin by western blot analysis were spiked with the two protein calibrators, ¹³C rhMD protein 1 and 15N rhMD protein 1, at 20 ng and 125 ng respectively and were analyzed. 3 replicates of 3 sets of samples were prepared per day for 3 non-consecutive days and analyzed with the micro-LC/7500 triple quadrupole system. The imprecision of the assay was calculated. The percent of coefficient of variation (% CV) for the intra-assay varied from 1% to 5% and from 3% to 16% for the inter-assay.

The Gel-LC/MS assay was demonstrated to monitor the release of a peptide unique to a rhMD protein and an additional peptide shared between a rhMD protein and dystrophin with good reproducibility. The assay is sensitive as it allows to work with a few milligrams of sample tissue.

### NUMBERED EMBODIMENTS

Embodiments disclosed herein include embodiments P1 to P100 as provided in the numbered embodiments of the disclosure.

Embodiment P1: A method of producing a recombinant human microdystrophin (rhMD) protein in solution, the rhMD protein comprising: an N-terminal actin binding domain (NTD); one to five Spectrin-Like Repeat (R) domains selected from R1 domain, R2 domain, R3 domain, R16 domain, R17 domain, R22 domain, R23 domain, and R24 domain; and a beta-dystroglycan binding domain; the method comprising: transfecting a host cell culture with an amount of a nucleic acid encoding the rhMD protein; maintaining the host cell culture for an amount of time for expression of the rhMD protein, wherein the expressed rhMD protein is associated with the cell membrane of the host cells; contacting the host cell culture with a lysis buffer for an amount of time to generate a cell lysate; centrifuging the cell lysate to generate a cell membrane fraction, said cell membrane fraction comprising plasma membranes of the cell culture; and contacting the cell membrane fraction with a membrane extraction buffer comprising an amount of an anionic detergent to release the rhMD protein from the cell membrane fraction.

Embodiment P2: A method of producing a recombinant human microdystrophin (rhMD) protein in solution from a host cell culture expressing the same, wherein the method comprises: contacting the host cell culture with a lysis buffer to generate a cell lysate; centrifuging the cell lysate to generate a cell membrane fraction, said cell membrane fraction comprising plasma membranes of the cell culture; contacting the cell membrane fraction with a membrane extraction buffer comprising an amount of an anionic detergent to release the rhMD protein from the cell membrane fraction; and purifying the released rhMD protein, wherein the rhMD comprises an N-terminal actin binding domain (NTD); one to five spectrin-like repeat (R) domains selected from R1 domain, R2 domain, R3 domain, R16 domain, R17 domain, R22 domain, R23 domain, and R24 domain; and a beta-dystroglycan binding domain.

Embodiment P3: The method of embodiment P1 or P2, wherein the rhMD protein comprises five R domains selected from R1 domain, R16 domain, R17 domain, R23 domain, and R24 domain, and a beta-dystroglycan binding domain.

Embodiment P4: The method of any one of embodiments P1-P3, wherein the rhMD protein further comprises at least a portion of a hinge domain selected from H1 domain, H2 domain, H3 domain, H4 domain and a Hinge like domain.

Embodiment P5: The method of embodiment P4, wherein the rhMD protein comprises at least a portion of 2 to 3 hinge domains selected from H1 domain, H2 domain, H3 domain, H4 domain and a Hinge like domain.

Embodiment P6: The method of embodiment P5, wherein the rhMD protein comprises a H1 domain and a H4 domain.

Embodiment P7: The method of embodiment P6, wherein the rhMD protein comprises an NTD coupled to a H1 domain coupled to a R1 domain coupled to a R16 domain coupled to a R17 domain coupled to a R23 domain coupled to a R24 domain coupled to a H4 domain coupled to a beta-dystroglycan binding domain.

Embodiment P8: The method of embodiment P7, wherein the rhMD protein comprises an amino acid sequence according to SEQ ID NO: 1 or having at least 90% sequence identity to the amino acid sequence set forth in SEQ ID NO: 1.

Embodiment P9: The method of embodiment P1 or P2, wherein the rhMD protein comprises a sequence according to any one of SEQ ID NO: 2, SEQ ID NO: 3, and SEQ ID NO: 4, or having at least 90% sequence identity to any one of the amino acid sequences set forth in SEQ ID NO: 2, SEQ ID NO: 3, and SEQ ID NO: 4.

Embodiment P10: The method of any one of embodiments P1-P9, wherein the rhMD protein further comprises a solubility tag selected from any one of: small ubiquitin-like modifier tag (SUMO tag); a Lipoyl domain tag (Lipoyl tag); and a combination thereof, wherein the solubility tag is located at the rhMD protein's N-terminus, at the rhMD protein's C-terminus, or both.

Embodiment P11: The method of embodiment P10, wherein the rhMD protein comprises at the rhMD protein's N-terminus any one of: a SUMO tag; a Lipoyl tag; and a combination thereof.

Embodiment P12: The method of embodiment P10, wherein the rhMD protein further comprises at the rhMD protein's C-terminus any one of: a SUMO tag; a Lipoyl tag; and a combination thereof.

Embodiment P13: The method of any one of embodiments P10 to P12, wherein the rhMD protein further comprises a detection tag at the rhMD protein's N-terminus, the rhMD protein's C-terminus, or both the rhMD protein's N- and C-termini, wherein said detection tag is any one of: a maltose binding protein tag (MBP tag); a glutathione S-transferase tag (GST tag); at least one histidine tag (HIS tag); a green fluorescence tag (GFP tag); at least one Strep tag sequence; and a combination thereof.

Embodiment P14: The method of embodiment P13, wherein the solubility tag, the detection tag, or both are linked to the rhMD protein by a Tobacco etch virus (TEV) protease cleavage site.

Embodiment P15: The method of embodiment P1 or P2, wherein, prior to contacting the host cell culture with the lysis buffer, the host cell culture is maintained for an amount of time such that the percentage of live cells (viability) of the host cell culture, before contacting the host cell culture with a lysis buffer is any one of: at least 50%; at least 70%; at least 90%; and at least 95%.

Embodiment P16: The method of embodiment P15, wherein the viability of the host cell culture is determined by a microscopy-based assay, colorimetry-based assay, flow-cytometry-based assay, or a combination thereof.

Embodiment P17: The method of embodiment P1 or P2, wherein, prior to contacting the host cell culture with the lysis buffer, the host cell culture is maintained for an amount of time such that the percentage of transfected cells in the host cell culture, before contacting the host cell culture with a lysis buffer is any one of: at least 50%; at least 70%; at least 90%; and at least 95%.

Embodiment P18: The method of embodiment P17, wherein the percentage of transfected cells is determined by a fluorescence microscopy-based assay, immunostaining-based assay, flow-cytometry-based assay, or a combination thereof.

Embodiment P19: The method of any one of embodiments P1 or P3 to P18, wherein the step of maintaining comprises incubating the host cell culture for at least 24 hours; at least 48 hours; at least 72 hours; or at least 96 hours.

Embodiment P20: The method of any one of embodiments P1 to P19, wherein the lysis buffer comprises a detergent selected from the group consisting of a non-ionic detergent, a cationic detergent, an anionic detergent, a zwitterionic detergent, and a combination thereof.

Embodiment P21: The method of embodiment P20, wherein the detergent is any one of Triton X-100, Triton X-114, Triton-200, sodium dodecyl sulfate, NP-40, Tween 20, Tween 80, and CHAPS.

Embodiment P22: The method of any one of embodiments P1 to P21, wherein the centrifuging of the cell lysate is done at 1100×g to 12000×g for about 5 min to about 1 hour.

Embodiment P23: The method of any one of embodiments P1 to P22, wherein the membrane extraction buffer comprises about 0.2% w/v to about 1.0% w/v of the anionic detergent.

Embodiment P24: The method of embodiment P23, wherein the membrane extraction buffer comprises about 0.3% w/v to about 0.5% w/v of the anionic detergent.

Embodiment P25: The method of embodiment P24, wherein the membrane extraction buffer comprises about 0.3% w/v of the anionic detergent.

Embodiment P26: The method of embodiment P25, wherein the anionic detergent is any one of sodium dodecyl sulfate (SDS), sodium deoxycholate (SD), sulfonic acid salt, alcohol sulfate, alkylbenzene sulfonate, phosphoric acid ester, and carboxylic acid salt.

Embodiment P27: The method of embodiment P25, wherein the anionic detergent is any one of ammonium lauryl sulfate, sodium laureth sulfate, sodium lauryl sarcosinate (sarkosyl), potassium lauryl sarcosinate sodium myreth sulfate, sodium pareth sulfate, sodium stearate, sodium lauryl sulfate, a olefin sulfonate, and ammonium laureth sulfate.

Embodiment P28: The method of embodiment P27, wherein the anionic detergent is sodium lauryl sarcosinate (sarkosyl).

Embodiment P29: The method of any one of embodiments P1 to P28, wherein contacting the cell membrane fraction with the membrane extraction buffer is carried out for about 16 minutes to about 24 minutes.

Embodiment P30: The method of any one of embodiments P1 to P29, wherein the method further comprises purifying the released rhMD protein using an affinity chromatography resin.

Embodiment P31: The method of embodiment P30, wherein the affinity chromatography resin is any one of a reduced-glutathione chromatography resin, a Ni-NTA chromatography resin, and an amylose chromatography resin.

Embodiment P32: Use of the recombinant human microdystrophin (rhMD) protein produced by the method of any one of embodiments P1 to P31 or a fragment thereof, in an analytical method for detecting a microdystrophin-specific antibody in a sample.

Embodiment P33: The use of embodiment P32, wherein the antibody is an anti-drug-antibody (ADA) raised in a subject administered a therapeutic microdystrophin gene or protein.

Embodiment P34: The use of any one of embodiments P32 to P33, wherein the analytical method is any one of a colorimetric method, enzyme immunoassay (EIA), radioimmunoassay (RIA), fluoroimmunoassay (FIA), chemiluminescent immunoassay (CLIA), and counting immunoassay (CIA).

Embodiment P35: The use of any one of embodiments P32 to P34, wherein the analytical method comprises: immobilizing the rhMD protein or a fragment thereof to a solid surface; contacting the immobilized rhMD protein with the sample for an amount of time sufficient for binding of the immobilized rhMD protein to the microdystrophin-specific antibody, if present in the sample, to form a bipartite complex of the rhMD protein and the microdystrophin-specific antibody; contacting the bipartite complex with a detection reagent that binds specifically to the microdystrophin-specific antibody and not the rhMD protein, for an amount of time to form a tripartite complex of the detection reagent, the rhMD protein, and the dystrophin-specific antibody, wherein the detection reagent comprises a detectable moiety that generates a detectable signal; and detecting the signal generated in step (c), wherein presence of the detectable signal indicates presence of microdystrophin-specific antibody in the sample, and absence of the detectable signal indicates absence of microdystrophin-specific antibody in the sample.

Embodiment P36: The use of embodiment P35, wherein the rhMD protein or fragment thereof is immobilized to the solid surface by any one of: a) an anti-microdystrophin antibody bound to the solid surface; and b) covalent crosslinking to the solid surface.

Embodiment P37: The use of embodiment P36, wherein the solid surface is any one of polyethylene, polyacrylamide, polystyrene, agarose, glass, and silicone rubber.

Embodiment P38: The use of any one of embodiments P35 to P37, wherein the analytical method further comprises: contacting the immobilized rhMD protein of step (a) of embodiment P35 with a coating/blocking buffer before proceeding with step (b) of embodiment P35; contacting the bipartite complex of step (b) of embodiment P35 with a wash buffer before proceeding with step (c) of embodiment P35; contacting the tripartite complex of step (c) of embodiment P35 with a wash buffer before proceeding with step (d) of embodiment P35; contacting the tripartite complex of step (c) of embodiment P35 with a detection solution before proceeding with step (d) of embodiment P35; or a combination thereof.

Embodiment P39: The use of any one of embodiments P35 to P38, wherein the detectable moiety is any one of an enzyme, a fluorophore, and a radioisotope.

Embodiment P40: The use of any one of embodiment P35 to P38, wherein the detectable signal is any one of a colorimetric signal, a fluorescent signal, and radiation.

Embodiment P41: The use of embodiment P35, wherein the detectable moiety is an enzyme, and step (c) further comprises contacting the tripartite complex with a substrate for generating the detectable signal.

Embodiment P42: The use of embodiment P41, wherein the enzyme is any one of a horse radish peroxidase, an alkaline phosphatase, a galactosidase, an acetylcholinesterase, and a catalase.

Embodiment P43: The use of embodiment P39, wherein the fluorophore is any one of fluorescein isothiocyanate (FITC), phycoerythrin (PE) or a conjugate thereof, an alexa fluor dye, a rhodamine dye, a Cy-based dye, a G-dye, and Texas red.

Embodiment P44: The use of embodiment P39, wherein the radioisotope is any one of ⁹⁰Y, ¹¹¹In, ¹⁷⁷Lu, ^{99m}Tc ¹²³I, ¹²⁵I, and ¹³¹I.

Embodiment P45: The use of any one of embodiments P35 to P44, wherein the detection reagent is an antibody or a fragment thereof.

Embodiment P46: The use of embodiment P45, wherein the detection reagent is any one of MANDYS106, Dys B, DYS1-CE, DYS2-CE, and DYS3-CE antibody.

Embodiment P47: Use of the recombinant human microdystrophin (rhMD) protein produced by the method of any one of embodiments P1 to P31, or a fragment thereof, in an analytical method of quantitatively measuring an amount of microdystrophin present in a sample solution.

Embodiment P48: The use of embodiment P47, wherein the analytical method is any one of a colorimetric method, enzyme immunoassay (EIA), radioimmunoassay (RIA), fluoroimmunoassay (FIA), chemiluminescent immunoassay (CLIA), and counting immunoassay (CIA).

Embodiment P49: The use of any one of embodiments P47 to P48, wherein the analytical method comprises: contacting the sample solution with a microdystrophin capture reagent and a microdystrophin detection reagent, and detecting a sample signal from the microdystrophin detection reagent in the sample solution; measuring the sample signal; contacting one or more reference solutions, each comprising a known amount of the rhMD protein, with a microdystrophin capture reagent and a microdystrophin detection reagent, and detecting one or more comparator signals from the microdystrophin detection reagent in each of the one or more solutions; measuring the one or more comparator signals; and comparing the sample signal with the one or more comparator signals to quantitatively determine the concentration of microdystrophin in the sample solution.

Embodiment P50: The use of embodiment P49, wherein the one or more reference solutions are dilutions of a standard solution and comprise from about 0.01 ng/ml to about 50 ng/ml of rhMD protein, wherein the standard solution comprises a known concentration of the rhMD protein produced according to the method of any one of embodiments P1 to P31, or a fragment thereof.

Embodiment P51: The use of embodiment P49 or P50, wherein the microdystrophin capture reagent has binding specificity for a microdystrophin in the sample solution and for the rhMD protein or a fragment thereof in the standard solution.

Embodiment P52: The use of embodiment P51, wherein the microdystrophin capture reagent is an antibody or a fragment thereof.

Embodiment P53: The use of any one of embodiments P49 to P52, wherein the microdystrophin capture reagent is immobilized to a solid surface.

Embodiment P54: The use of any one of embodiments P49 to P53, wherein the concentration of the microdystrophin capture reagent is from about 0.5 mg/ml to about 5 mg/ml.

Embodiment P55: The use of embodiment P53, wherein the microdystrophin capture reagent is immobilized by protein A to a solid surface.

Embodiment P56: The use of any one of embodiments P50 to P55, wherein the microdystrophin detection reagent binds specifically to a microdystrophin in the sample solution and the rhMD protein or a fragment thereof in the standard solution but does not bind to the microdystrophin capture reagent.

Embodiment P57: The use of embodiment P56, wherein the microdystrophin detection reagent is an antibody or a fragment thereof.

Embodiment P58: The use of any one of embodiments P49 to P57, wherein the microdystrophin detection reagent is present at a concentration of from about 0.5 ng/ml to about 2 mg/ml during step (a).

Embodiment P59: The use of any one of embodiments P49 to P58, wherein the microdystrophin capture reagent is any one of MANDYS106, Dys B, DYS1-CE, DYS2-CE, and DYS3-CE antibody.

Embodiment P60: The use of any one of embodiments P49 to P59, wherein the microdystrophin detection reagent is any one of Dys B, MANDYS106, DYS1-CE, DYS2-CE, and DYS3-CE antibody, and wherein the microdystrophin capture reagent is different from the microdystrophin detection reagent.

Embodiment P61: The use of any one of embodiments P49 to P60, wherein detecting a signal from the microdystrophin detection reagent comprises contacting the microdystrophin detection reagent with a secondary detection reagent that specifically binds the microdystrophin detection reagent and not the microdystrophin capture reagent.

Embodiment P62: The use of embodiment P61, wherein the secondary detection reagent is an antibody or a fragment thereof.

Embodiment P63: The use of any one of embodiments P61 to P62, wherein the secondary detection reagent comprises a reporter moiety.

Embodiment P64: The use of embodiment P63, wherein the reporter moiety is any one of a horse radish peroxidase, an alkaline phosphatase, a galactosidase, an acetylcholinesterase, and a catalase.

Embodiment P65: The use of any one of embodiments P49 to P64, wherein the analytical method further comprises contacting the sample solution and the one or more reference solutions comprising known amounts of the rhMD protein with a washing solution before measuring the sample signal.

Embodiment P66: The use of any one of embodiments P32 to P65, wherein the sample is from a subject suffering from or suspected of having a dystrophinopathy, optionally wherein the subject has been administered or is to be administered a therapeutic microdystrophin gene or protein.

Embodiment P67: A kit for detecting microdystrophin specific antibody in a sample, comprising: a recombinant human microdystrophin (rhMD) protein produced by the method of any one of embodiments P1 to P31 or a fragment thereof; and a detection reagent that specifically binds to the microdystrophin specific antibody but not to the rhMD protein or fragment thereof, wherein the detection reagent comprises a detectable moiety.

Embodiment P68: The kit of embodiment P67, further comprising: a solid surface; a coating/blocking solution; a washing solution; a detection solution; or a combination thereof.

Embodiment P69: The kit of any one of embodiments P67 to P68, wherein the rhMD protein or fragment thereof is immobilized to the solid surface.

Embodiment P70: The kit of embodiment P69, further comprising an anti-microdystrophin antibody or a crosslinker for immobilizing the rhMD protein or fragment thereof to the solid surface.

Embodiment P71: The kit of embodiment P70, wherein the solid surface is coated with protein A agarose.

Embodiment P72: The kit of embodiment P71, wherein the solid surface is any one of polyethylene, polyacrylamide, polystyrene, agarose, glass, and silicone rubber.

Embodiment P73: The kit of any one of embodiments P67 to P72, wherein the detectable moiety is any one of an enzyme, a fluorophore, and a radioisotope.

Embodiment P74: The kit of any one of embodiments P67 to P72, wherein the detectable moiety generates a detectable signal which is any one of a colorimetric signal, a fluorescent signal, and radiation.

Embodiment P75: The kit of embodiment P73, wherein the detectable moiety is an enzyme, and the kit further comprises a substrate that generates a detectable signal upon reacting with the enzyme.

Embodiment P76: The kit of embodiment P75, wherein the enzyme is any one of a horse radish peroxidase, an alkaline phosphatase, a galactosidase, an acetylcholinesterase, and a catalase.

Embodiment P77: The kit of embodiment P73, wherein the fluorophore is any one of fluorescein isothiocyanate (FITC), phycoerythrin (PE) or a conjugate thereof, an alexa fluor dye, a rhodamine dye, a Cy-based dye, a G-dye, and Texas red.

Embodiment P78: The kit of embodiment P73, wherein the radioisotope is any one of ⁹⁰Y, ¹¹¹In, ¹⁷⁷Lu, ^{99m}Tc ¹²³I, ¹²⁵I, and ¹³¹I.

Embodiment P79: The kit of any one of embodiments P67 to P78, wherein the detection reagent is an antibody or a fragment thereof.

Embodiment P80: The kit of embodiment P79, wherein the detection reagent is any one of MANDYS106, Dys B, DYS1-CE, DYS2-CE, and DYS3-CE antibody.

Embodiment P81: A kit for quantitatively measuring an amount of microdystrophin present in a sample solution comprising: a microdystrophin capture reagent; a microdystrophin detection reagent; and a standard solution comprising a recombinant human microdystrophin (rhMD) protein produced by the method of any one of embodiments P1 to P31 or a fragment thereof, or a lyophilized reagent for preparing said standard solution.

Embodiment P82: The kit of embodiment P81, wherein the microdystrophin detection reagent binds specifically to microdystrophin in the sample and the rhMD protein or a fragment thereof in the standard solution.

Embodiment P83: The kit of embodiment P82, wherein the microdystrophin detection reagent is an antibody or a fragment thereof.

Embodiment P84: The kit of any one of embodiments P81 to P83, further comprising: a solid surface; a coating solution; a washing solution; a detection solution; or a combination thereof.

Embodiment P85: The kit of any one of embodiments P81 to P84, wherein the microdystrophin capture reagent binds specifically to microdystrophin in the sample and the rhMD protein or a fragment thereof in the standard solution.

Embodiment P86: The kit of embodiment P85, wherein the microdystrophin capture reagent is an antibody or a fragment thereof.

Embodiment P87: The kit of embodiment P86, wherein the microdystrophin capture reagent is immobilized to the solid surface.

Embodiment P88: The kit of embodiment P87, wherein the microdystrophin capture reagent is immobilized by protein A to the solid surface.

Embodiment P89: The kit of any one of embodiments P81 to P88, wherein the concentration of the microdystrophin capture reagent is from about 0.5 mg/ml to about 5 mg/ml.

Embodiment P90: The kit of any one of embodiments P81 to P89, wherein the microdystrophin detection reagent binds specifically to microdystrophin in the sample and the rhMD protein or a fragment thereof in the standard solution but does not bind to the microdystrophin capture reagent.

Embodiment P91: The kit of embodiment P90, wherein the microdystrophin detection reagent is an antibody or a fragment thereof.

Embodiment P92: The kit of embodiment P91, wherein the microdystrophin detection reagent is used at a dilution of about 0.5 mg/ml to about 2 mg/ml.

Embodiment P93: The kit of any one of embodiments P81 to P92, wherein the microdystrophin capture reagent is any one of a Dys B, MANDYS106, Dys B, DYS1-CE, DYS2-CE, and DYS3-CE antibody.

Embodiment P94: The kit of any one of embodiments P81 to P93, wherein the microdystrophin detection reagent is any one of a Dys B, MANDYS106, DYS1-CE, DYS2-CE, and DYS3-CE antibody, and wherein the microdystrophin detection reagent is different from the microdystrophin capture reagent.

Embodiment P95: The kit of any one of embodiments P81 to P94, further comprising a secondary detection reagent that specifically binds the microdystrophin detection reagent and not the microdystrophin capture reagent.

Embodiment P96: The kit of embodiment P95, wherein the secondary detection reagent is an antibody or a fragment thereof.

Embodiment P97: The kit of any one of embodiments P95 to P96, wherein the secondary detection reagent comprises a reporter moiety.

Embodiment P98: The kit of embodiment P97, wherein the reporter moiety is any one of a horse radish peroxidase, an alkaline phosphatase, a galactosidase, an acetylcholinesterase, and a catalase.

Embodiment P99: A recombinant human microdystrophin (rhMD) protein produced according to the method of any one of embodiments P1 to P31, wherein the rhMD protein comprises an N terminal actin binding domain (NTD); at least a portion of 2 or 3 Hinge (H) domains selected from H1 domain, H2 domain, H3 domain, H4 domain, and a Hinge-like domain; one to five Spectrin-Like Repeat (R) domains selected from R1 domain, R2 domain, R3 domain, R16 domain, R17 domain, R22 domain, R23 domain, and R24 domain; and a beta-dystroglycan binding domain in solution.

Embodiment P100: The rhMD protein of embodiment P99, wherein the rhMD protein consists essentially of an NTD coupled to a H1 domain coupled to a R1 domain coupled to a R16 domain coupled to a R17 domain coupled to a R23 domain coupled to a R24 domain coupled to a H4 domain coupled to a beta-dystroglycan binding domain.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

1. A method of producing a recombinant human microdystrophin (rhMD) protein in solution, the rhMD protein comprising: an N-terminal actin binding domain (NTD); one to five Spectrin-Like Repeat (R) domains selected from R1 domain, R2 domain, R3 domain, R16 domain, R17 domain, R22 domain, R23 domain, and R24 domain; and a beta-dystroglycan binding domain; the method comprising:
(a) transfecting a host cell culture with a nucleic acid encoding the rhMD protein;
(b) expressing the rhMD protein in the host cell culture;
(c) generating a cell lysate by incubating the host cell culture with a lysis buffer;
(d) centrifuging the cell lysate to generate a cell membrane fraction, said cell membrane fraction comprising plasma membranes of the cell culture; and
(e) contacting the cell membrane fraction with a membrane extraction buffer comprising an amount of an anionic detergent to release the rhMD protein from the cell membrane fraction.

2-9. (canceled)

10. The method of claim 1, wherein the rhMD protein further comprises one or more solubility tags selected from:
(a) small ubiquitin-like modifier tag (SUMO tag);
(b) a Lipoyl domain tag (Lipoyl tag); and
(c) a combination thereof,
wherein the one or more solubility tag is located at the N-terminus of the rhMD protein, at the C-terminus of the rhMD protein, or both the N- and C-termini of the rhMD protein.

11-12. (canceled)

13. The method of claim 10, wherein the rhMD protein further comprises a detection tag at the N-terminus of the rhMD protein, C-terminus of the rhMD protein, or both the N- and C-termini of the rhMD protein, wherein said detection tag is any one of:
(a) a maltose binding protein tag (MBP tag);
(b) a glutathione S-transferase tag (GST tag);
(c) at least one histidine tag (HIS tag);
(d) a green fluorescence tag (GFP tag);
(e) at least one Strep tag sequence; and
(f) a combination thereof.

14-31. (canceled)

32. The method of claim 1, wherein the method further comprises purifying the released rhMD protein using an affinity chromatography resin.

33. (canceled)

34. A method of detecting a microdystrophin-specific antibody in a sample, the method comprising contacting the sample with the recombinant human microdystrophin (rhMD) protein produced by the method of claim 1 or a fragment thereof, and detecting binding between the microdystrophin-specific antibody and the rhMD protein or fragment thereof.

35. The method of claim 34, wherein the microdystrophin-specific antibody is an anti-drug-antibody (ADA) raised in a subject administered a therapeutic microdystrophin gene or protein.

36. (canceled)

37. The method of claim 34, comprising:
(a) immobilizing the rhMD protein or fragment thereof to a solid surface;
(b) contacting the immobilized rhMD protein with the sample for an amount of time sufficient for binding of the immobilized rhMD protein to the microdystrophin-specific antibody, if present in the sample, to form a bipartite complex of the rhMD protein and the microdystrophin-specific antibody;
(c) contacting the bipartite complex with a detection reagent that binds specifically to the microdystrophin-specific antibody and not the rhMD protein, for an amount of time to form a tripartite complex of the detection reagent, the rhMD protein, and the dystrophin-specific antibody,
wherein the detection reagent comprises a detectable moiety that generates a detectable signal; and
(d) detecting the signal generated in step (c),
wherein presence of the detectable signal indicates presence of microdystrophin-specific antibody in the sample, and absence of the detectable signal indicates absence of microdystrophin-specific antibody in the sample.

38-39. (canceled)

40. The method of claim 37, wherein the method further comprises:
contacting the immobilized rhMD protein of step (a) with a coating/blocking buffer before proceeding with step (b);
ii) contacting the bipartite complex of step (b) with a wash buffer before proceeding with step (c);
iii) contacting the tripartite complex of step (c) with a wash buffer before proceeding with step (d);
iv) contacting the tripartite complex of step (c) with a detection solution before proceeding with step (d); or
v) a combination thereof.

41-48. (canceled)

49. A method of quantitatively measuring an amount of microdystrophin present in a sample solution, the method comprising contacting the sample with the recombinant human microdystrophin (rhMD) protein produced by the method of claim 1, or a fragment thereof, and quantitatively measuring the amount of microdystrophin present in the sample solution.

50. (canceled)

51. The method of claim 49, wherein the method comprises:
(a) contacting the sample solution with a microdystrophin capture reagent and a microdystrophin detection reagent, and detecting a sample signal from the microdystrophin detection reagent in the sample solution;
(b) measuring the sample signal;
(c) contacting one or more reference solutions, each comprising a known amount of the rhMD protein or fragment thereof, with a microdystrophin capture reagent and a microdystrophin detection reagent, and detecting one or more comparator signals from the microdystrophin detection reagent in each of the one or more reference solutions;
(d) measuring the one or more comparator signals; and
(e) comparing the sample signal with the one or more comparator signals to quantitatively determine the concentration of microdystrophin in the sample solution.

52. The method of claim 51, wherein the one or more reference solutions are dilutions of a standard solution and comprise from about 0.01 ng/ml to about 50 ng/ml of rhMD protein, wherein the standard solution comprises a known concentration of the rhMD protein or fragment thereof.

53. The method of claim 51, wherein the microdystrophin capture reagent has binding specificity for a microdystrophin in the sample solution and for the rhMD protein or fragment thereof in the standard solution.

54-57. (canceled)

58. The method of claim 52, wherein the microdystrophin detection reagent binds specifically to a microdystrophin in the sample solution and the rhMD protein or fragment thereof in the standard solution but does not bind to the microdystrophin capture reagent.

59-62. (canceled)

63. The method of claim 51, wherein detecting a signal from the microdystrophin detection reagent comprises contacting the microdystrophin detection reagent with a secondary detection reagent that specifically binds the microdystrophin detection reagent and not the microdystrophin capture reagent.

64.-67. (canceled)

68. The method of claim 34, wherein the sample is from a subject suffering from or suspected of having a dystrophinopathy, optionally wherein the subject has been administered or is to be administered a therapeutic microdystrophin gene or protein.

69. A kit for detecting microdystrophin specific antibody in a sample, comprising:
(a) a recombinant human microdystrophin (rhMD) protein produced by the method of claim 1 or a fragment thereof; and
(b) a detection reagent that specifically binds to the microdystrophin specific antibody but not to the rhMD protein or fragment thereof, wherein the detection reagent comprises a detectable moiety.

70-82. (canceled)

83. A kit for quantitatively measuring an amount of microdystrophin present in a sample solution comprising:
(a) a microdystrophin capture reagent;
(b) a microdystrophin detection reagent; and
(c) a standard solution comprising a recombinant human microdystrophin (rhMD) protein produced by the method of claim 1 or a fragment thereof, or a lyophilized reagent for preparing said standard solution.

84. The kit of claim 83, wherein the microdystrophin detection reagent and/or the microdystrophin capture reagent binds specifically to microdystrophin in the sample and the rhMD protein or fragment thereof in the standard solution.

85-91. (canceled)

92. The kit of claim 83, wherein the microdystrophin detection reagent binds specifically to microdystrophin in the sample and the rhMD protein or fragment thereof in the standard solution but does not bind to the microdystrophin capture reagent.

93-100. (canceled)

101. A recombinant human microdystrophin (rhMD) protein produced according to the method of claim 1, wherein the rhMD protein comprises an N terminal actin binding domain (NTD); at least a portion of 2 or 3 Hinge (H) domains selected from H1 domain, H2 domain, H3 domain, H4 domain, and a Hinge-like domain; one to five Spectrin-Like Repeat (R) domains selected from R1 domain, R2 domain, R3 domain, R16 domain, R17 domain, R22 domain, R23 domain, and R24 domain; and a beta-dystroglycan binding domain in solution.

102. (canceled)
